(19) United States
(12) Patent Application Publication
(10) Pub. No.: US 2023/0338483 A1
(43) Pub. Date: Oct. 26, 2023

(54) METHOD FOR THE TREATMENT OF A DISEASE USING PIGMENT EPITHELIUM-DERIVED FACTOR (PEDF)

(71) Applicant: Curebiotech GmbH, Heidelberg (DE)

(72) Inventor: Ulrich Schraermeyer, Hechingen (DE)

(21) Appl. No.: 18/042,892

(22) PCT Filed: Sep. 3, 2021
(86) PCT No.: PCT/EP2021/074398

§ 371 Date: Feb. 24, 2023

(30) Foreign Application Priority Data
Sep. 3, 2020 (EP) 20194463.4
Sep. 8, 2020 (EP) 20195140.7

(51) Int. Cl. A61K 38/57; A61P 27/02; A61K 38/18; A61K 39/395

(52) U.S. Cl. CPC A61K 38/57; A61P 27/02; A61K 38/1866; A61K 39/3955

(57) ABSTRACT

The present invention is related to a pigment epithelium-derived factor (PEDF) for use in a method for treatment and/or prevention of dry macular degeneration in a subject, preferably dry age-related macular degeneration, wherein the method comprises administering PEDF and VEGF to the subject.

The present invention is related to a pigment epithelium-derived factor (PEDF) for use in a method for treatment and/or prevention of a disease, and an mRNA coding for a pigment epithelium-derived factor (PEDF) for use in a method for treatment and/or prevention of a disease.

Age-related macular degeneration (AMD) is the most common reason for legal blindness in western countries. Atrophy of the submacular retinal pigment epithelium and the development of choroidal neovascularization (CNV) results secondarily in loss of central visual acuity. Early signs of AMD are deposits (drusen) between retinal pigment epithelium and Bruch's membrane. There are two forms of AMD—wet and dry.

During the disease of wet AMD there is sprouting of choroid vessels into the subretinal space of the macula. These new vessels often develop abnormal, become leaky and cause subretinal edemas. These edemas lead to loss of central vision and reading ability.

In patients with dry AMD, the primary effect is loss of the choriocapillaris (Biesemeier, Taubitz et al. 2014). Subsequently, the retinal pigment epithelium (RPE) and the photoreceptors degenerate which leads to geographic atrophy (GA). For dry AMD there is at present no treatment available to prevent loss of the choriocapillaris.

Patients with subfoveal CNV currently were treated with drugs reducing or blocking vascular endothelial growth factor (VEGF). Since 2004, anti-VEGF therapy has become the standard treatment for wet AMD and has revolutionized the management of this disease. Between 2004 and 2006, three anti-VEGF drugs were introduced to ophthalmology after receiving regulatory approval for the treatment of AMD (pegaptanib, ranibizumab) or being used off-label (bevacizumab) (Browning, Kaiser et al. 2012). They exhibit important differences in their sites of activity, formulation methods, binding affinities and biological activities (Julien, Biesemeier et al. 2014). Pegaptanib (Macugen) is an oligonucleotide aptamer that selectively binds to and neutralizes the main pathological isoform of VEGF (VEGF-A165) by attaching to its heparin-binding domain. Ranibizumab (Lucentis, Genentech/Novartis) is an affinity matured, humanized, monoclonal antibody fragment (Fab), whereas bevacizumab (Avastin, Genentech/Roche) is a full-length, humanized monoclonal antibody. Both work by blocking the receptor-binding domain of all isoforms of VEGF-A (Ferrara, Damico et al. 2006). Aflibercept (VEGF Trap-Eye, Eylea, Regeneron/Bayer) is an anti-VEGF agent recently approved by the Food and Drug Administration. It is a fully human, recombinant fusion protein composed of the second immunoglobulin (Ig)-binding domain of VEGFR1 and the third Ig-binding domain of VEGFR2 fused to the fragment crystallizable (Fc) region of human IgG1.

Aflibercept binds to all VEGF-A isoforms, VEGF-B and P1GF (Papadopoulos, Martin et al. 2012). The effects of intravitreally injected bevacizumab in the eyes of monkeys have been extensively described (Peters, Heiduschka et al. 2007, Julien, Biesemeier et al. 2013, Schraermeyer and Julien 2013). The effects included reductions in choriocapillaris fenestrations, photoreceptor damage, formation of immune complexes and thrombotic microangiopathy. A prevailing rationale for thrombosis after bevacizumab treatment was presented by Meyer and colleagues (Meyer, Robles-Carrillo et al. 2009). They found that bevacizumab can induce platelet aggregation, degranulation and thrombosis through complex formation with VEGF, heparin and activation of the platelet Fc gamma RIIa receptor. Moreover, other results have demonstrated effective binding of the Fc domain of bevacizumab to human RPE and human umbilical vascular endothelial cell membranes via Fc receptors or membrane-bound VEGF, activating the complement cascade and leading to cell death (Meyer and Holz 2011). It is unclear whether there is a similar problem with aflibercept as it also contains the Fc domain of human IgG1. Furthermore, the IgG1 isotype is known to be very effective in the activation of the complement system through the classical pathway (Daha, Banda et al. 2011). Indeed, the Fc portion of IgG1 has a high ability to bind C1q causing subsequent activation of the classical pathway (Daha, Banda et al. 2011). In contrast, ranibizumab does not possess the Fc domain avoiding activation of the complement cascade, but nevertheless also induces hemolysis and fibrin formation in non-clinical studies (Julien, Biesemeier et al. 2014).

It was reported that VEGF inhibition can activate thrombocytes in humans treated for cancer (Meyer, Robles-Carrillo et al. 2009) or for neovascular AMD (Schraermeyer and Julien 2013). In addition, VEGF drugs after intravitreal application induced thrombotic microangiopathy in the choriocapillaris of monkeys (Peters, Heiduschka et al. 2007, Schraermeyer and Julien 2012). Anti-VEGF drugs also induce hemolysis, stasis and fibrin formation within the choriocapillaris (Schraermeyer and Julien 2012, Schraermeyer and Julien 2013, Julien, Biesemeier et al. 2014). Avastin forms together with heparin and VEGF protein complexes that induce thrombotic events (Julien, Biesemeier et al. 2013). In blood vessels of surgically excised choroidal membranes from patients suffering from wet AMD, anti-VEGF (bevacizumab) treatment induced thrombosis and protein complex formation (Schraermeyer, Julien et al. 2015).

These side effects are of disadvantage because AMD patients, due to their age, have higher risks to suffer from stroke or other vessel related diseases. Thus, an increased long-term mortality in individuals with wet AMD treated with bevacizumab compared to a same age and gender group without wet AMD was reported (Hanhart, Comaneshter et al. 2017). Particularly after myocardial infarction (Hanhart, Comaneshter et al. 2018) and after a cerebrovascular event (Hanhart, Comaneshter et al. 2018), the mortality caused by anti-VEGF treatment is largely enhanced. Moreover, long term treatment with anti-VEGF drugs causes loss of the original choriocapillaris and geographic atrophy in the peripheral parts of the retina in patients with wet AMD (Schutze, Wedl et al. 2015). Thus, this treatment induces additional visual loss that would not have occurred without this treatment.

Recently, due to the new possibility of using angiography together with ocular coherence tomography (OCTA) (Treister, Nesper et al. 2018) overcoming the short-coming of earlier fluorescein angiography only detecting CNV after leakage had already occurred, there is detected a notable prevalence of subclinical CNV in fellow eyes with unilateral exudative CNV, and significantly greater choriocapillaris nonperfusion adjacent to all CNV lesions. Treister et al. (Treister et al. 2018) identified a trend for increased choriocapillaris nonperfusion in exudative AMD eyes as compared with their fellow subclinical CNV eyes. This clearly shows that subclinical CNVs exist without reducing the visual acuity of these patients. These new findings support an earlier observation in eyes with late wet AMD in which neovascularization could help photoreceptors to survive (Biesemeier, Julien et al. 2014).

Regarding the long history for treatment of wet AMD, always the same principle as been used, namely removing or blocking the newly formed blood vessels. In order to achieve this, different methods have been used: laser coagulation, surgery, radiation, photodynamic therapy and today intravitreal anti-VEGF drugs. Whereas none of the first methods could improve vision, usage of anti-VEGF (ranibizumab) was successful and can improve visual acuity for a while but is still far away from an optimal rescue.

The problem underlying the present invention is the provision of a means for the treatment of ocular diseases such as age-related macular degeneration (AMD).

A further problem underlying the present invention is the provision of a means for the treatment of ocular disease such as age-related macular degeneration (AMD) providing improved visual acuity for a prolonged period of time.

These and other problems underlying the present invention are solved by the subject matter of the attached independent claims. Preferred embodiments may be taken from the attached dependent claims.

The problem underlying the present invention is also solved in a first aspect, which is also a first embodiment of the first aspect, by a pigment epithelium-derived factor (PEDF) for use in a method for treatment and/or prevention of a disease, wherein the method comprises administering PEDF to a subject and wherein treatment and/or prevention of a disease comprises inhibiting labyrinth capillary formation, inducing growth of choriocapillaris, preferably inducing growth of non-leaking choriocapillaris, tightening choriocapillaris, inhibiting extracellular matrix formation, protecting choriocapillaris, and/or guiding vessel development.

In a second embodiment of the first aspect, which is also an embodiment of the first embodiment of the first aspect, the disease is an eye disease.

In a third embodiment of the first aspect, which is also an embodiment of the second embodiment of the first aspect, the eye disease is macular degeneration, preferably macular degeneration is age-related macular degeneration (AMD), more preferably dry age-related macular degeneration or wet age-related macular degeneration.

In a fourth embodiment of the first aspect, which is also an embodiment of the third embodiment of the first aspect, PEDF inhibits growth and/or formation of geographic atrophy in wet AMD and/or dry AMD.

In a fifth embodiment of the first aspect, which is also an embodiment of the second embodiment of the first aspect, the disease is selected from the group comprising central serous chorioretinopathy, diabetic retinopathy, rubeosis iridis, corneal neovascularization, polypoidal choroidal vasculopathy, retinopathy of the prematurity and retinal and choroidal fibrosis.

In a sixth embodiment of the first aspect, which is also an embodiment of the fifth embodiment of the first aspect, PEDF inhibits the progression of retinal and/or choroidal fibrosis.

The problem underlying the present invention is also solved in a second aspect, which is also a first embodiment of the second aspect, by an mRNA coding for a pigment epithelium-derived factor (PEDF) for use in a method for treatment and/or prevention of a disease, wherein the method comprises administering the mRNA coding for PEDF to a subject and wherein treatment and/or prevention of a disease comprises inhibiting labyrinth capillary formation, inducing growth of choriocapillaris, preferably inducing growth of non-leaking choriocapillaris, tightening choriocapillaris, inhibiting extracellular matrix formation, protecting choriocapillaris, and/or guiding vessel development.

In a second embodiment of the second aspect, which is also an embodiment of the first embodiment of the second aspect, the disease is an eye disease.

In a third embodiment of the second aspect, which is also an embodiment of the second embodiment of the second aspect, the eye disease is macular degeneration, preferably macular degeneration is age-related macular degeneration (AMD), more preferably dry age-related macular degeneration or wet age-related macular degeneration.

In a fourth embodiment of the second aspect, which is also an embodiment of the third embodiment of the second aspect, PEDF inhibits growth and/or formation of geographic atrophy in wet AMD and/or dry AMD.

In a fifth embodiment of the second aspect, which is also an embodiment of the second embodiment of the second aspect, the disease is selected from the group comprising central serous chorioretinopathy, diabetic retinopathy, rubeosis iridis, corneal neovascularization, polypoidal choroidal vasculopathy, retinopathy of the prematurity and retinal and choroidal fibrosis.

In a sixth embodiment of the second aspect, which is also an embodiment of the fifth embodiment of the second aspect, PEDF inhibits the progression of retinal and/or choroidal fibrosis.

The problem underlying the present invention is also solved in a third aspect, which is also a first embodiment of the third aspect, by a pigment epithelium-derived factor (PEDF) or an mRNA coding for a pigment epithelium-derived factor (PEDF) for use in a method for treatment and/or prevention of a disease, wherein the method comprises administering the PEDF or the mRNA coding for PEDF to a subject, wherein the disease is an eye disease.

In a second embodiment of the third aspect, which is also an embodiment of the first embodiment of the third aspect, treatment and/or prevention of the disease comprises inhibiting labyrinth capillary formation, inducing growth of choriocapillaris, preferably inducing growth of non-leaking choriocapillaris, tightening choriocapillaris, inhibiting extracellular matrix formation, protecting choriocapillaris, and/or guiding vessel development.

In a third embodiment of the third aspect, which is also an embodiment of the first and second embodiment of the third aspect, the eye disease is macular degeneration, preferably macular degeneration is age-related macular degeneration (AMD), more preferably dry age-related macular degeneration or wet age-related macular degeneration.

In a fourth embodiment of the third aspect, which is also an embodiment of the third embodiment of the third aspect, PEDF inhibits growth and/or formation of geographic atrophy in wet AMD and/or dry AMD.

In a fifth embodiment of the third aspect, which is also an embodiment of the second embodiment of the third aspect, the disease is selected from the group comprising central serous chorioretinopathy, diabetic retinopathy, rubeosis iridis, corneal neovascularization, polypoidal choroidal vasculopathy, retinopathy of the prematurity and retinal and/or choroidal fibrosis.

In a sixth embodiment of the third aspect, which is also an embodiment of the fifth embodiment of the third aspect, PEDF inhibits the progression of retinal and/or choroidal fibrosis.

The problem underlying the present invention is also solved in a fourth aspect, which is also a first embodiment of the fourth aspect, by a pigment epithelium-derived factor (PEDF) or an mRNA coding for a pigment epithelium-derived factor (PEDF) for use in a method for treatment and/or prevention of a disease, wherein the method comprises administering the PEDF or the mRNA coding for PEDF to a subject, wherein the disease is macular degeneration.

In a second embodiment of the fourth aspect, which is also an embodiment of the first embodiment of the fourth aspect, treatment and/or prevention of the disease comprises inhibiting labyrinth capillary formation, inducing growth of choriocapillaris, preferably inducing growth of non-leaking choriocapillaris, tightening choriocapillaris, inhibiting extracellular matrix formation, protecting choriocapillaris, and/or guiding vessel development.

In a third embodiment of the fourth aspect, which is also an embodiment of the first and second embodiment of the fourth aspect, the macular degeneration is age-related macular degeneration (AMD), more preferably dry age-related macular degeneration or wet age-related macular degeneration.

In a fourth embodiment of the fourth aspect, which is also an embodiment of the third embodiment of the fourth aspect, PEDF inhibits growth and/or formation of geographic atrophy in wet AMD and/or dry AMD.

The problem underlying the present invention is also solved in a fifth aspect, which is also a first embodiment of the fifth aspect, by a pigment epithelium-derived factor (PEDF) or an mRNA coding for a pigment epithelium-derived factor (PEDF) for use in a method for treatment and/or prevention of a disease, wherein the method comprises administering the PEDF or the mRNA coding for PEDF to a subject, wherein the disease is central serous chorioretinopathy.

In a second embodiment of the fifth aspect, which is also an embodiment of the first embodiment of the fifth aspect, treatment and/or prevention of the disease comprises inhibiting labyrinth capillary formation, inducing growth of choriocapillaris, preferably inducing growth of non-leaking choriocapillaris, tightening choriocapillaris, inhibiting extracellular matrix formation, protecting choriocapillaris, and/or guiding vessel development.

The problem underlying the present invention is also solved in a sixth aspect, which is also a first embodiment of the sixth aspect, by a pigment epithelium-derived factor (PEDF) or an mRNA coding for a pigment epithelium-derived factor (PEDF) for use in a method for treatment and/or prevention of a disease, wherein the method comprises administering the PEDF or the mRNA coding for PEDF to a subject, wherein the disease is diabetic retinopathy.

In a second embodiment of the sixth aspect, which is also an embodiment of the first embodiment of the sixth aspect, treatment and/or prevention of the disease comprises inhibiting labyrinth capillary formation, inducing growth of choriocapillaris, preferably inducing growth of non-leaking choriocapillaris, tightening choriocapillaris, inhibiting extracellular matrix formation, protecting choriocapillaris, and/or guiding vessel development.

The problem underlying the present invention is also solved in a seventh aspect, which is also a first embodiment of the seventh aspect, by a pigment epithelium-derived factor (PEDF) or an mRNA coding for a pigment epithelium-derived factor (PEDF) for use in a method for treatment and/or prevention of a disease, wherein the method comprises administering the PEDF or the mRNA coding for PEDF to a subject, wherein the disease is rubeosis iridis.

In a second embodiment of the seventh aspect, which is also an embodiment of the first embodiment of the seventh aspect, treatment and/or prevention of the disease comprises inhibiting labyrinth capillary formation, inducing growth of choriocapillaris, preferably inducing growth of non-leaking choriocapillaris, tightening choriocapillaris, inhibiting extracellular matrix formation, protecting choriocapillaris, and/or guiding vessel development.

The problem underlying the present invention is also solved in an eighth aspect, which is also a first embodiment of the eighth aspect, by a pigment epithelium-derived factor (PEDF) or an mRNA coding for a pigment epithelium-derived factor (PEDF) for use in a method for treatment and/or prevention of a disease, wherein the method comprises administering the PEDF or the mRNA coding for PEDF to a subject, wherein the disease is corneal neovascularization.

In a second embodiment of the eighth aspect which is also an embodiment of the first embodiment of the eighth aspect, treatment and/or prevention of the disease comprises inhibiting labyrinth capillary formation, inducing growth of choriocapillaris, preferably inducing growth of non-leaking choriocapillaris, tightening choriocapillaris, inhibiting extracellular matrix formation, protecting choriocapillaris, and/or guiding vessel development.

The problem underlying the present invention is also solved in a ninth aspect, which is also a first embodiment of the ninth aspect, by a pigment epithelium-derived factor (PEDF) or an mRNA coding for a pigment epithelium-derived factor (PEDF) for use in a method for treatment and/or prevention of a disease, wherein the method comprises administering the PEDF or the mRNA coding for PEDF to a subject, wherein the disease is polypoidal choroidal vasculopathy.

In a second embodiment of the ninth aspect, which is also an embodiment of the first embodiment of the ninth aspect, treatment and/or prevention of the disease comprises inhibiting labyrinth capillary formation, inducing growth of choriocapillaris, preferably inducing growth of non-leaking choriocapillaris, tightening choriocapillaris, inhibiting extracellular matrix formation, protection choriocapillaris, and/or guiding vessel development.

The problem underlying the present invention is also solved in a tenth aspect, which is also a first embodiment of the tenth aspect, by a pigment epithelium-derived factor (PEDF) or an mRNA coding for a pigment epithelium-derived factor (PEDF) for use in a method for treatment and/or prevention of a disease, wherein the method comprises administering the PEDF or the mRNA coding for PEDF to a subject, wherein the disease is retinopathy of the prematurity.

In a second embodiment of the tenth aspect, which is also an embodiment of the first embodiment of the tenth aspect, treatment and/or prevention of the disease comprises inhibiting labyrinth capillary formation, inducing growth of choriocapillaris, preferably inducing growth of non-leaking choriocapillaris, tightening choriocapillaris, inhibiting extracellular matrix formation, protecting choriocapillaris, and/or guiding vessel development.

The problem underlying the present invention is also solved in an eleventh aspect, which is also a first embodiment of the eleventh aspect, by a pigment epithelium-derived factor (PEDF) or an mRNA coding for a pigment epithelium-derived factor (PEDF) for use in a method for treatment and/or prevention of a disease, wherein the method comprises administering the PEDF or the mRNA coding for PEDF to a subject, wherein the disease is retinal and/or choroidal fibrosis.

In a second embodiment of the eleventh aspect, which is also an embodiment of the first embodiment of the eleventh aspect, treatment and/or prevention of the disease comprises inhibiting labyrinth capillary formation, inducing growth of choriocapillaris, preferably inducing growth of non-leaking choriocapillaris, tightening choriocapillaris, inhibiting extracellular matrix formation, protection choriocapillaris, and/or guiding vessel development.

In a third embodiment of the eleventh aspect, which is also an embodiment of the first and second embodiment of the eleventh aspect, PEDF and/or mRNA coding for PEDF inhibit progression of retinal and/or choroidal fibrosis.

In an embodiment of the first, second, third, fourth, fifth, sixth, seventh, eighth, ninth, tenth, and eleventh aspect, including each and any embodiment thereof, labyrinth capillary formation is labyrinth capillary formation in an eye, preferably in eye disease.

In an embodiment of the first, second, third, fourth, fifth, sixth, seventh, eighth, ninth, tenth, and eleventh aspect, including each and any embodiment thereof, inducing growth of choriocapillaris comprises or is inducing growth of new choriocapillaris, preferably new choriocapillaris are non-leaking choriocapillaris.

In an embodiment of the first, second, third, fourth, fifth, sixth, seventh, eighth, ninth, tenth, and eleventh aspect, including each and any embodiment thereof, inducing growth of choriocapillaris provides choriocapillaris which are capable of replacing original choriocapillaris, preferably original choriocapillaris are diseased choriocapillaris, more preferably choriocapillaris which are capable of replacing original choriocapillaris are non-leaking choriocapillaris.

In an embodiment of the first, second, third, fourth, fifth, sixth, seventh, eighth, ninth, tenth, and eleventh aspect, including each and any embodiment thereof, tightening choriocapillaris comprises tightening pathological choriocapillaris.

In an embodiment of the first, second, third, fourth, fifth, sixth, seventh, eighth, ninth, tenth, and eleventh aspect, including each and any embodiment thereof, inhibiting extracellular matrix formation comprises inhibition of extracellular matrix formation towards the lumen of a blood vessel and/or around a blood vessel.

In an embodiment of the first, second, third, fourth, fifth, sixth, seventh, eighth, ninth, tenth, and eleventh aspect, including each and any embodiment thereof, protecting choriocapillaris comprises protecting choriocapillaris from the damaging effect of an anti-VEGF drug.

In an embodiment of the first, second, third, fourth, fifth, sixth, seventh, eighth, ninth, tenth, and eleventh aspect, including each and any embodiment thereof, protecting choriocapillaris comprises protecting choriocapillaris from the damaging effect of withdrawal of an anti-VEGF drug.

In an embodiment of the first, second, third, fourth, fifth, sixth, seventh, eighth, ninth, tenth, and eleventh aspect, including each and any embodiment thereof, guiding vessel development comprises development of a functional blood vessel, preferably development of a functional blood vessel from a pathological blood vessel.

In an embodiment of the first, second, third, fourth, fifth, sixth, seventh, eighth, ninth, tenth, and eleventh aspect, including each and any embodiment thereof, the pathological blood vessel is the result of a pathological condition, preferably of a pathological condition of the subject, more preferably the pathological condition is the disease from which the subject is suffering or at risk of suffering and/or for the treatment of which PEDF or mRNA coding for PEDF is used or intendent for being used.

In an embodiment of the first, second, third, fourth, fifth, sixth, seventh, eighth, ninth, tenth, and eleventh aspect, including each and any embodiment thereof, PEDF or mRNA coding for PEDF is administered intravitreally or sub-retinally.

In an embodiment of the first, second, third, fourth, fifth, sixth, seventh, eighth, ninth, tenth, and eleventh aspect, including each and any embodiment thereof, the method further comprises applying an anti-VEGF therapy, preferably the anti-VEGF therapy comprises administration to the subject of an anti-VEGF drug, wherein the anti-VEGF drug is selected from the group comprising pegaptanib, ranibizumab, bevacizumab and aflibercept. In an embodiment thereof, the combined use of both PEDF and an anti-VEGF therapy allows the decreasing of the amount of the anti-VEGF therapy administered to the subject compared to the sole use of the anti-VEGF therapy. Such decreasing of the amount of the anti-VEGF therapy administered to the subject typically results in a decrease in side effects, in particular side effects of said anti-VEGF therapy such as cardiovascular side effects and/or cerebral side effects.

Without wishing to be bound by any theory, the present inventor has surprisingly found that pigment epithelium derived factor (PEDF) is capable of inducing growth of healthy and functional choriocapillaris and effects associated therewith such as inhibiting labyrinth capillary formation, tightening choriocapillaris, inhibiting extracellular matrix formation, inducing growth of choriocapillaris, preferably inducing growth of non-leaking choriocapillaris, protecting choriocapillaris and/or guiding vessel development which is beneficial in the treatment of eye diseases. Insofar, the present invention turns away from the current state of the art in the treatment of eye disease which is based on blocking vessel growth or removing vessels.

Additionally, the present inventor has surprisingly found that by balancing the level of PEDF protein and VEGF protein in the eye, various eye diseases, including dry and wet age-related macular degeneration, as well as certain symptoms and morphological changes, respectively, associated with said eye diseases such as treatment-naive quiescent choroidal neovascularization and geographic atrophy, can be treated. In connection therewith it will be acknowledged by a person skilled in the art that such balancing of the level of PEDF protein and VEGF protein may, apart from the administration of PEDF or an mRNA coding for PEDF, require the administration of VEGF or an anti-VEGF agent. Such anti-VEGF agent is an agent which interferes with the activity of VEGF, in particular with the activity of VEGF in an eye, more specifically the angiogenesis activity of VEGF. In an embodiment of each and any aspect of the present invention including any embodiment thereof, such anti-VEGF agent is an anti-VEGF therapy and more specifically an anti-VEGF drug described herein. Methods for determining the level of PEDF protein and VEGF protein in the vitreous body or in the intraocular fluid as a basis for determining whether the level of PEDF and/or VEGF is to be decreased or increased, and if so, to which extent, are known in the art and, for example, described in Biesemeier A et al. (2014).

It will be further acknowledged that in each and any aspect of the present invention, including any embodiment thereof, when referring to the administration of PEDF, the administration of an mRNA coding for PEDF is equally disclosed and encompassed. Similarly, when referring to the administration of VEGF, the administration of an mRNA coding for VEGF is equally disclosed and encompassed. Finally, it will be acknowledged that in each and any aspect of the present invention, including any embodiment thereof, when both PEDF and VEGF are administered to a patient, such administration may encompass (a) the administration of PEDF protein and of VEGF protein, (b) the administration of an mRNA coding for PEDF and of a VEGF protein, (c) the administration of PEDF and of an mRNA coding for VEGF, and (d) the administration of an mRNA coding for PEDF and of an mRNA coding for VEGF.

In light of such inhibition of labyrinth capillary formation by means of PEDF, the therapeutic effectiveness of PEDF in the treatment of eye diseases is plausible, in particular for those eye diseases showing labyrinth capillary formation such as age-related macular degeneration (AMD), both dry AMD and wet AMD, central serous chorioretinopathy, diabetic retinopathy, rubeosis iridis, corneal neovascularization, polypoidal choroidal vasculopathy and retinopathy of the prematurity. For example, if patients with wet AMD are injected with fluorescein it is observed that high amounts of liquid leak out from the pathological vessels in a short time. The most plausible cause for this finding is that there are large gaps between or within the endothelial walls. However, gaps in the endothelium which connect blood cells and extracellular matrix would immediately be closed by thrombocytes which is not happening. Recently, capillaries with many microvilli-like projections of the endothelium which formed a labyrinth-like structure into the vessel's lumen were found in surgically excised choroidal neovascularizations (CNVs) from AMD patients. The lumen of such capillaries showed open connections towards the interstitium. These capillaries were connected to the network of blood vessels because they were filled with plasma and, therefore, they were a source for leakage. This type of capillary was frequently observed in CNVs and was called “labyrinth capillary”. Leaky sites in these labyrinth capillaries cannot be closed by thrombocytes because due to the reduced lumen of the labyrinth capillaries thrombocytes cannot enter. Therefore, this vessel type causes chronic plasma exudation and is the origin of edema (Schraermeyer, Julien et al. 2015).

Furthermore, pigment epithelium-derived factor (PEDF) has a very strong neurotrophic and neuroprotective effect (King and Suzuma 2000). This factor is produced by RPE under normoxic conditions. Production is stopped during hypoxia. This greatly promotes neovascularization. In age-related macular degeneration (AMD), the damaged RPE cells produce too little PEDF. This produces uncontrolled neoangiogenesis. It was believed that the central effect of PEDF in the eye is to prevent neogenesis of vessels (King and Suzuma 2000), but in accordance with the present invention, PEDF can stabilize CNV vessels and avoid Labyrinth capillary formation if pathological vessel formation has been initiated by VEGF.

As already indicated, in a first aspect, which is also a first embodiment of the first aspect, the problem underlying the present invention is solved by a pigment epithelium-derived factor (PEDF) for use in a method for treatment and/or prevention of a disease, wherein the method comprises administering PEDF to a subject and wherein treatment and/or prevention of a disease comprises inhibiting labyrinth capillary formation, inducing growth of choriocapillaris, preferably inducing growth of non-leaking choriocapillaris, tightening choriocapillaris, inhibiting extracellular matrix formation, protecting choriocapillaris, and/or guiding vessel development.

The following embodiments of the first aspect are further embodiments to and of those embodiments of the first aspect recited above. The following numbering of the embodiments of the first aspect specifically refer to the first aspect recited in the immediately preceding paragraph.

In a preferred embodiment thereof and in each and any aspect, including an embodiment thereof, PEDF is human PEDF protein, in a more preferred embodiment, PEDF comprises an amino acid sequence according to SEQ ID NO: 1:

(SEQ ID NO: 1)
QNPASPPEEG SPDPDSTGAL VEEEDPFFKV PVNKLAAAVS
NFGYDLYRVR SSTSPTTNVL LSPLSVATAL SALSLGAEQR
TESIIHRALY YDLISSPDIH GTYKELLDTV TAPQKNLKSA
SRIVFEKKLR IKSSFVAPLE KSYGTRPRVL TGNPRLDLQE
INNWVQAQMK GKLARSTKEI PDEISILLLG VAHFKGQWVT
KFDSRKTSLE DFYLDEERTV RVPMMSDPKA VLRYGLDSDL
SCKIAQLPLT GSMSIIFFLP LKVTQNLTLI EESLTSEFIH
DIDRELKTVQ AVLTVPKLKL SYEGEVTKSL QEMKLQSLFD
SPDFSKITGK PIKLTQVEHR AGFEWNEDGA GTTPSPGLQP
AHLTFPLDYH LNQPFIFVLR DTDTGALLFI GKILDPRGP

In a preferred embodiment thereof and in each and any aspect, including an embodiment thereof, PEDF is a derivative of PEDF, preferably of human PEDF, and more preferably of PEDF comprising an amino acid sequence according to SEQ ID NO: 1. It will be appreciated by a person skilled in the art, that any derivative of PEDF may be used as long as the PEDF is capable of causing the above effects and in particular the effect of inhibiting labyrinth capillary formation, inducing growth of choriocapillaris, tightening choriocapillaris, inhibiting extracellular matrix formation, protecting choriocapillaris, and/or guiding vessel development. In an embodiment, the PEDF is one having a homology or identity to the amino acid sequence of SEQ ID NO: 1 of at least, 85%, 86%, 87%, 88%, 89%, 90%, 91%, 92%, 93%, 94%, 95%, 96%, 97%, 98% or 99%. In a further embodiment, a derivative of PEDF is one where at amino acid position 20 of SEQ ID NO: 1 the amino acid residue is pyrrolidone carboxylic acid, at amino acid position 24 of SEQ ID NO: 1 the amino acid residue is phosphoserine, at amino acid position 114 of SEQ ID NO: 1 the amino acid residue is phosphoserine, at amino acid position 227 of SEQ ID NO: 1 the amino acid residue is phosphoserine and/or at amino acid position 285 of SEQ ID NO: 1 the amino acid residue is N-linked (GlcNAc) asparagine.

It will be appreciated by a person skilled in the art that any of the above effects and, in particular, labyrinth capillary formation and any change thereof including inhibition thereof, induction of growth of choriocapillaris, tightening of choriocapillaris, inhibition of extracellular matrix formation, protection of choriocapillaris, and/or guidance of vessel development can be assessed by optical coherence tomography (OCT-A), preferable when combined with fluorescein angiography (FA) which is suitable for detecting and assessing, respectively, leaking vessels (Spaide et al. 2015). Optical coherence tomography angiography (OCT-A) emerged as a non-invasive technique for imaging the microvasculature of the retina and choroid (Spaide et al.2015). Briefly, OCT-A technology uses laser light reflectance of the surface of moving red blood cells to accurately depict vessels through different segmented areas of the eye, thus eliminating the need for intravascular dyes. The OCT scan of a patient's retina consists in multiple individual A-scans, which compiled into a B-scan provides cross-sectional structural information. With OCT-A technology, the same tissue area is repeatedly imaged and differences analyzed between scans, thus allowing one to detect zones containing high flow rates, i.e. with marked changes between scans, and zones with slower, or no flow at all, which will be similar among scans.

OCT-A and FA may also be used for the detection and assessment, respectively, of edema which are located within the retina and/or subretinal space.

In a second embodiment of the first aspect, which is also an embodiment of the first embodiment of the first aspect, the disease is an eye or ocular disease.

In a third embodiment of the first aspect, which is also an embodiment of the first and second embodiment of the first aspect, the eye disease is macular degeneration, preferably age-related macular degeneration (AMD), more preferably dry age-related macular degeneration or wet age-related macular degeneration.

In a fourth embodiment of the first aspect, which is also an embodiment of the first and second embodiment of the first aspect, the eye disease is selected from the group comprising central serous chorioretinopathy, diabetic retinopathy, rubeosis iridis, corneal neovascularization, polypoidal choroidal vasculopathy and retinopathy of the prematurity.

In a fifth embodiment of the first aspect, which is also an embodiment of the first, second, third and fourth embodiment of the first aspect, labyrinth capillary formation is labyrinth capillary formation in an eye, preferably in eye disease.

In a sixth embodiment of the first aspect, which is also an embodiment of the first, second, third, fourth and fifth embodiment of the first aspect, inducing growth of choriocapillaris comprises or is inducing growth of new choriocapillaris, preferably inducing growth of non-leaking choriocapillaris.

In a seventh embodiment of the first aspect, which is also an embodiment of the first, second, third, fourth, fifth and sixth embodiment of the first aspect, inducing growth of choriocapillaris provides choriocapillaris which are capable of replacing original choriocapillaris, preferably original choriocapillaris are diseased choriocapillaris. In connection therewith, it will be acknowledged by a person skilled in the art that diseased choriocapillaris is located between Bruch's membrane and RPE and can be seen in OCT-A.

In an eight embodiment of the first aspect, which is also an embodiment of the first, second, third, fourth, fifth, sixth and seventh embodiment of the first aspect, tightening choriocapillaris comprises tightening pathological choriocapillaris. In connection therewith, it will be acknowledged that each neovascular choriocapillaris or vessel located between Bruch's membrane and RPE or within the subretinal space is preferably regarded as pathologic. More preferably, a choriocapillaris is regarded as pathologic only when they develop into labyrinthy capillaries or became leaky by other reasons. Diagnosis thereof may be performed by OCT-A and/or fluorescein angiography (FA).

In a ninth embodiment of the first aspect, which is also an embodiment of the first, second, third, fourth, fifth, sixth, seventh and eighth embodiment of the first aspect, inhibiting extracellular matrix formation comprises inhibition of extracellular matrix formation towards the lumen of a blood vessel and/or around a blood vessel. Preferably, such vessel does not inhibit the flow of red blood cells by absence of endothelial projection into the lumen.

In a tenth embodiment of the first aspect, which is also an embodiment of the first, second, third, fourth, fifth, sixth, seventh, eighth and ninth embodiment of the first aspect, protecting choriocapillaris comprises protecting choriocapillaris from the damaging effect of an anti-VEGF drug. Such anti-VEGF drug is preferably one selected from the group comprising pegaptanib, ranibizumab, bevacizumab and aflibercept. In connection therewith, it will be acknowledged that such damaging effect may encompass regression of blood vessels and degeneration of RPE and photoreceptors resulting in geographic atrophy. Geographic atrophy may be detected as a dark spot upon scanning laser ophthalmoscopy (SLO) because autofluorescence of the RPE disappears. The SLO picture is the result of autofluorescence of lipofuscin in RPE.

In an eleventh embodiment of the first aspect, which is also an embodiment of the first, second, third, fourth, fifth, sixth, seventh, eighth, ninth and tenth embodiment of the first aspect, protecting choriocapillaris comprises protecting choriocapillaris from the damaging effect of withdrawal of an anti-VEGF drug. In connection therewith, it will be appreciated that, preferably, blood vessels become leaky and change into labyrinth capillaries; endothelial cells proliferate and migrate.

In a twelfth embodiment of the first aspect, which is also an embodiment of the first, second, third, fourth, fifth, sixth, seventh, eighth, ninth, tenth and eleventh embodiment of the first aspect, guiding vessel development comprises development of a functional blood vessel, preferably development of a functional blood vessel from a pathological blood vessel. In connection therewith, preferably a pathological blood vessel is a blood vessel which does not allow proper blood flow, is leaky of forms too many or atypical extracellular matrix proteins.

In a 13^{th} embodiment of the first aspect, which is also an embodiment of the twelfth embodiment of the first aspect, the pathological blood vessel is the result of a pathological condition. Such pathological condition may be one or a combination of hypoxia, upregulation of HIF 1 alpha, and atypical formation of growth factors and VEGF.

In a 14^{th} embodiment of the first aspect, which is also an embodiment of the first, second, third, fourth, fifth, sixth, seventh, eighth, ninth, tenth, eleventh, twelfth and 13^{th} embodiment of the first aspect, PEDF is administered intravitreally or sub-retinally, or as a vector such as adeno-associated virus coding for PEDF.

In a 15^{th} embodiment of the first aspect, which is also an embodiment of the first, second, third, fourth, fifth, sixth, seventh, eighth, ninth, tenth, eleventh, twelfth, 13^{th} and 14^{th} embodiment of the first aspect, the method further comprises applying an anti-VEGF therapy, preferably the anti-VEGF therapy comprises administration to the subject of an anti-VEGF drug, wherein the anti-VEGF drug is selected from the group comprising pegaptanib, ranibizumab, bevacizumab and aflibercept. In connection therewith, it will be acknowledged by a person skilled in the art that PEDF may be used early, for example when the CNV is detected in one eye, the fellow eye may be treated prophylactically; also, if a subclinical CNV with normal vision of the eye is diagnosed the treatment may begin in order to keep the CNV stable.

In a 16^{th} embodiment of the first aspect, which is also an embodiment of the first, second, third, fourth, fifth, sixth, seventh, eighth, ninth, tenth, eleventh, twelfth, 13^{th}, 14^{th} and 15^{th} embodiment of the first aspect, the subject is a subject who is suffering from side effects of anti-VEGF treatment, preferably visual loss arising from anti-VEGF treatment.

In a second aspect, which is also a first embodiment of the second aspect, the problem underlying the present invention is solved by an mRNA coding for a pigment epithelium-derived factor (PEDF) for use in a method for treatment and/or prevention of a disease, wherein the method comprises administering PEDF to a subject and wherein treatment and/or prevention of a disease comprises inhibiting labyrinth capillary formation, inducing growth of choriocapillaris, tightening choriocapillaris, inhibiting extracellular matrix formation, protecting choriocapillaris, and/or guiding vessel development. In an embodiment, the mRNA is an mRNA coding for the amino acid sequence according to SEQ ID NO: 1. It is appreciated by a person skilled in the art that if an mRNA coding for PEDF is used in accordance with the present invention, such as in a method for treatment and/or prevention of a disease, the mRNA contains a sequence that preferably codes for a signal peptide that directs the mRNA into the endoplasmic reticulum (ER) and that is subsequently cleaved off. In an embodiment, the mRNA is an mRNA coding for the amino acid sequence according to SEQ ID NO: 1:

ATGCAGGCCCTGGTGCTACTCCTCTGCATTGGAGCCCTCCTCGGGCACA
GCAGCTGCCAGAACCCTGCCAGCCCCCCGGAGGAGGGCTCCCCAGACCC
CGACAGCACAGGGGCGCTGGTGGAGGAGGAGGATCCTTTCTTCAAAGTC
CCCGTGAACAAGCTGGCAGCGGCTGTCTCCAACTTCGGCTATGACCTGT
ACCGGGTGCGATCCAGCACGAGCCCCACGACCAACGTGCTCCTGTCTCC
TCTCAGTGTGGCCACGGCCCTCTCGGCCCTCTCGCTGGGAGCGGAGCAG
CGAACAGAATCCATCATTCACCGGGCTCTCTACTATGACTTGATCAGCA
GCCCAGACATCCATGGTACCTATAAGGAGCTCCTTGACACGGTCACCGC
CCCCCAGAAGAACCTCAAGAGTGCCTCCCGGATCGTCTTTGAGAAGAAG
CTGCGCATAAAATCCAGCTTTGTGGCACCTCTGGAAAAGTCATATGGGA
CCAGGCCCAGAGTCCTGACGGGCAACCCTCGCTTGGACCTGCAAGAGAT
CAACAACTGGGTGCAGGCGCAGATGAAAGGGAAGCTCGCCAGGTCCACA
AAGGAAATTCCCGATGAGATCAGCATTCTCCTTCTCGGTGTGGCGCACT
TCAAGGGGCAGTGGGTAACAAAGTTTGACTCCAGAAAGACTTCCCTCGA
GGATTTCTACTTGGATGAAGAGAGGACCGTGAGGGTCCCCATGATGTCG
GACCCTAAGGCTGTTTTACGCTATGGCTTGGATTCAGATCTCAGCTGCA
AGATTGCCCAGCTGCCCTTGACCGGAAGCATGAGTATCATCTTCTTCCT
GCCCCTGAAAGTGACCCAGAATTTGACCTTGATAGAGGAGAGCCTCACC
TCCGAGTTCATTCATGACATAGACCGAGAACTGAAGACCGTGCAGGCGG
TCCTCACTGTCCCCAAGCTGAAGCTGAGTTACGAAGGCGAAGTCACCAA
GTCCCTGCAGGAGATGAAGCTGCAATCCTTGTTTGATTCACCAGACTTT
AGCAAGATCACAGGCAAACCCATCAAGCTGACTCAGGTGGAACACCGGG
CTGGCTTTGAGTGGAACGAGGATGGGGCGGGAACCACCCCCAGCCCAGG
GCTGCAGCCTGCCCACCTCACCTTCCCGCTGGACTATCACCTTAACCAG
CCTTTCATCTTCGTACTGAGGGACACAGACACAGGGGCCCTTCTCTTCA
TTGGCAAGATTCTGGACCCCAGGGGCCCCTAA.

The first 57 nucleotides of the nucleotide sequence of SEQ ID NO: 2 code for the signal peptide of the human PEDF. It is, however, within the present invention that said signal peptide and the nucleotide sequence coding therefor, is replaced by a different signal peptide and the nucleotide sequence coding for such different signal peptide, respectively. Such different signal peptides are known in the art.

In an alternative embodiment, the mRNA is a nucleotide sequence of SEQ ID NO: 3:

(SEQ ID NO: 3)
GGACGCTGGATTAGAAGGCAGCAAAAAAAGATCTGTGCTGGCTGGAGCC
CCCTCAGTGTGCAGGCTTAGAGGGACTAGGCTGGGTGTGGAGCTGCAGC
GTATCCACAGGCCCCAGGATGCAGGCCCTGGTGCTACTCCTCTGCATTG
GAGCCCTCCTCGGGCACAGCAGCTGCCAGAACCCTGCCAGCCCCCCGGA
GGAGGGCTCCCCAGACCCCGACAGCACAGGGGCGCTGGTGGAGGAGGAG
GATCCTTTCTTCAAAGTCCCCGTGAACAAGCTGGCAGCGGCTGTCTCCA
ACTTCGGCTATGACCTGTACCGGGTGCGATCCAGCATGAGCCCCACGAC
CAACGTGCTCCTGTCTCCTCTCAGTGTGGCCACGGCCCTCTCGGCCCTC
TCGCTGGGAGCGGACGAGCGAACAGAATCCATCATTCACCGGGCTCTCT
ACTATGACTTGATCAGCAGCCCAGACATCCATGGTACCTATAAGGAGCT
CCTTGACACGGTCACTGCCCCCCAGAAGAACCTCAAGAGTGCCTCCCGG
ATCGTCTTTGAGAAGAAGCTRCGCATAAAATCCAGCTTTGTGGCACCTC
TGGAAAAGTCATATGGGACCAGGCCCAGAGTCCTGACGGGCAACCCTCG
CTTGGACCTGCAAGAGATCAACAACTGGGTGCAGGCGCAGATGAAAGGG
AAGCTCGCCAGGTCCACAAAGGAAATTCCCGATGAGATCAGCATTCTCC
TTCTCGGTGTGGCGCACTTCAAGGGGCAGTGGGTAACAAAGTTTGACTC
CAGAAAGACTTCCCTCGAGGATTTCTACTTGGATGAAGAGAGGACCGTG
AGGGTCCCCATGATGTCGGACCCTAAGGCTGTTTTACGCTATGGCTTGG
ATTCAGATCTCAGCTGCAAGATTGCCCAGCTGCCCTTGACCGGAAGCAT
GAGTATCATCTTCTTCCTGCCCCTGAAAGTGACCCAGAATTTGACCTTG
ATAGAGGAGAGCCTCACCTC

In a further embodiment of the second aspect, including any embodiment thereof, the mRNA is a recombinant or heterologous mRNA which preferably comprises a structural element such as a 5′ UTR and/or a 3′ UTR, which is different from the 5′ UTR and/or the 3′ UTR of the mRNA from which the coding sequence of PEDF is taken.

The disclosure of the first aspect, including any embodiment thereof, equally applies to the second aspect, including any embodiment thereof. In other words, each and any embodiment of the first aspect is also an embodiment of the second aspect, including any embodiment thereof, and vice versa.

In a twelfth aspect, which is also a first embodiment of the twelfth aspect, the problem underlying the present invention is also solved by a pigment epithelium-derived factor (PEDF) for use in a method for the treatment and/or prevention of dry macular degeneration in a subject, preferably dry age-related macular degeneration, wherein the method comprises administering PEDF and VEGF to the subject.

In a second embodiment of the twelfth aspect, which is also an embodiment of the first embodiment of the twelfth aspect, the subject is suffering from treatment-naïve quiescent choroidal neovascularization. In connection with this embodiment it is to be acknowledged that treatment-naïve quiescent choroidal neovascularization is observed in individuals between the choroid and the retinal pigment epithelium with the absence of clinical symptoms. These CNV's are fully perfused but lack any leakage and exudation of liquid. Also, the visual acuity in these individuals can be normal. (See, Querques et al., and Mentes J and Yildirim S).

In a third embodiment of the twelfth aspect, which is also an embodiment of the first and second embodiment of the twelfth aspect, the visual acuity of the subject is normal.

In a fourth embodiment of the twelfth aspect, which is also an embodiment of the first, second and third embodiment of the twelfth aspect, wherein the subject is suffering from geographic atrophy. In connection with this embodiment, it is to be acknowledged that geographic atrophy (GA) represents the nonexudative late stage of age-related macular degeneration (AMD). It is typically characterized by areas of loss of outer retinal layers including photoreceptors, degeneration of the retinal pigment epithelium, and rarefication of the choriocapillaris. (See, Muller, P. L., et al. (2020)).

In a fifth embodiment of the twelfth aspect, which is also an embodiment of the fourth embodiment of the twelfth aspect, geographic atrophy is the result of anti-VEGF therapy.

In a sixth embodiment of the twelfth aspect, which is also an embodiment of the first, second, third, fourth and fifth embodiment of the twelfth aspect, PEDF induces growth of choriocapillaris.

In a seventh embodiment of the twelfth aspect, which is also an embodiment of the sixth embodiment of the twelfth aspect, choriocapillaris are non-leaking choriocapillaris.

In an eighth embodiment of the twelfth aspect, which is also an embodiment of the first, second, third, fourth, fifth, sixth and seventh embodiment of the twelfth aspect, PEDF inhibits growth and/or formation of geographic atrophy.

It is to be acknowledged that the disclosure of the first aspect, including any embodiment thereof, equally applies to the twelfth aspect. In other words, each and any embodiment of the first aspect is also an embodiment of the twelfth aspect, including any embodiment thereof, and vice versa.

In a 13^{th} aspect, which is also a first embodiment of the 13^{th} aspect, the problem underlying the present invention is also solved by an mRNA coding for pigment epithelium-derived factor (PEDF) for use in a method for the treatment and/or prevention of dry macular degeneration in a subject, preferably dry age-related macular degeneration, wherein the method comprises administering the mRNA coding for PEDF and VEGF or an mRNA coding for VEGF to the subject.

It is to be acknowledged that the disclosure of the second and twelfth aspect, including any embodiment thereof, equally applies to the 13^{th} aspect. In other words, each and any embodiment of the second aspect and each and any embodiment of the twelfth aspect is also an embodiment of the 13^{th} aspect, including any embodiment thereof, and vice versa.

In a 14^{th} aspect, which is also a first embodiment of the 14^{th} aspect, the problem underlying the present invention is also solved by a pigment epithelium-derived factor (PEDF) for use in a method for the treatment and/or prevention of wet macular degeneration in a subject, preferably wet age-related macular degeneration, wherein the method comprises administering PEDF and an anti-VEGF therapy to the subject.

In a second embodiment of the 14^{th} aspect, which is also an embodiment of the first embodiment of the 14^{th} aspect, PEDF induces growth of non-leaking choriocapillaris.

In a third embodiment of the 14^{th} aspect, which is also an embodiment of the first and second embodiment of the 14^{th} aspect, the anti-VEGF therapy comprises administration to the subject of an anti-VEGF drug.

In a fourth embodiment of the 14^{th} aspect, which is also an embodiment of the first, second and third embodiment of the 14^{th} aspect, the anti-VEGF drug is selected from the group comprising pegaptanib, ranibizumab, bevacizumab and aflibercept.

It is to be acknowledged that the disclosure of the first aspect, including each and any embodiment thereof, equally applies to the 14^{th} aspect. In other words, each and any embodiment of the first aspect is also an embodiment of the 14^{th} aspect, including any embodiment thereof, and vice versa.

In a 15^{th} aspect, which is also a first embodiment of the 15^{th} aspect, the problem underlying the present invention is also solved by an mRNA coding for pigment epithelium-derived factor (PEDF) for use in a method for the treatment and/or prevention of wet macular degeneration in a subject, preferably wet age-related macular degeneration, wherein the method comprises administering the mRNA coding for PEDF and an anti-VEGF therapy to the subject.

It is to be acknowledged that the disclosure of the second and 14^{th} aspect, including any embodiment thereof, equally applies to the 15^{th} aspect. In other words, each and any embodiment of the second aspect and each and any embodiment of the 14^{th} aspect is also an embodiment of the 15^{th} aspect, including any embodiment thereof, and vice versa.

In a 16^{th} aspect, which is also a first embodiment of the 16^{th} aspect, the problem underlying the present invention is also solved by a pigment epithelium-derived factor (PEDF) for use in a method for the treatment and/or prevention of treatment-naïve quiescent choroidal neovascularization in a subject, wherein the method comprises administering PEDF and an VEGF to the subject.

In a second embodiment of the 16^{th} aspect, which is also an embodiment of the first embodiment of the 16^{th} aspect, the visual acuity of the subject is normal.

In a third embodiment of the 16^{th} aspect, which is also an embodiment of the first and second embodiment of the 16^{th} aspect, the subject is suffering from dry macular degeneration, preferably dry age-related macular degeneration.

In a fourth embodiment of the 16^{th} aspect, which is also an embodiment of the first, second and third embodiment of the 16^{th} aspect, the subject is suffering from geographic atrophy.

In a fifth embodiment of the 16^{th} aspect, which is also an embodiment of the fourth embodiment of the 16^{th} aspect, geographic atrophy is the result of anti-VEGF therapy.

In a sixth embodiment of the 16^{th} aspect, which is also an embodiment of the first, second, third, fourth and fifth embodiment of the 16^{th} aspect, PEDF induces growth of choriocapillaris.

In a seventh embodiment of the 16^{th} aspect, which is also an embodiment of the sixth embodiment of the 16^{th} aspect, choriocapillaris are non-leaking choriocapillaris.

It is to be acknowledged that the disclosure of the first aspect, including any embodiment thereof, equally applies to the 16^{th} aspect. In other words, each and any embodiment of the first aspect is also an embodiment of the 16^{th} aspect, including any embodiment thereof, and vice versa.

In a 17^{th} aspect, which is also a first embodiment of the 17^{th} aspect, the problem underlying the present invention is also solved by an mRNA coding for pigment epithelium-derived factor (PEDF) for use in a method for the treatment and/or prevention of treatment-naïve quiescent choroidal neovascularization in a subject, wherein the method comprises administering the mRNA coding for PEDF and VEGF or an mRNA coding for VEGF to the subject.

It is to be acknowledged that the disclosure of the second and 16^{th} aspect, including any embodiment thereof, equally applies to the 17^{th} aspect. In other words, each and any embodiment of the second aspect and each and any embodiment of the 16^{th} aspect is also an embodiment of the 17^{th} aspect, including any embodiment thereof, and vice versa.

In an 18^{th} aspect which is also a first embodiment of the 18^{th} aspect, the problem underlying the present invention is solved by a pigment epithelium-derived factor (PEDF) for use in a method for the treatment and/or prevention of subclinical neovascularization in a subject, wherein the method comprises administering PEDF or an mRNA coding for PEDF to the subject.

In a second embodiment of the 18^{th} aspect, which is also an embodiment of the first embodiment of the 18^{th} aspect, the subject is suffering from dry macular degeneration, preferably dry age-related macular degeneration.

In a third embodiment of the 18^{th} aspect, which is also an embodiment of the first and second embodiment of the 18^{th} aspect, PEDF or the mRNA coding for PEDF induces growth of choriocapillaris, preferably induces growth of non-leaking choriocapillaris.

In a fourth embodiment of the 18^{th} aspect, which is also an embodiment of the first, second and third embodiment of the 18^{th} aspect, the method further comprises administering to the subject VEGF, an mRNA coding for VEGF, and/or an anti-VEGF drug.

In a fifth embodiment of the 18^{th} aspect, which is also an embodiment of the first embodiment of the 18^{th} aspect, the subject is suffering from wet macular degeneration, preferably wet age-related macular degeneration.

In a 19^{th} aspect which is also a first embodiment of the 19^{th} aspect, the problem underlying the present invention is solved by a pigment epithelium-derived factor (PEDF) or an mRNA coding for PEDF for use in a method for the treatment and/or prevention of macular degeneration in a subject, wherein the subject is suffering from subclinical neovascularization.

In a second embodiment of the 19^{th} aspect, which is also an embodiment of the first embodiment of the 19^{th} aspect, macular degeneration is age-related macular degeneration.

In a third embodiment of the 19^{th} aspect, which is also an embodiment of the first and the second embodiment of the 19^{th} aspect, macular degeneration is dry macular degeneration.

In a fourth embodiment of the 19^{th} aspect, which is also an embodiment of third embodiment of the 19^{th} aspect, the method further comprises administering to the subject a VEGF or an mRNA coding for VEGF.

In a fifth embodiment of the 19^{th} aspect, which is also an embodiment of the first, second, third and fourth embodiment of the 19^{th} aspect, PEDF or the mRNA coding for PEDF induces growth of choriocapillaris, preferably growth of non-leaking choriocapillaris.

In a sixth embodiment of the 19^{th} aspect which is also an embodiment of the first and the second embodiment of the 19^{th} aspect, macular degeneration is wet macular degeneration.

It is to be acknowledged that the disclosure of the first and second aspect, including any embodiment thereof, equally applies to the 18^{th} aspect and 19^{th} aspect including any embodiment thereof. In other words, each and any embodiment of the first aspect and the second aspect is also an embodiment of the 18^{th} and 19^{th} aspect, including any embodiment thereof, and vice versa.

In a 20^{th} aspect, which is also a first embodiment of the 20^{th} aspect, the problem underlying the present invention is also solved by a pharmaceutical composition either comprising a pigment epithelium-derived factor (PEDF) or an mRNA coding for a pigment epithelium-derived factor (PEDF), wherein the pharmaceutical composition is for use in a method for treatment and/or prevention of a disease, wherein the method comprises administering PEDF or an mRNA coding for PEDF to a subject and wherein treatment and/or prevention of a disease comprises inhibiting labyrinth capillary formation, inducing growth of choriocapillaris, preferably inducing growth of non-leaking choriocapillaris, tightening choriocapillaris, inhibiting extracellular matrix formation, protecting choriocapillaris, and/or guiding vessel development. Preferably, the pharmaceutical composition comprises a pharmaceutically acceptable excipient or diluent.

The disclosure of the first aspect, the second aspect, the twelfth aspect, 13^{th} aspect, 14^{th} aspect, 15^{th} aspect, 16^{th} aspect, 17^{th}, 18^{th} and 19^{th} aspect, including any embodiment thereof, equally applies to the 20^{th} aspect, including any embodiment thereof. In other words, each and any embodiment of the first aspect, the second aspect, the twelfth aspect, 13^{th} aspect, 14^{th} aspect, 15^{th} aspect, 16^{th} aspect, 17^{th} aspect, 18^{th} aspect and 19^{th} aspect, including any embodiment thereof, is also an embodiment of the 20^{th} aspect including any embodiment thereof, and vice versa.

In a 21^{st} aspect which is also a first embodiment of the 21^{st} aspect, the problem underlying the present invention is also solved by a pharmaceutical composition either comprising a pigment epithelium-derived factor (PEDF) or an mRNA coding for a pigment epithelium-derived factor (PEDF), wherein the pharmaceutical composition is for use in a method for treatment and/or prevention of a disease, wherein the disease is an eye disease.

The disclosure of the third, fourth, fifth, sixth, seventh, eighth, ninth, tenth, eleventh, twelfth, 13^{th}, 14^{th}, 15^{th}, 16^{th}, 17^{th}, 18^{th}, 19^{th} and 20^{th} aspect, including any embodiment thereof, equally applies to the 21^{st} aspect, including any embodiment thereof. In other words, each and any embodiment of the third, fourth, fifth, sixth, seventh, eighth, ninth, tenth, eleventh, twelfth, 13^{th}, 14^{th}, 15^{th}, 16^{th}, 17^{th}, 18^{th}, 19^{th} and 20^{th} aspect, including any embodiment thereof, is also an embodiment of the 21^{st} aspect including any embodiment thereof, and vice versa.

In a 22^{nd} aspect, which is also a first embodiment of the 22^{nd} aspect, the problem underlying the present invention is solved by the use of a pigment epithelium-derived factor (PEDF) or an mRNA coding for a pigment epithelium-derived factor (PEDF) for the manufacture of a medicament for the treatment and/or prevention of a diseases, wherein treatment and/or prevention of a disease comprises inhibiting labyrinth capillary formation, inducing growth of choriocapillaris, preferably inducing growth of non-leaking choriocapillaris, tightening choriocapillaris, inhibiting extracellular matrix formation, protecting choriocapillaris, and/or guiding vessel development.

The disclosure of the first aspect, the second aspect, the twelfth, 13^{th} aspect, 14^{th} aspect, 15^{th} aspect, 16^{th} aspect, 17^{th} aspect, 18^{th} aspect and 19^{th} aspect, including any embodiment thereof, equally applies to the 22^{nd} aspect including any embodiment thereof. In other words, any embodiment of the first aspect, the second aspect, the twelfth, 13^{th} aspect, 14^{th} aspect, 15^{th} aspect, 16^{th} aspect, 17^{th} aspect, 18^{th} aspect and 19^{th} aspect, is also an embodiment of the 22^{nd} aspect including any embodiment thereof, and vice versa.

In a 23^{rd} aspect which is also a first embodiment of the 23^{rd} aspect, the problem underlying the present invention is also solved by the use of a pigment epithelium-derived factor (PEDF) or an mRNA coding for a pigment epithelium-derived factor (PEDF) for the manufacture of a medicament for the treatment and/or prevention of a disease, wherein the disease is an eye disease.

The disclosure of the third, fourth, fifth, sixth, seventh, eighth, ninth, tenth, eleventh, twelfth, 13^{th}, 14^{th}, 15^{th}, 16^{th}, 17^{th}, 18^{th} and 19^{th} aspect, including any embodiment thereof, equally applies to the 23^{rd} aspect, including any embodiment thereof. In other words, each and any embodiment of the third, fourth, fifth, sixth, seventh, eighth, ninth, tenth, eleventh, twelfth, 13^{th}, 14^{th}, 15^{th}, 16^{th}, 17^{th}, 18^{th} and 19^{th} aspect, including any embodiment thereof, is also an embodiment of the 23^{rd} aspect including any embodiment thereof, and vice versa.

In a 24^{th} aspect, which is also a first embodiment of the 24^{th} aspect, the problem underlying the present invention is solved by a method for the treatment and/or prevention of a disease in a subject, wherein treatment and/or prevention of a disease comprises administering to the subject a therapeutically effective amount of a pigment epithelium-derived factor (PEDF) or an mRNA coding for a pigment epithelium-derived factor (PEDF) and inhibiting labyrinth capillary formation, inducing growth of choriocapillaris, preferably inducing growth of non-leaking choriocapillaris, tightening choriocapillaris, inhibiting extracellular matrix formation, protecting choriocapillaris, and/or guiding vessel development.

The disclosure of the first aspect, second aspect, the twelfth, 13^{th} aspect, 14^{th} aspect, 15^{th} aspect, 16^{th} aspect, 17^{th} aspect, 18^{th} and 19^{th} aspect, including any embodiment thereof, equally applies to the 24^{th} aspect, including any embodiment thereof. In other words, any embodiment of the first aspect, the second aspect, the twelfth, 13^{th} aspect, 14^{th} aspect, 15^{th} aspect, 16^{th} aspect, 17^{th}, 18^{th} and 19^{th} aspect, is also an embodiment of the 24^{th} aspect including any embodiment thereof, and vice versa.

In a 25^{th} aspect which is also a first embodiment of the 25^{th} aspect, the problem underlying the present invention is also solved by a method for the treatment and/or prevention of a disease in a subject, wherein treatment and/or prevention of a disease comprises administering to the subject a therapeutically effective amount of a pigment epithelium-derived factor (PEDF) or an mRNA coding for a pigment epithelium-derived factor (PEDF), wherein the disease is an eye disease.

The disclosure of the third, fourth, fifth, sixth, seventh, eighth, ninth, tenth, eleventh, twelfth, 13^{th}, 14^{th}, 15^{th}, 16^{th}, 17^{th}, 18^{th} and 19^{th} aspect, including any embodiment thereof, equally applies to the 25^{th} aspect, including any embodiment thereof. In other words, each and any embodiment of the third, fourth, fifth, sixth, seventh, eighth, ninth, tenth, eleventh, twelfth, 13^{th}, 14^{th}, 15^{th}, 16^{th}, 17^{th}, 18^{th} and 19^{th} aspect, including any embodiment thereof, is also an embodiment of the 25^{th} aspect including any embodiment thereof, and vice versa. In a preferred embodiment, the treatment and/or prevention of the disease comprises inhibiting labyrinth capillary formation, inducing growth of choriocapillaris, preferably inducing growth of non-leaking choriocapillaris, tightening choriocapillaris, inhibiting extracellular matrix formation, protecting choriocapillaris, and/or guiding vessel development.

In an 26^{th} aspect, which is also a first embodiment of the 26^{th} aspect, the problem underlying the present invention is solved by a pigment epithelium-derived factor (PEDF) for use, in a subject, in a method for inhibiting labyrinth capillary formation, inducing growth of choriocapillaris, preferably inducing growth of non-leaking choriocapillaris, tightening choriocapillaris, inhibiting extracellular matrix formation, protecting choriocapillaris, and/or guiding vessel development, and wherein the method comprises administering PEDF to the subject.

The disclosure of the first aspect, the second aspect, the twelfth, 13^{th} aspect, 14^{th} aspect, 15^{th} aspect, 16^{th} aspect, 17^{th} aspect, 18^{th} aspect and 19^{th} aspect, including any embodiment thereof, equally applies to the 26^{th} aspect including any embodiment thereof. In other words, any embodiment of the first aspect, the second aspect, the twelfth, 13^{th} aspect, 14^{th} aspect, 15^{th} aspect, 16^{th} aspect, 17^{th} aspect, 18^{th} aspect and 19^{th} is also an embodiment of the 26^{th} aspect, including any embodiment thereof, and vice versa.

In a 27^{th} aspect, which is also a first embodiment of the 27^{th} aspect, the problem underlying the present invention is solved by an mRNA coding for a pigment epithelium-derived factor (PEDF) pigment epithelium-derived factor (PEDF) for use, in a subject, in a method for inhibiting labyrinth capillary formation, inducing growth of choriocapillaris, preferably inducing growth of non-leaking choriocapillaris, tightening choriocapillaris, inhibiting extracellular matrix formation, protecting choriocapillaris, and/or guiding vessel development, and wherein the method comprises administering PEDF to the subject.

The disclosure of the first aspect, second aspect, the twelfth, 13^{th} aspect, 14^{th} aspect, 15^{th} aspect, 16^{th} aspect, 17^{th} aspect, 18^{th} aspect and 19^{th} aspect, including any embodiment thereof, equally applies to the 27^{th} aspect. In other words, any embodiment of the first and second aspect is also an embodiment of the 27^{th} aspect including any embodiment thereof, and vice versa.

In an embodiment of each and any aspect, including any embodiment thereof, the subject is a human subject.

In connection with each and any aspect, including any embodiment thereof, VEGF is human VEGF.

In connection with each and any aspect, including any embodiment thereof, preferably the amount of PEDF or of an mRNA coding for PEDF is a therapeutically effective amount thereof.

In connection with each and any aspect, including any embodiment thereof, preferably the amount of VEGF or of an mRNA coding for VEGF is a therapeutically effective amount thereof.

In connection with each and any aspect, including any embodiment thereof, the mRNA coding for PEDF may actually be expressed from an insert, commonly as a cDNA insert, in a suitable non-viral vector or in a viral vector such as an Adeno-associated virus (AAV). If expressed from a non-viral or a viral vector, the insert coding for PEDF is preferably under transcriptional control by a constitutive or a regulatable promoter.

In connection with each and any aspect, including any embodiment thereof, the mRNA coding for VEGF may actually be expressed from an insert, commonly as a cDNA insert, in a suitable non-viral vector or in a viral vector such as an Adeno-associated virus (AAV). If expressed from a non-viral or a viral vector, the insert coding for VEGF is preferably under transcriptional control by a constitutive or a regulatable promoter.

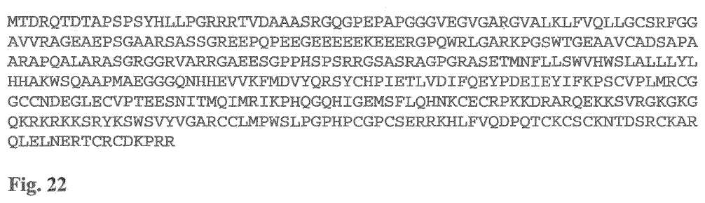

In connection with each and any aspect, including any embodiment thereof, human VEGF is selected from the group comprising human VEGF-A, VEGF-B, VEGF-C, VEGF-D and any isoform thereof. Preferred embodiments of the amino acid sequence and of the mRNA coding for VEGF-A, VEGF-B, VEGF-C and VEGF-D, including the various isoforms thereof, are disclosed in FIGS. 22 to 65. It will be acknowledged by a person skilled in the art that the mRNA sequences are actually depicted as cDNA sequences from which the corresponding mRNA sequences can be derived by replacing T by U.

In an embodiment of each and any aspect and as preferably used herein, labyrinth capillary formation is labyrinth capillary formation in an eye, preferably in eye disease.

In an embodiment of each and any aspect and as preferably used herein, inducing growth of choriocapillaris comprises or is inducing growth of new choriocapillaris.

In an embodiment of each and any aspect and as preferably used herein, inducing growth of choriocapillaris provides choriocapillaris which are capable of replacing original choriocapillaris, preferably original choriocapillaris are diseased choriocapillaris.

In an embodiment of each and any aspect and as preferably used herein, tightening choriocapillaris comprises tightening pathological choriocapillaris.

In an embodiment of each and any aspect and as preferably used herein, inhibiting extracellular matrix formation comprises inhibition of extracellular matrix formation towards the lumen of a blood vessel and/or around a blood vessel.

In an embodiment of each and any aspect and as preferably used herein, protecting choriocapillaris comprises protecting choriocapillaris from the damaging effect of an anti-VEGF drug.

In an embodiment of each and any aspect and as preferably used herein, protecting choriocapillaris comprises protecting choriocapillaris from the damaging effect of withdrawal of an anti-VEGF drug.

In an embodiment of each and any aspect and as preferably used herein, guiding vessel development comprises development of a functional blood vessel, preferably a functional blood vessel from a pathological blood vessel.

In an embodiment of each and any aspect and as preferably used herein, PEDF is human PEDF.

In an embodiment of each and any aspect and as preferably used herein, non-leaking choriocapillaris is choriocapillaris the leaking characteristics of which correspond to the leaking characteristics of a healthy subject, preferably of a healthy human subject.

In an embodiment of each and any aspect of the present invention, including any embodiment thereof, and as preferably used herein, the subject to which the PEDF and/or the mRNA coding for PEDF is a subject suffering from the indicated disease.

In an embodiment of each and any aspect of the present invention, including any embodiment thereof, the term anti-VEGF drug comprises any anti-VEGF therapy.

In an embodiment of each and any aspect of the present invention, including any embodiment thereof, anti-VEGF treatment comprises administering to a subject, preferably a subject in need thereof, an anti-VEGF therapy, preferably an anti-VEGF drug.

It will be appreciated by a person skilled in the art that any embodiment of any aspect, including each and any embodiment thereof, is also an embodiment of each and any one of the other aspects, including any embodiment thereof.

It will be further appreciated by a person skilled in the art that the embodiments of the first aspect recited for the first time constitute an embodiment of the first aspect recited for the second time.

It will also be appreciated that labyrinth capillary formation is a means for defining a group of patients and thus a clinical setting which can be treated by means of the present invention, more specifically due to the treatment in accordance with the present invention such labyrinth capillary formation is inhibited.

It will also be appreciated that little or missing growth of choriocapillaris, preferably growth of non-leaking choriocapillaris is a means for defining a group of patients and thus a clinical setting which can be treated by means of the present invention, more specifically due to the treatment in accordance with the present invention growth of choriocapillaris, preferably growth of non-leaking choriocapillaris is induced.

It will also be appreciated that the presence of non-tight choriocapillaris is a means for defining a group of patients and thus a clinical setting which can be treated by means of the present invention, more specifically due to the treatment in accordance with the present invention such non-tight choriocapillaris are tightened.

It will also be appreciated that abundant extracellular matrix formation is a means for defining a group of patients and thus a clinical setting which can be treated by means of the present invention, more specifically due to the treatment in accordance with the present invention such abundant extracellular matrix formation is inhibited.

It will also be appreciated that non-protected choriocapillaris is a means for defining a group of patients and thus a clinical setting which can be treated by means of the present invention, more specifically due to the treatment in accordance with the present invention such non-protected choriocapillaris are protected.

It will also be appreciated that unguided vessel development is a means for defining a group of patients and thus a clinical setting which can be treated by means of the present invention, more specifically due to the treatment in accordance with the present invention such unguided vessel development can be converted into guided vessel development.

Preferably, subclinical choroidal neovascularization (subclinical CNV) is detected and diagnosed by optical coherence tomography angiography (see, e.g., Treister A 2018).

Subclinical choroidal neovascularization (subclinical CNV) has originally been described by Green W et al. (Trans Am Ophthalmol Soc. 1977; 75: 180) and Sarks S et al. (Br J Ophthalmol 1973, 57:951) and is characterized by abnormal choroidal vessels passing through breaks in the Bruch's membrane in postmortem eyes that had not overlying hemorrhage or exudation. Yannuzzi et al. (Retina, 1992; 12:191-223) showed that indocyanine-green angiography (ICGA) could be used to detect subclinical CNV lesions as hyperfluorescence with late staining of choroidal vessels. ICGA was performed in conjunction with fluorescence angiography (FA); however, FA was shown to be limited by lack of penetration into the choroid as well as more leakage due to lower affinity for large proteins.

It will be appreciated that a pharmaceutical composition comprises at least PEDF or an mRNA coding for PEDF and preferably a pharmaceutically acceptable excipient. Such excipient can be any excipient used and/or known in the art. More particularly such excipient is any excipient as discussed in connection with the manufacture of the medicament disclosed herein. In a further embodiment, the pharmaceutical composition comprises a further pharmaceutically active agent.

The preparation of a medicament and a pharmaceutical composition is known to a person skilled in the art in light of the present disclosure. Typically, such compositions may be prepared as injectables, either as liquid solutions or suspensions; solid forms suitable for solution in, or suspension in, liquid prior to injection; as tablets or other solids for oral administration; as time release capsules; or in any other form currently used, including eye drops, creams, lotions, salves, inhalants and the like. The use of sterile formulations, such as saline-based washes, by surgeons, physicians or health care workers to treat a particular area in the operating field may also be particularly useful. Compositions may also be delivered via microdevice, microparticle or sponge.

Upon formulation, a medicament will be administered in a manner compatible with the dosage formulation, and in such amount as is pharmacologically effective. The formulations are easily administered in a variety of dosage forms, such as the type of injectable solutions described above, but drug release capsules and the like can also be employed.

In this context, the quantity of active ingredient and volume of composition to be administered depends on the individual or the subject to be treated. Specific amounts of active compound required for administration depend on the judgment of the practitioner and are peculiar to each individual.

A minimal volume of a medicament required to disperse the active compounds is typically utilized. Suitable regimes for administration are also variable, but would be typified by initially administering the compound and monitoring the results and then giving further controlled doses at further intervals.

The pharmaceutical composition or medicament may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating, or coating methods, and typically contain about 0.1% to 75%, preferably about 1% to 50%, of the active ingredient.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in liquid prior to injection can be formulated.

If desired, the pharmaceutical composition and medicament, respectively, to be administered may also contain minor amounts of non-toxic auxiliary substances such as wetting or emulsifying agents, pH buffering agents, and other substances such as for example, sodium acetate, and triethanolamine oleate.

The dosage regimen utilizing the nucleic acid molecules and medicaments, respectively, of the present invention is selected in accordance with a variety of factors including type, species, age, weight, sex and medical condition of the patient; the severity of the condition to be treated; the route of administration; the renal and hepatic function of the patient; and the particular aptamer or salt thereof employed. An ordinarily skilled physician or veterinarian can readily determine and prescribe the effective amount of the drug required to prevent, counter or arrest the progress of the condition.

The present invention is further illustrated by the following figures, examples and sequence listing from which further features, embodiment and advantages may be taken. In connection therewith,

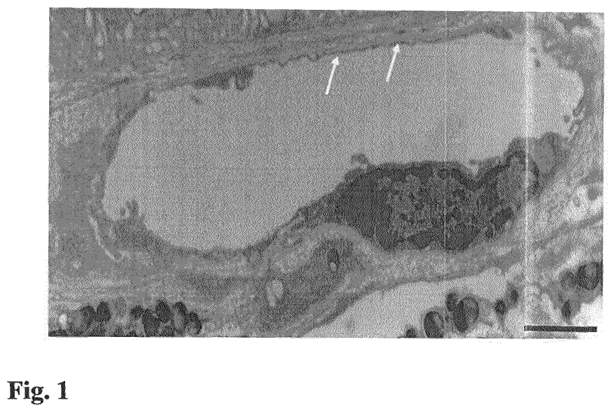

FIG. 1 is an electron micrograph showing a choriocapillaris of an untreated rat that was fixed directly after enucleation; the arrows mark the fenestrations in the endothelium towards Bruch's membrane; the indicated bar=2 μm;

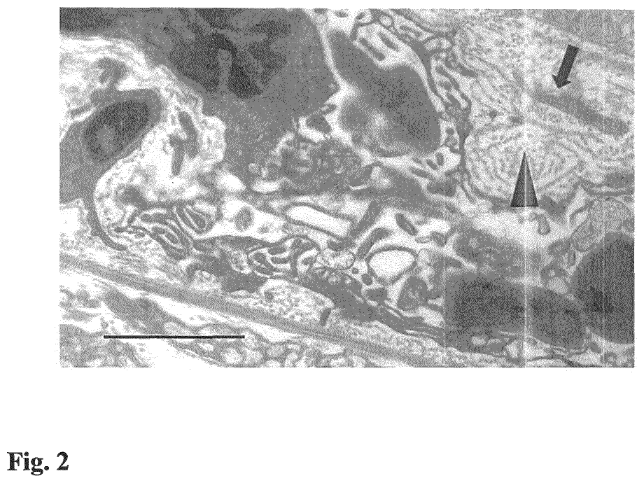

FIG. 2 is an electron micrograph taken fourteen hours after hypoxia; there were many filopodia-like projections within the capillary lumen which is largely reduced; the extracellular matrix surrounding the capillary was enhanced (arrowhead) and cells appeared within Bruch's membrane (arrow); the indicated bar=2 μm;

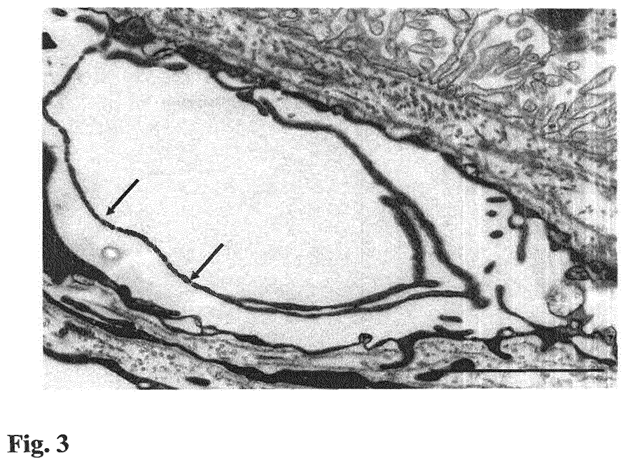

FIG. 3 is an electron micrograph taken after hypoxia; individual filipodia of the endothelium projected into the capillary lumen were more than 10 μm long (arrows); the indicated bar=2 μm;

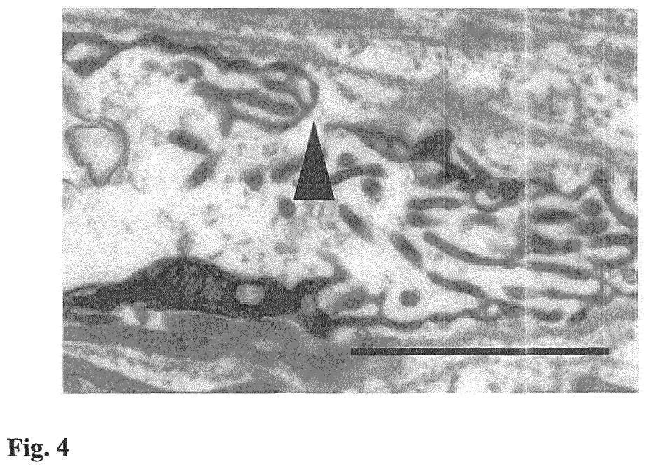

FIG. 4 is an electron micrograph taken after hypoxia; were many open gaps between or within the endothelial cells (arrowhead) of the labyrinth capillaries; the indicated bar=2 inn;

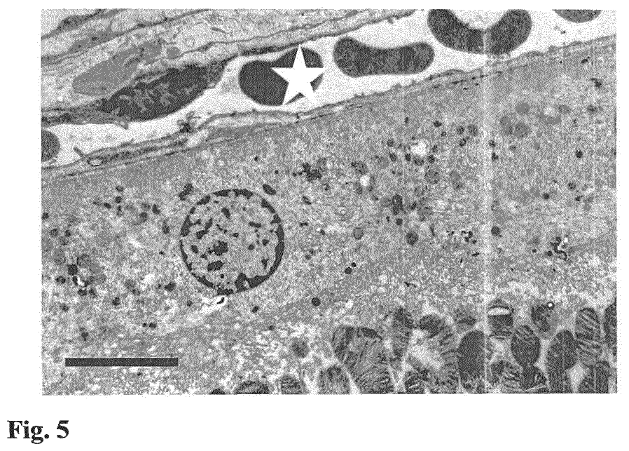

FIG. 5 is an electron micrograph taken after intravitreal PEDF injection and hypoxia; labyrinth capillaries did not develop and the lumen of the capillaries was maintained like in vivo (asterisk); the indicated bar=5 μm;

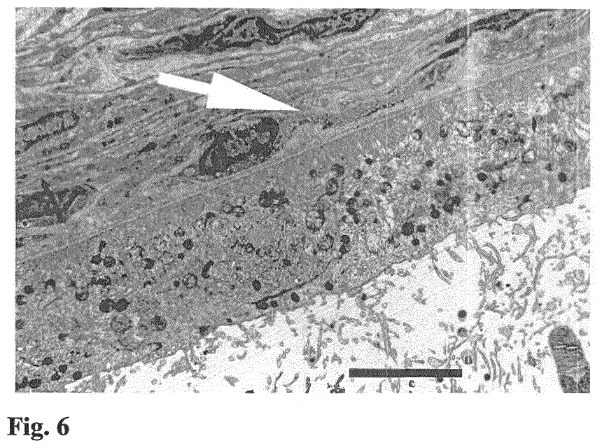

FIG. 6 is an electron micrograph taken after hypoxia and without intravitreal PEDF injection; labyrinth capillaries developed and the lumen of the capillaries was collapsed (arrow); the indicated bar=5 μm;

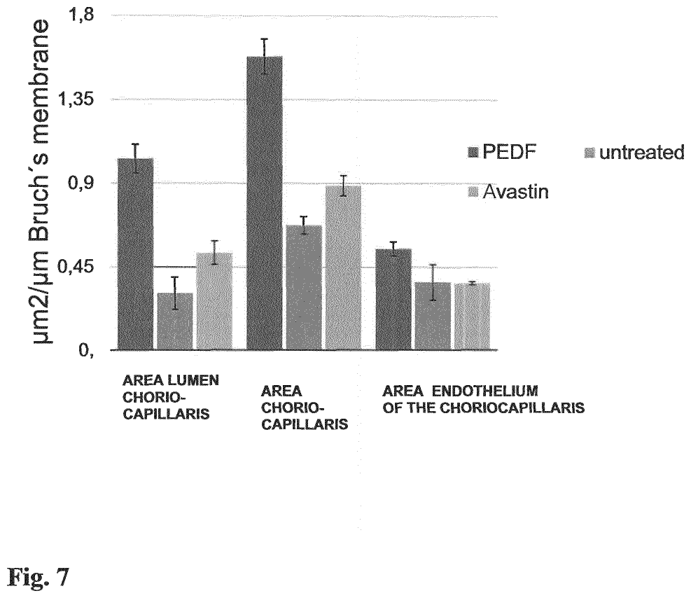

FIG. 7 is a bar diagram showing quantitative analysis of the areas occupied by the choricapillaris, by the choriocapillaris lumen and by the endothelium in ultrathin section after hypoxia and treatment with Avastin, PEDF or without treatment;

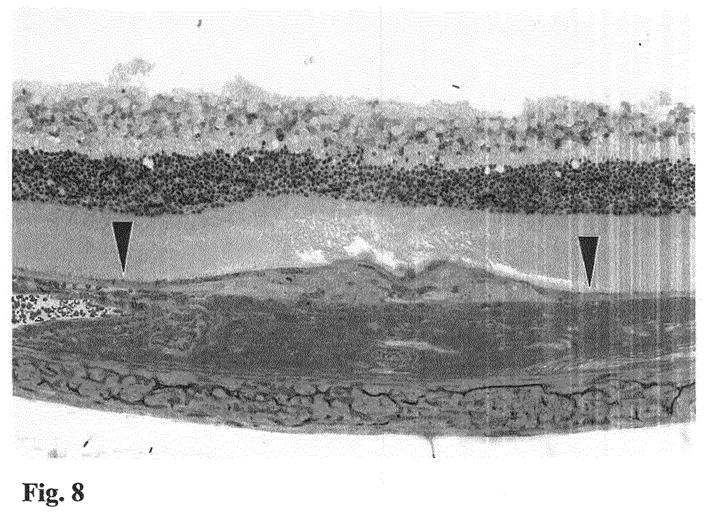

FIG. 8 is an electron micrograph of a CNV shown in a semithin section; the left and right arrow mark the extension of the CNV and the site where the RPE remains a monolayer; the photoreceptor nuclear layer is thinner and the outer segments are irregular facing the CNV;

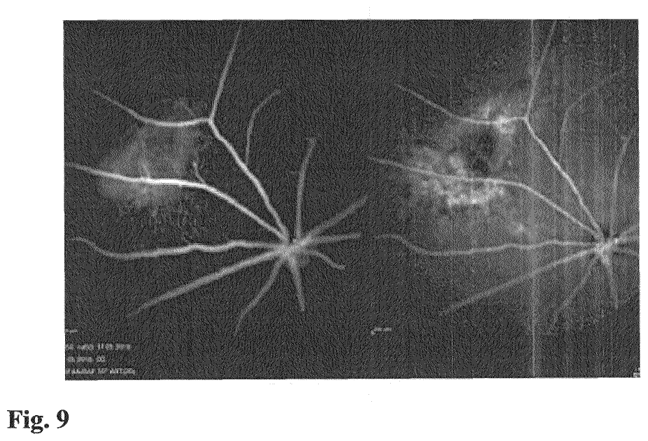

FIG. 9 shows a representative SLO angiography image about 20 min after injection of dyes (left fluorescein angiography (FA), right (indocyanine green angiography (ICG)) for an eye six weeks after VEGF-vector injection;

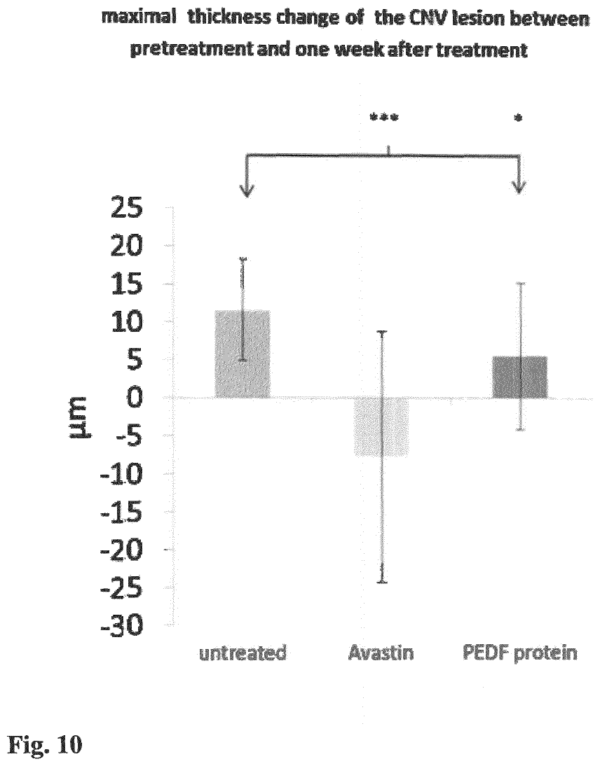

FIG. 10 is a bar diagram showing the means of change of the maximal thickness of the CNV lesion area between the measurements six (pretreatment) and seven weeks after vector injection (one week after treatment) for each group; Mean standard deviations are shown *=p<0.05, ***=p<0.0001;

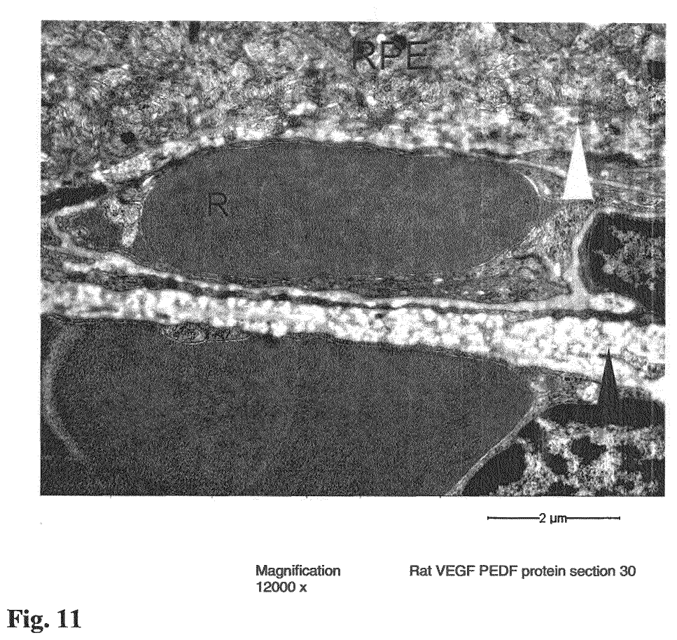

FIG. 11 is an electron micrograph showing a newly formed choriocapillaris located between Bruch's membrane (black arrowhead) and RPE; the vessel contains a red blood cell (RB); between RPE and the new vessel a new Bruch's membrane (white arrowhead) was been formed after PEDF treatment;

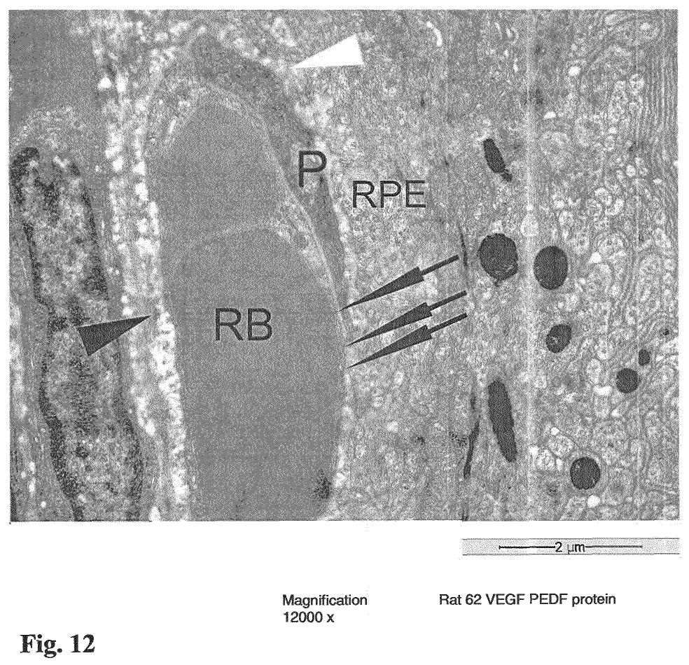

FIG. 12 is an electron micrograph showing a newly formed choriocapillaris located between Bruch's membrane (black arrowhead) and RPE; the vessel contains a red blood cell (RB); between RPE and the new vessel a new Bruch's membrane (white arrowhead) was been formed; a pericyte (P) is associated to this vessel which also is fenestrated (arrows) in the endothelium facing the RPE after PEDF treatment;

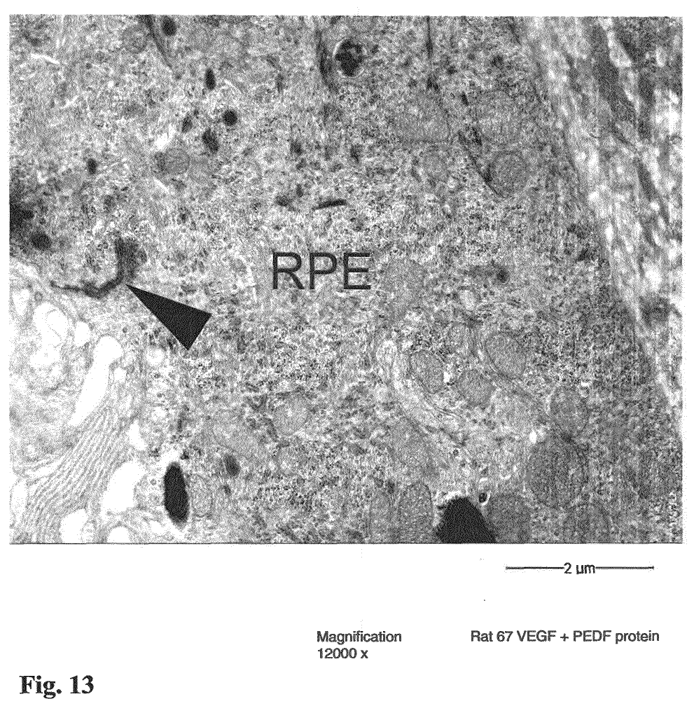

FIG. 13 is an electron micrograph showing extremely electron-dense tight junctions (arrowhead) between two RPE cells after PEDF treatment;

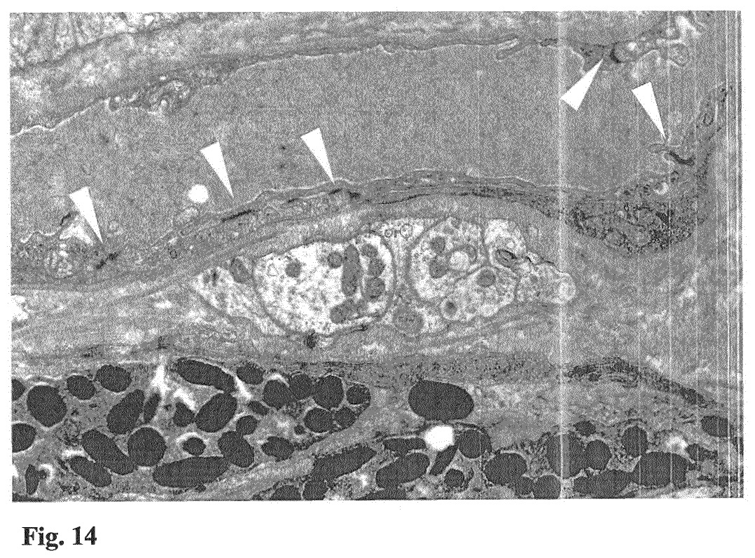

FIG. 14 is an electron micrograph showing several extremely electron dense and prominent junctions (arrowheads) between two endothelial of a choriocapillaris cells after PEDF treatment;

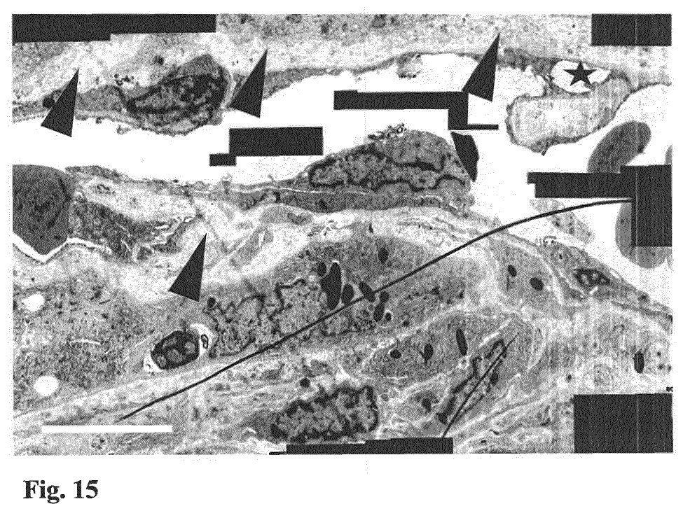

FIG. 15 is an electron micrograph shows a newly formed blood vessels which are surrounded by a thick layer of extracellular matrix (arrowheads) and separated by several layer of RPE cells from the original RPE monolayer in the absence of PEDF treatment; a protusion of extracellular matrix shifted an endothelium fold towards the vessel lumen (white asterisk); within the endothelium a large vacuole is formed (black asterisk) which is typical for pathological vessels in human CNV (Schraermeyer, Julien et al. 2015); such protrusions or vacuoles were not seen after PEDF treatment; the indicated bar=5 μm;

FIG. 16 is a panel of pictures taken by a polarizing microscope; the lower row shows sections from eyes with CNV's after picrosirius red staining; the upper row shows the same sections under polarized light; the black arrowheads mark the border between CNV and choroid. The white arrowheads indicate an immature collagen type III; the black arrow indicate the position of a ring of type I collagen surrounding a blood vessel after treatment with PEDF; the asterisks label the scleras which consist of mature collagen (type I); the left column shows an example from an eye after injection of PEDF and avastin; the middle column shows an eye that was only treated with avastin; and the right column shows an example from an eye treated with PEDF alone;

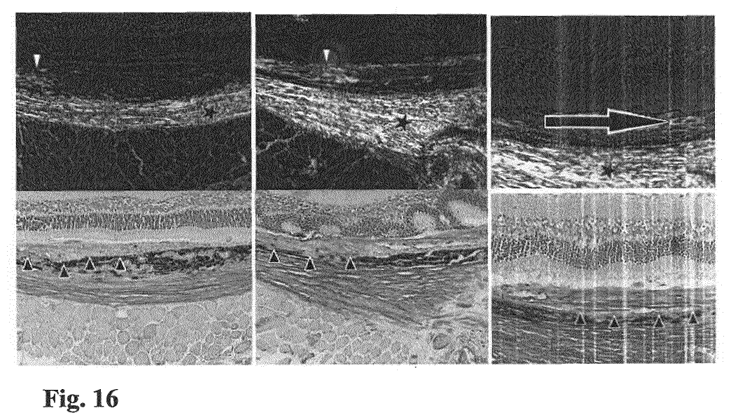

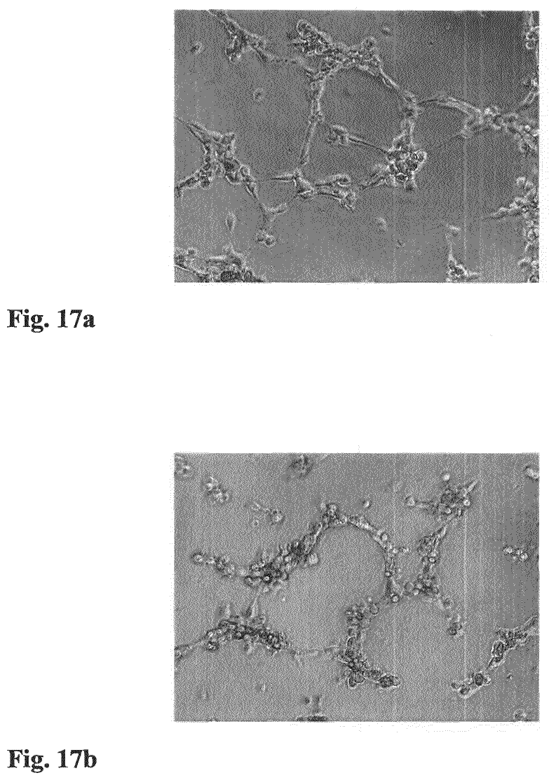

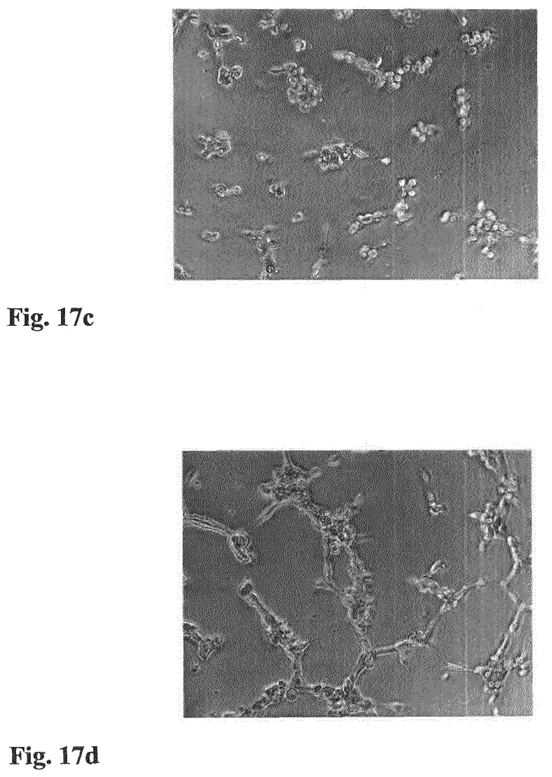

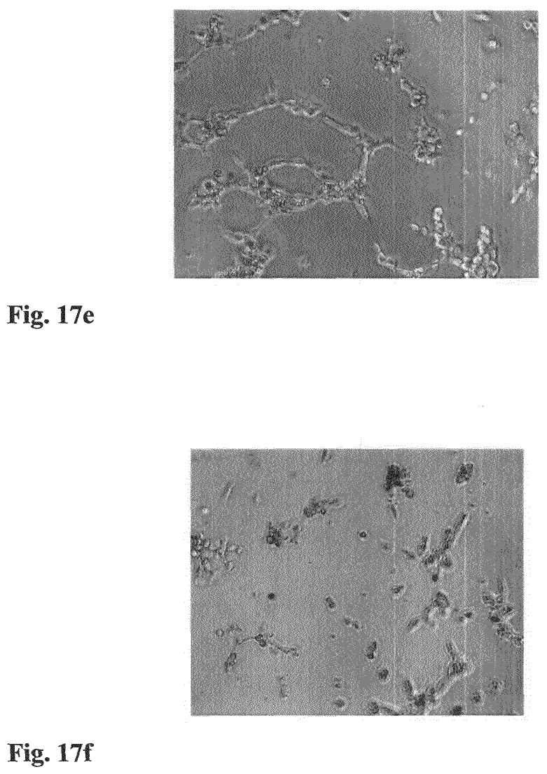

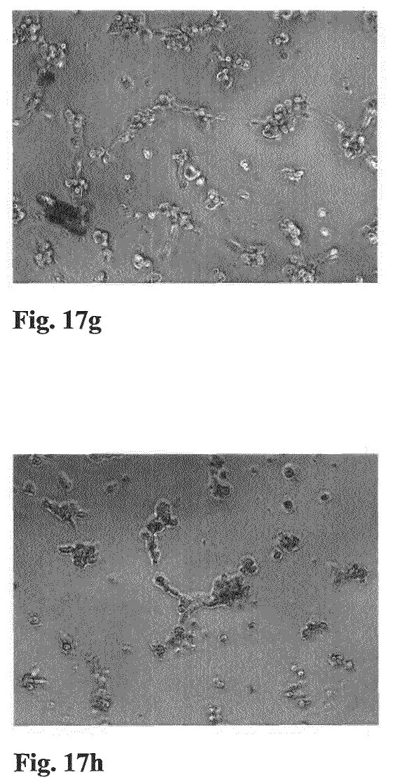

FIGS. 17a-h show microscopic photographs of endothelial cell tube formation of HUVEC on growth factor reduced Matrigel; HUVEC were left untreated (a), or treated alone with 250 ng/mL PEDF (b), 500 ng/mL PEDF (c), 250 μg/mL Bevacizumab (d) 1 mg/mL Bevacizumab (e), 2 mg/mL Bevacizumab (f), or as a combination PEDF (250 ng/mL)+Bevacizumab (250 μg/mL) (g), PEDF (250 ng/mL)+Bevacizumab (1 mg/mL) (h); Photographs were taken after 5 hours of incubation at 37° C.;

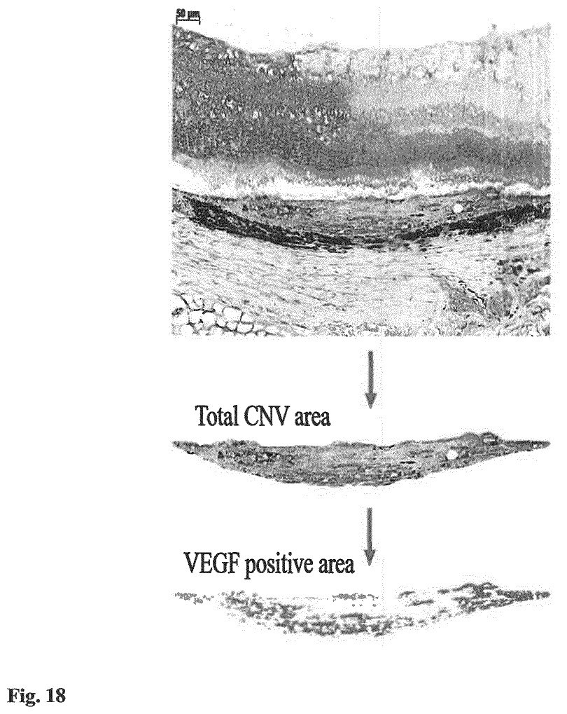

FIG. 18 is a panel of photomicrographs of a paraffin section prepared from eyes with CNVs after picrosirius red staining. The upper photomicrograph shows an overview of the section, the middle photomicrograph shows the total CNV area (“Total CNV area”) and the lower photomicrograph shows the total area of VEGF expression (“VEGF positive area”) imaged by means of an anti-VEGF antibody;

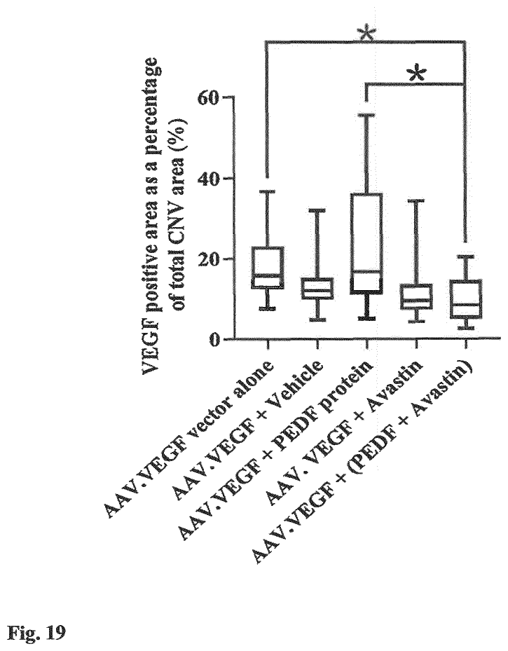

FIG. 19 is a box plot diagram indicating VEGF positive area as a percentage of total CNV are upon administration of AAV-VEGF vector alone, AAV.VEGF vector and vehicle, AAV.VEGF vector and PEDF protein, AAV.VEGF vector and Avastin, and a triple combination of AAV.VEGF vector, PEDF protein and Avastin;

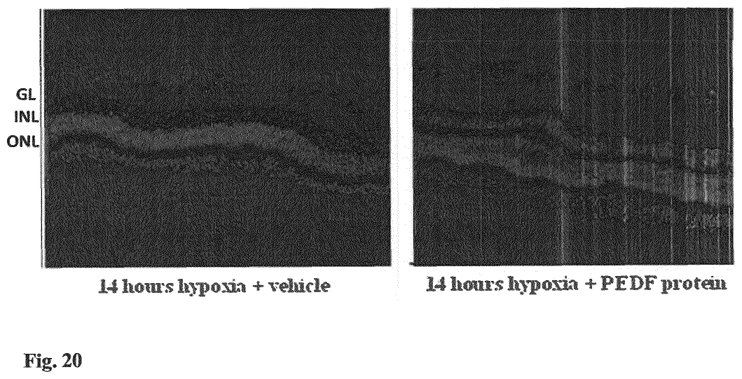

FIG. 20 is a panel of photomicrographs of a paraffin section of eyes, more specifically of the retina upon, staining. The left photomicrograph shows a retina section prepared upon 14 hours of hypoxia and vehicle injection, and the right photomicrograph shows a retina section prepared upon 14 hours of hypoxia and injection of PEDF protein;

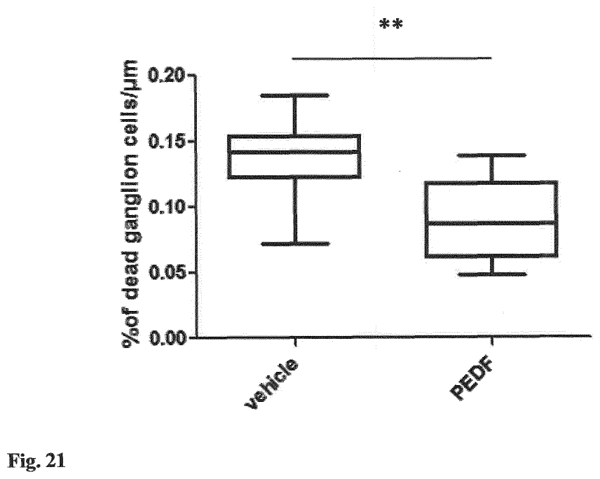

FIG. 21 is a box plot diagram illustrating the results shown in FIG. 20 and more specifically indicating the percentage (%) of dead ganglion cells/μl upon injection of vehicle or injection of PEFD protein.

FIG. 22 shows the amino acid sequence of vascular endothelial growth factor A, isoform a, of Homo sapiens (GenBank entry NP_001020537.2) (SEQ ID NO: 4);

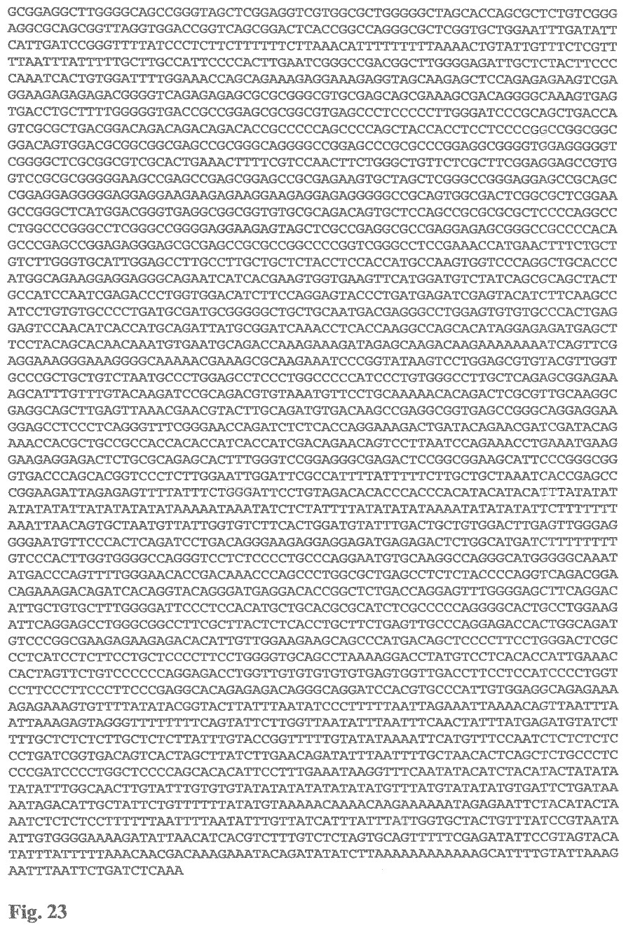

FIG. 23 shows that mRNA sequence of vascular endothelial growth factor A (VEGFA), transcript variant 1, of Homo sapiens (GenBank entry NM_001025366.3) (SEQ ID NO: 5);

FIG. 24 shows the amino acid sequence of vascular endothelial growth factor A, isoform b, of Homo sapiens (GenBank entry NP_003367.4) (SEQ ID NO: 6);

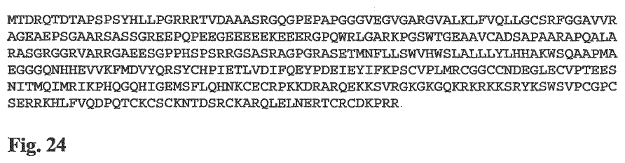

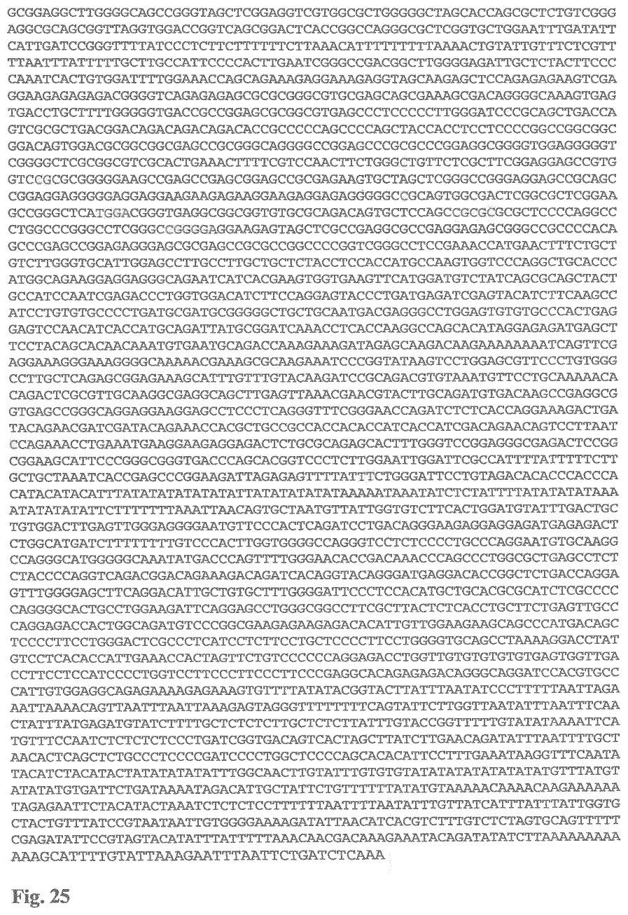

FIG. 25 shows the mRNA sequence of vascular endothelial growth factor A (VEGFA), transcript variant 2, of Homo sapiens (GenBank entry NM_003376.6) (SEQ ID NO: 7);

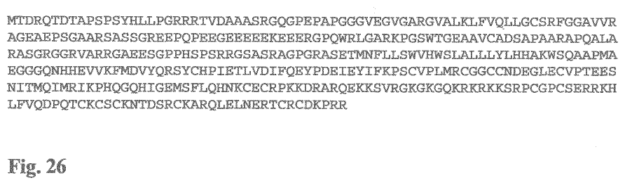

FIG. 26 shows the amino acid sequence of vascular endothelial growth factor A, isoform c, of Homo sapiens (GenBank entry NP_001020538.2) (SEQ ID NO: 8);

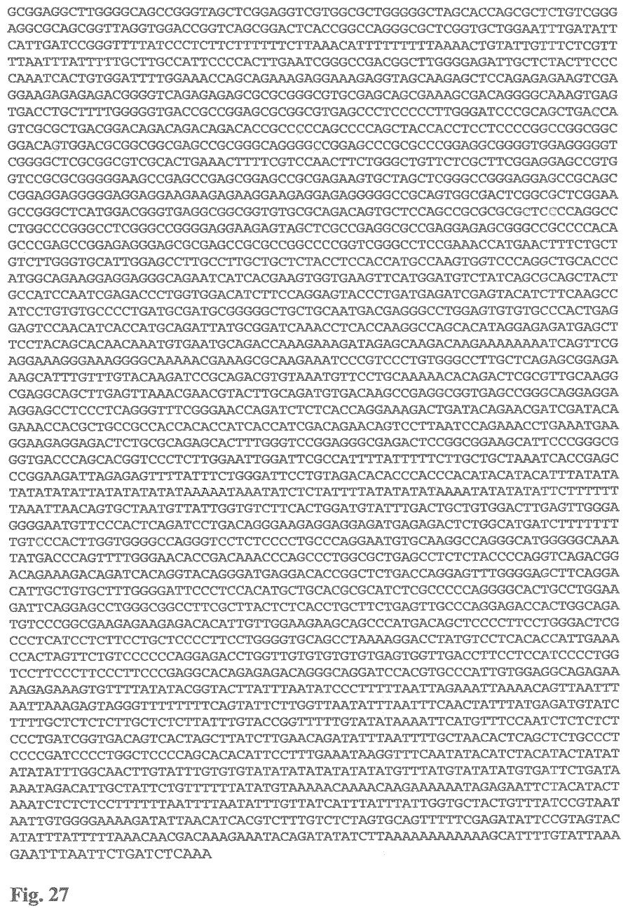

FIG. 27 shows the mRNA sequence of vascular endothelial growth factor A (VEGFA), transcript variant 3, of Homo sapiens (GenBank entry NM_001025367.3) (SEQ ID NO: 9);

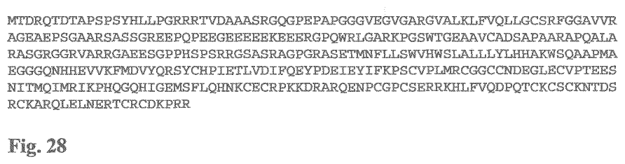

FIG. 28 shows the amino acid sequence of vascular endothelial growth factor A, isoform d, of Homo sapiens (GenBank entry NP_001020539.2) (SEQ ID NO: 10);

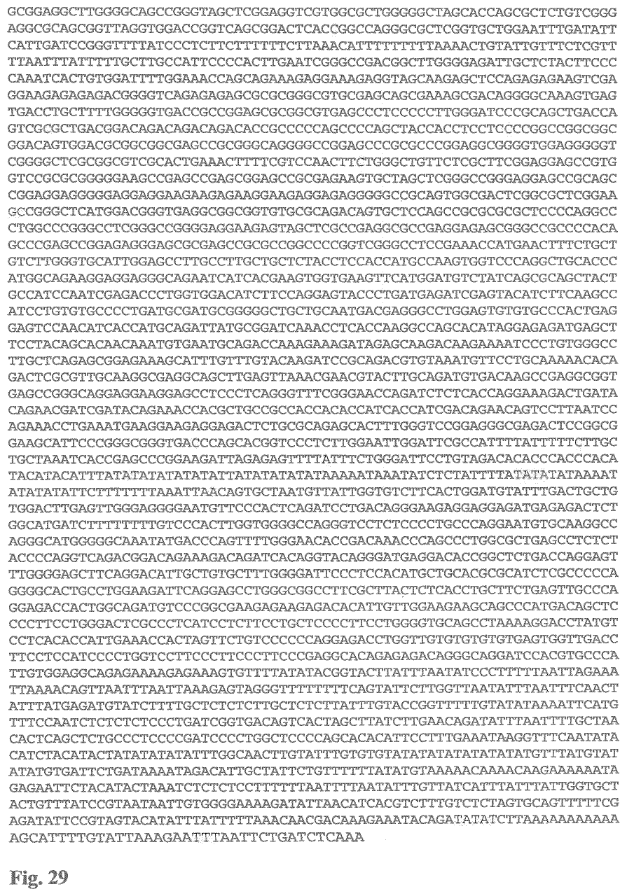

FIG. 29 shows the mRNA sequence of vascular endothelial growth factor A (VEGFA), transcript variant 4, of Homo sapiens (GenBank entry NM_001025368.3) (SEQ ID NO: 11);

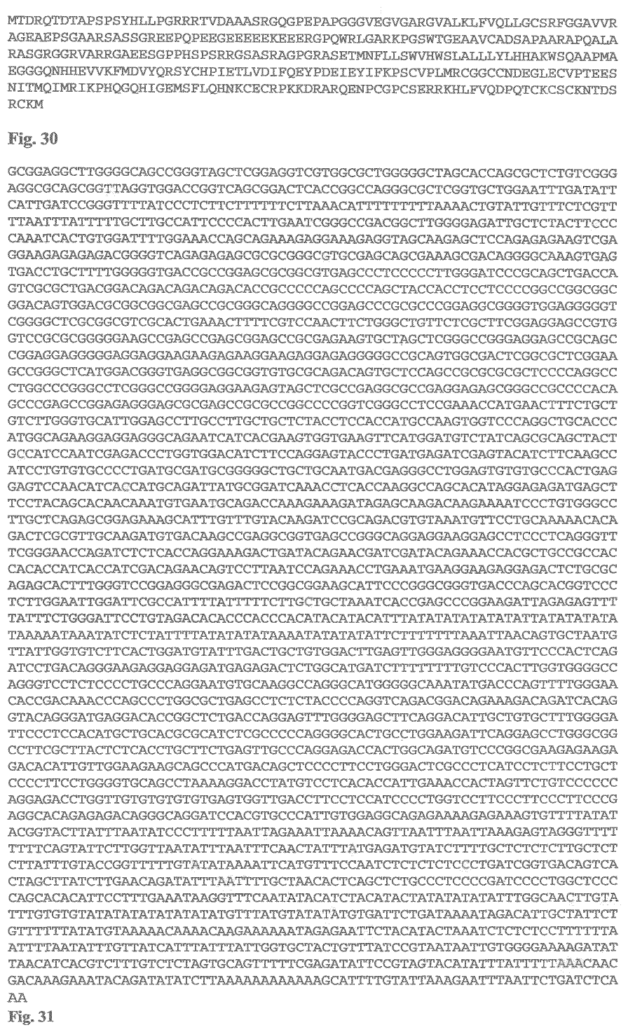

FIG. 30 shows the amino acid sequence of vascular endothelial growth factor A, isoform e, of Homo sapiens (GenBank entry NP_001020540.2) (SEQ ID NO: 12);

FIG. 31 shows the mRNA sequence of vascular endothelial growth factor A (VEGFA), transcript variant 5, of Homo sapiens (GenBank entry NM_001025369.3) (SEQ ID NO: 13);

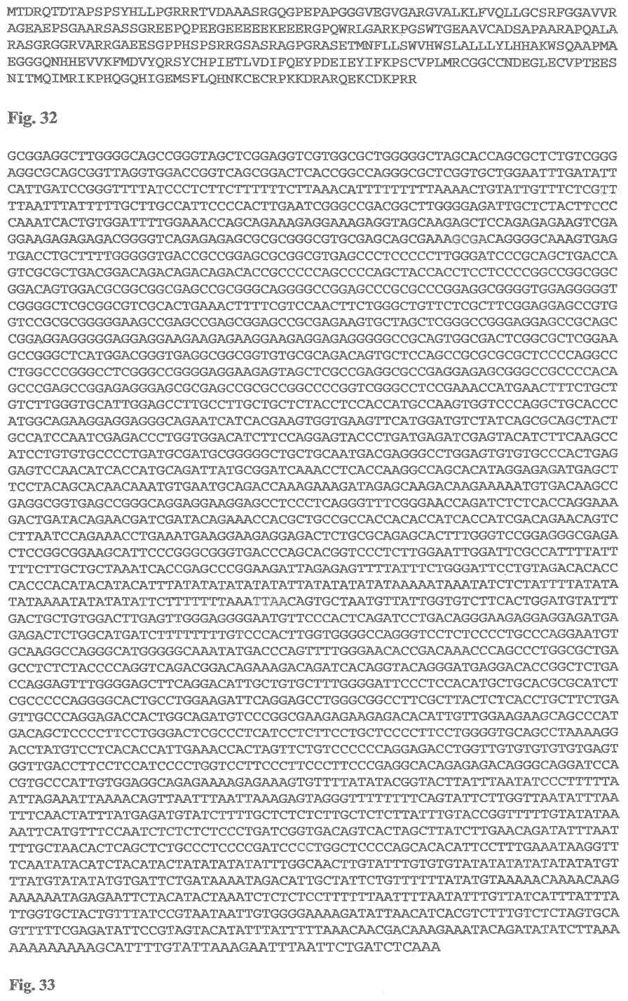

FIG. 32 shows the amino acid sequence of vascular endothelial growth factor A, isoform f, of Homo sapiens (GenBank entry NP_001020541.2) (SEQ ID NO: 14);

FIG. 33 shows the mRNA sequence of vascular endothelial growth factor A (VEGFA), transcript variant 6, of Homo sapiens (GenBank entry NM_001025370.3) (SEQ ID NO: 15);

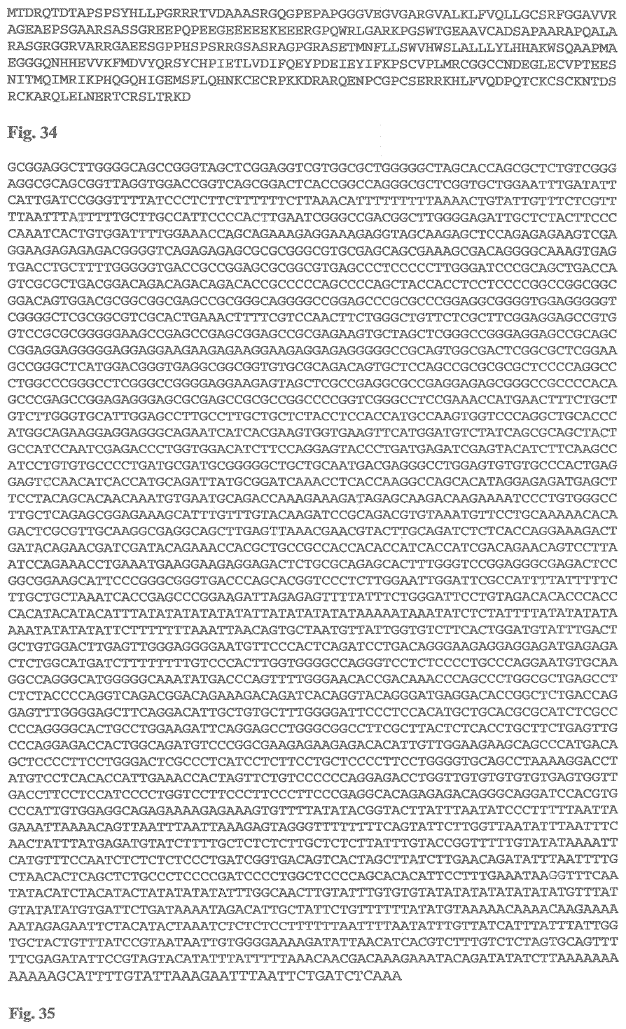

FIG. 34 shows the amino acid sequence of vascular endothelial growth factor A, isoform g, of Homo sapiens (SEQ ID NO: 16);

FIG. 35 shows the mRNA sequence of vascular endothelial growth factor A (VEGFA), transcript variant 7, of Homo sapiens (GenBank entry NM_001033756.3) (SEQ ID NO: 17);

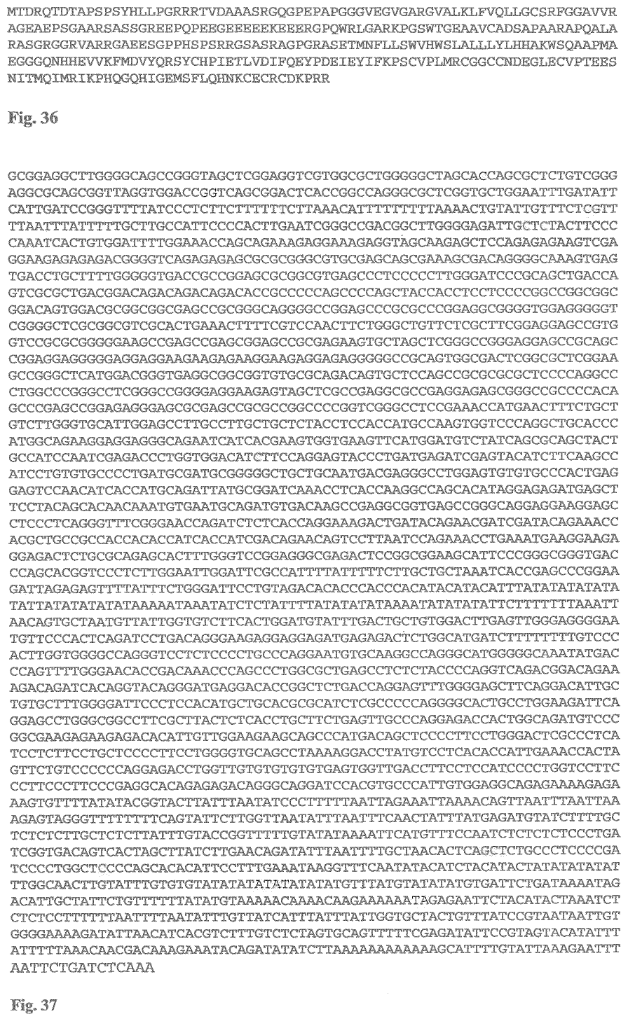

FIG. 36 shows the amino acid sequence of vascular endothelial growth factor A, isoform h, of Homo sapiens (GenBank entry NP_001165093.1) (SEQ ID NO: 18);

FIG. 37 shows that mRNA sequence of vascular endothelial growth factor A (VEGFA), transcript variant 8, of Homo sapiens (GenBank entry NM_001171622.) (SEQ ID NO: 19);

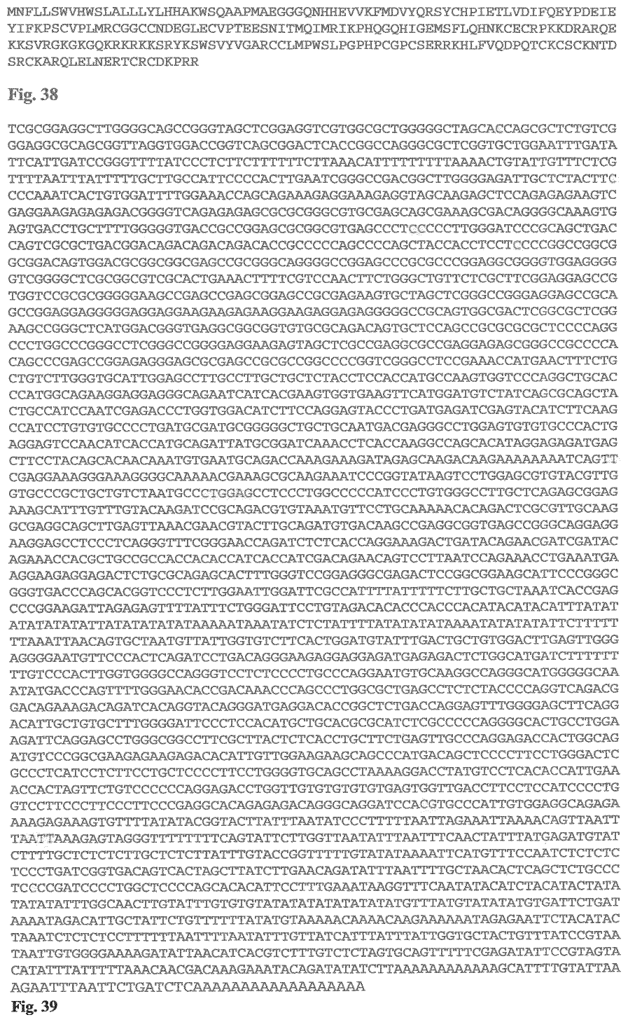

FIG. 38 shows that amino acid sequence of vascular endothelial growth factor A, isoform i precursor, of Homo sapiens (GenBank entry NP_001165094.1) (SEQ ID NO: 20);

FIG. 39 shows that mRNA sequence of vascular endothelial growth factor A (VEGFA), transcript variant 1, of Homo sapiens (GenBank entry NM_001171623.1) (SEQ ID NO: 21);

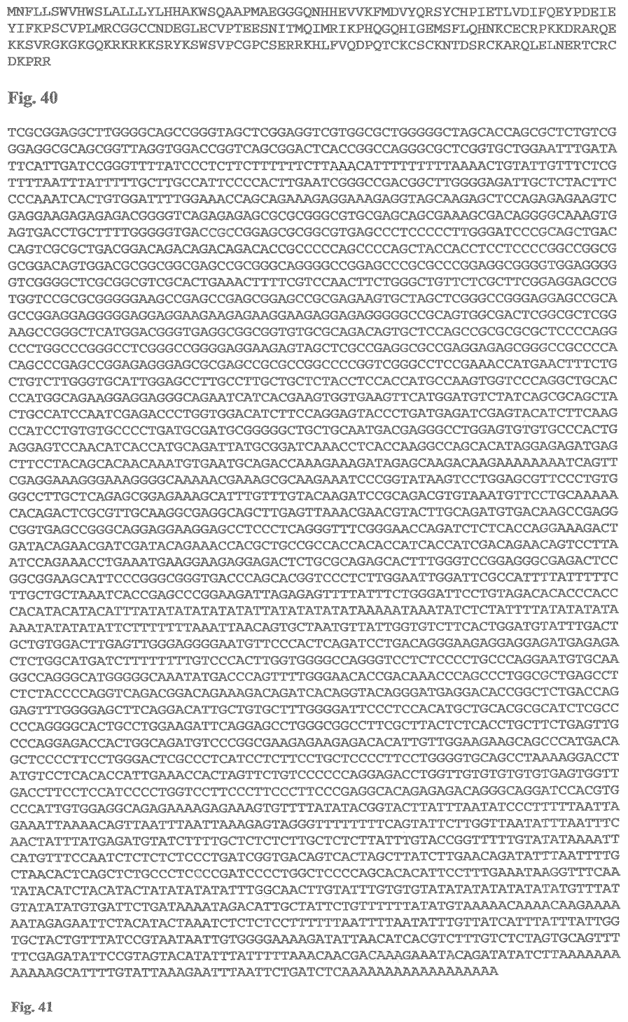

FIG. 40 shows the amino acid sequence of vascular endothelial growth factor A, isoform j precursor, of Homo sapiens (GenBank entry NP_001165095.1) (SEQ ID NO: 22);

FIG. 41 shows the mRNA sequence of vascular endothelial growth factor A (VEGFA), transcript variant 2, of Homo sapiens (GenBank entry NM_001171624.1) (SEQ ID NO: 23);

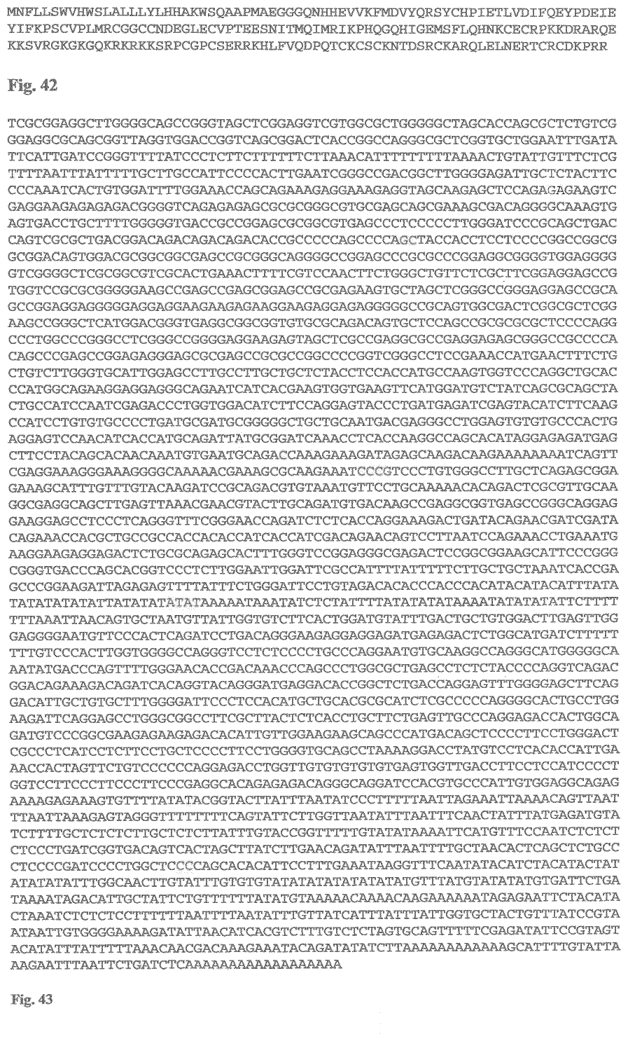

FIG. 42 shows the amino acid sequence of vascular endothelial growth factor A, isoform k precursor, of Homo sapiens (GenBank entry NP_001165096.1) (SEQ ID NO: 24);

FIG. 43 shows the mRNA sequence of vascular endothelial growth factor A (VEGFA), transcript variant 3, of Homo sapiens (GenBank entry NM_001171625.1) (SEQ ID NO: 25);

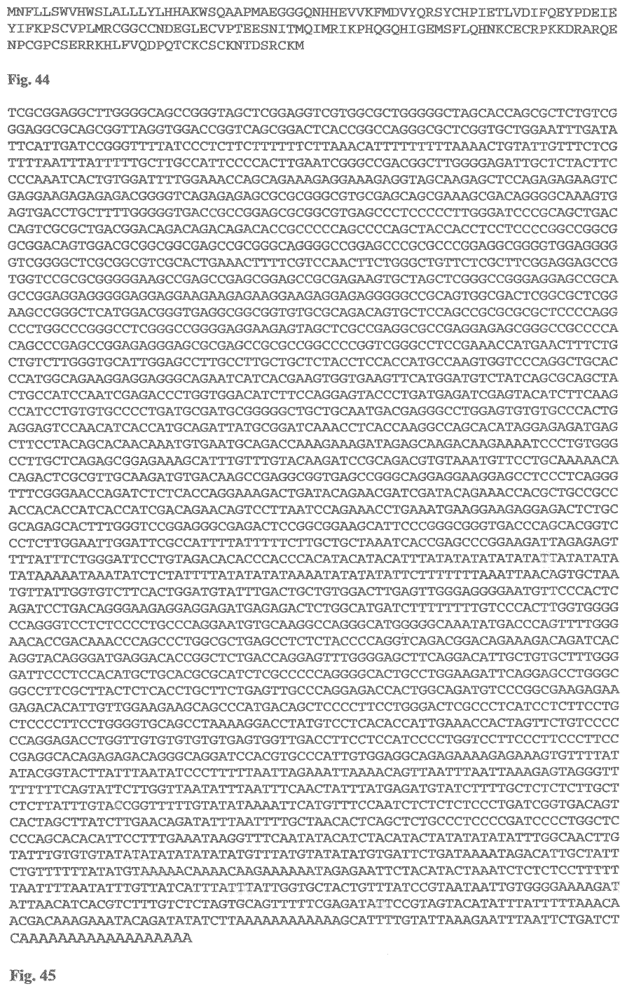

FIG. 44 shows the amino acid sequence of vascular endothelial growth factor A, isoform m precursor, of Homo sapiens (GenBank entry NP_001165098.1) (SEQ ID NO: 26);

FIG. 45 shows the mRNA sequence of vascular endothelial growth factor A (VEGFA), transcript variant 5, of Homo sapiens (GenBank entry NM_001171627.1) (SEQ ID NO: 27);

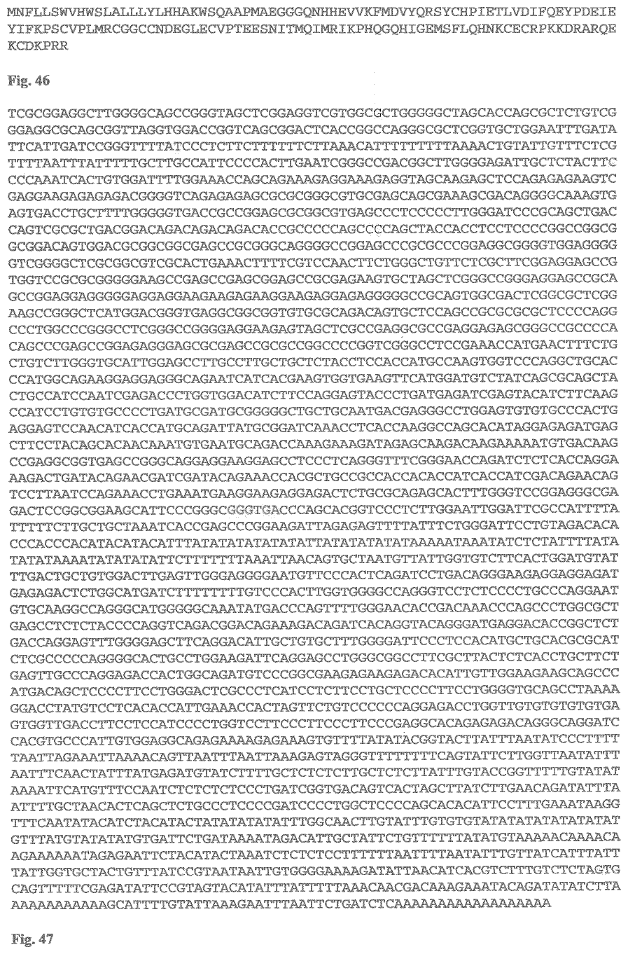

FIG. 46 shows the amino acid sequence of vascular endothelial growth factor A isoform n precursor, of Homo sapiens (GenBank entry NP_001165099.1) (SEQ ID NO: 28);

FIG. 47 shows the mRNA sequence of vascular endothelial growth factor A (VEGFA), transcript variant 6, of Homo sapiens (GenBank entry NM_001171628.1) (SEQ ID NO: 29);

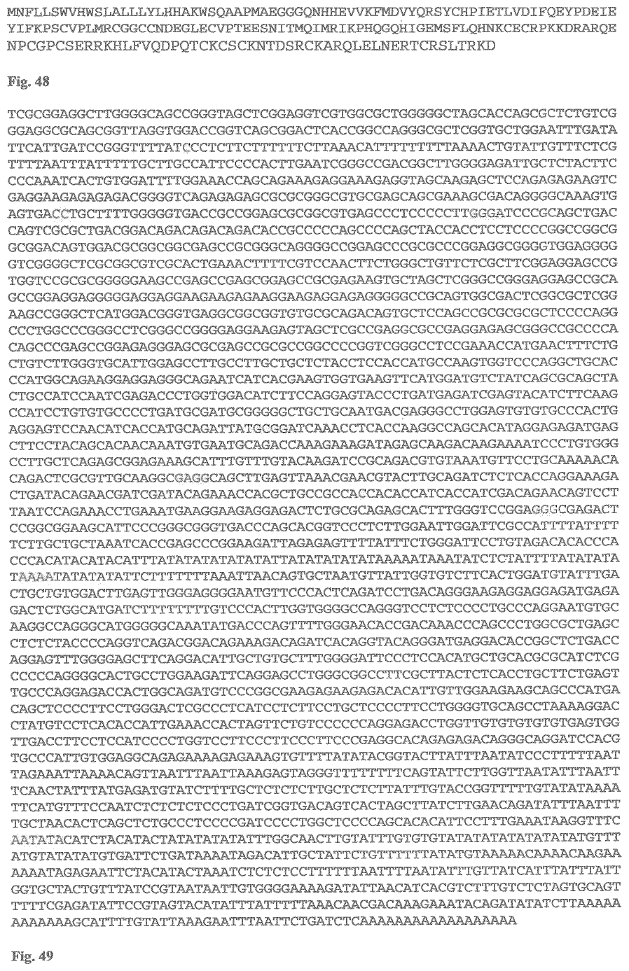

FIG. 48 shows the amino acid sequence of vascular endothelial growth factor A, isoform o precursor, of Homo sapiens (GenBank entry NP_001165100.1) (SEQ ID NO: 30);

FIG. 49 shows that mRNA sequence of vascular endothelial growth factor A (VEGFA), transcript variant 7, of Homo sapiens (GenBank entry NM_001171629.1) (SEQ ID NO: 31);

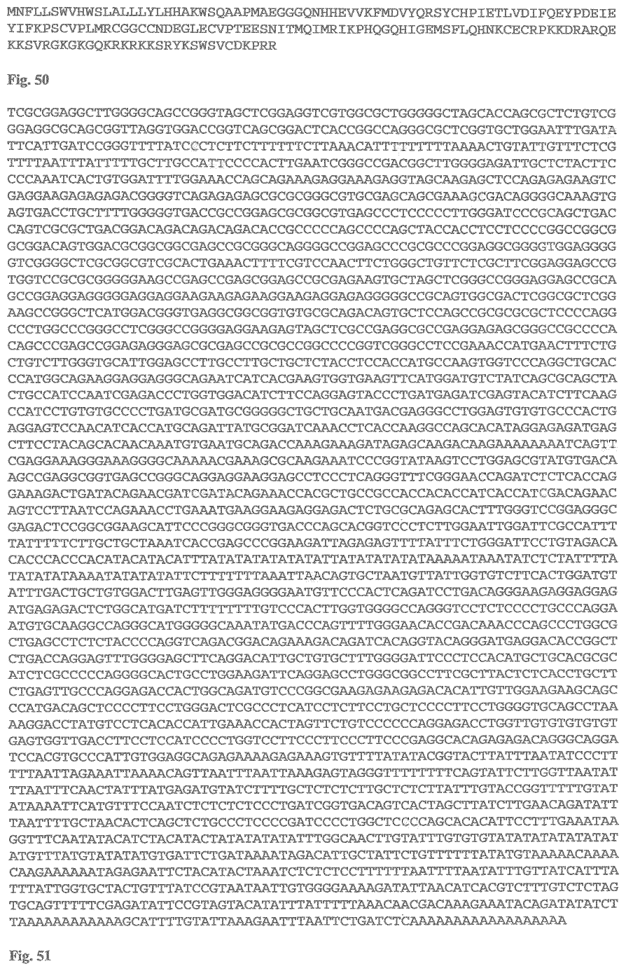

FIG. 50 shows the amino acid sequence of vascular endothelial growth factor A, isoform q precursor, of Homo sapiens (GenBank entry NP_001191313.1) (SEQ ID NO: 32);

FIG. 51 shows that mRNA sequence of vascular endothelial growth factor A (VEGFA), transcript variant 9, of Homo sapiens (GenBank entry NM_001204384.1) (SEQ ID NO: 33);

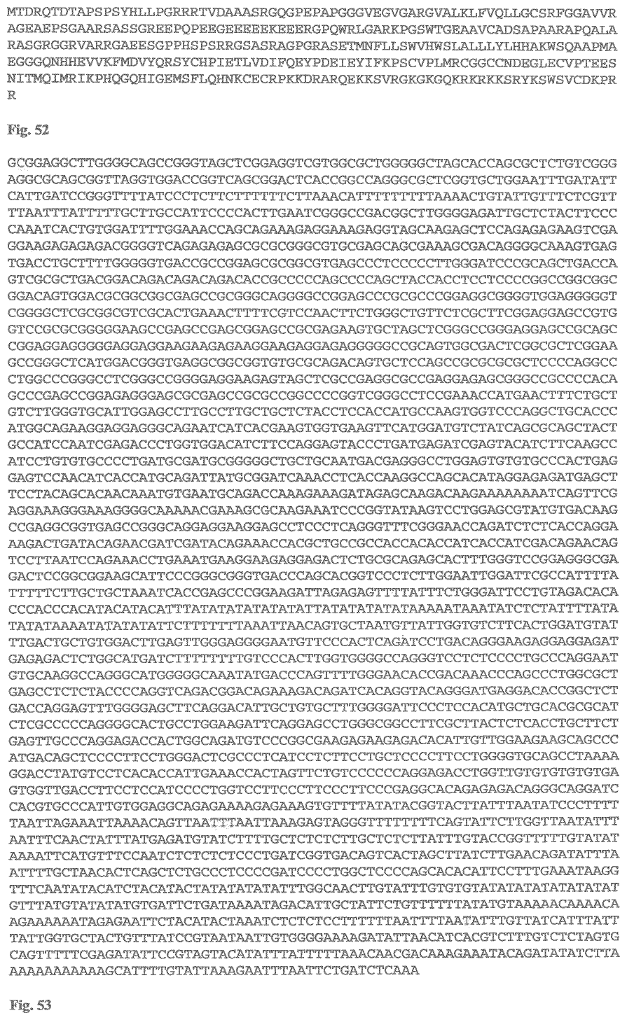

FIG. 52 shows that amino acid sequence of vascular endothelial growth factor A, isoform r, of Homo sapiens (GenBank entry NP_001191314.1) (SEQ ID NO: 34);

FIG. 53 shows the mRNA sequence of vascular endothelial growth factor A (VEGFA), transcript variant 9, of Homo sapiens (GenBank entry NM_001204385.2) (SEQ ID NO: 35);

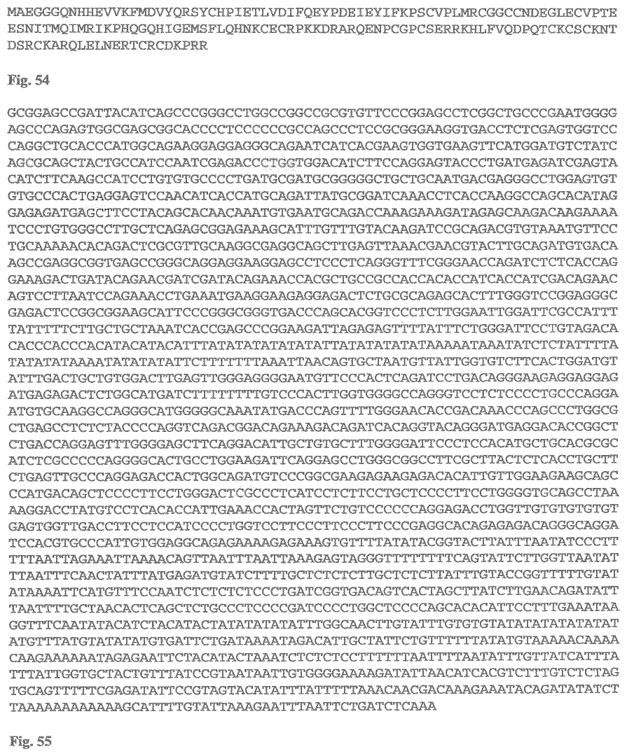

FIG. 54 shows that amino acid sequence of vascular endothelial growth factor A, isoform s, of Homo sapiens (GenBank entry NP_001273973.1) (SEQ ID NO: 36);

FIG. 55 shows the mRNA sequence of vascular endothelial growth factor A (VEGFA), transcript variant 10, of Homo sapiens (GenBank entry NM_001287044.2) (SEQ ID NO: 37);

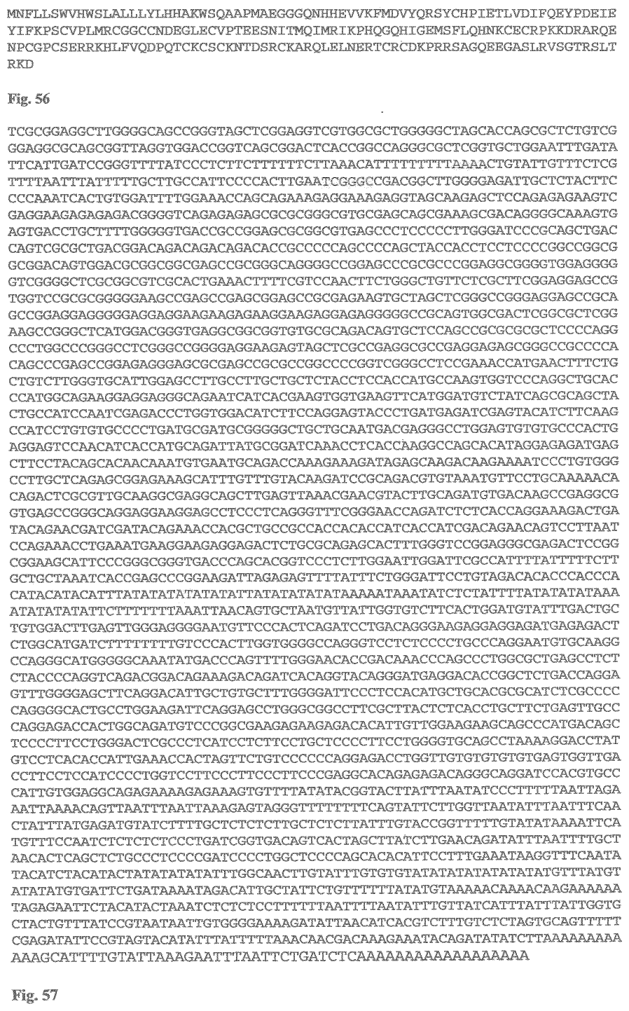

FIG. 56 shows the amino acid sequence of vascular endothelial growth factor A, isoform VEGF-Ax precursor, of Homo sapiens (GenBank entry NP_001303939.1) (SEQ ID NO: 38);

FIG. 57 shows the mRNA sequence of vascular endothelial growth factor A (VEGFA), transcript variant 4, of Homo sapiens (GenBank entry NM_001317010.1) (SEQ ID NO: 39);

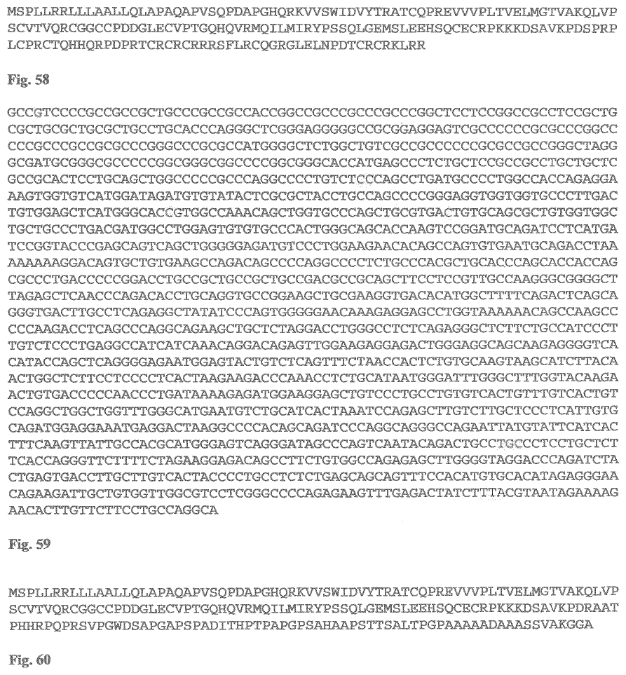

FIG. 58 shows the amino acid sequence of vascular endothelial growth factor B, isoform VEGFB-167 precursor, of Homo sapiens (GenBank entry NP_001230662.1) (SEQ ID NO: 40);

FIG. 59 shows that mRNA sequence of vascular endothelial growth factor B (VEGFB), transcript variant VEGFB-167, of Homo sapiens (GenBank entry NM_001243733.2) (SEQ ID NO: 41);

FIG. 60 shows that amino acid sequence of vascular endothelial growth factor B, isoform VEGFB-186 precursor, of Homo sapiens (GenBank entry NP_003368.1) (SEQ ID NO: 42);

FIG. 61 shows that mRNA sequence of vascular endothelial growth factor B (VEGFB), transcript variant VEGFB-186, of Homo sapiens (GenBank NM_003377.5) (SEQ ID NO: 43);

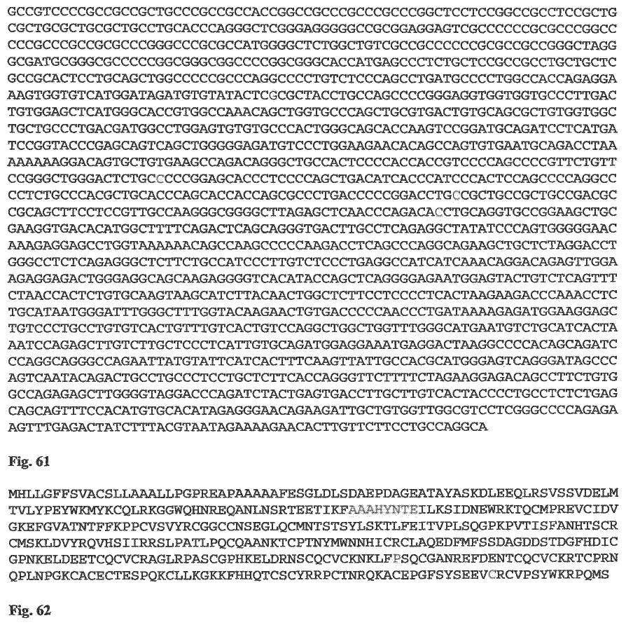

FIG. 62 shows the amino acid sequence of vascular endothelial growth factor C preproprotein of Homo sapiens (GenBank entry NP_005420.1) (SEQ ID NO: 44);

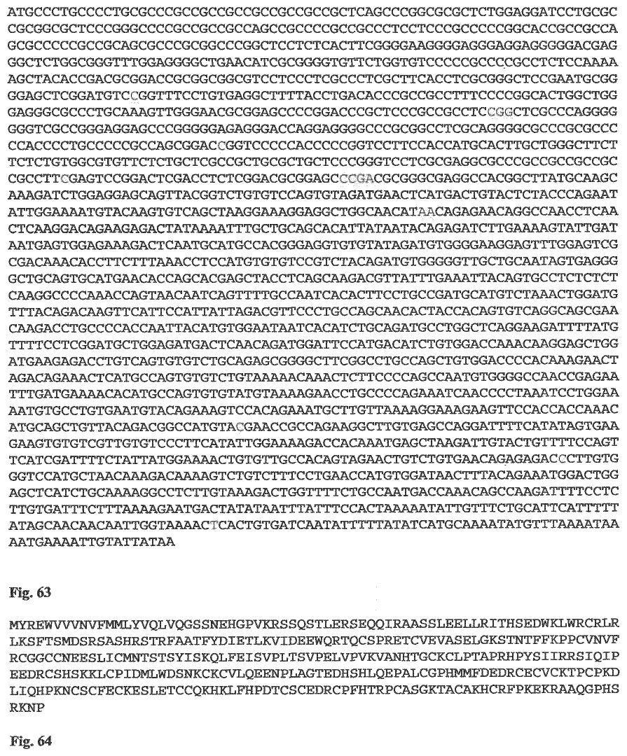

FIG. 63 shows the mRNA sequence of vascular endothelial growth factor C (VEGFC) of Homo sapiens (GenBank entry NM_005429.5) (SEQ ID NO: 45);

FIG. 64 shows the amino acid sequence of vascular endothelial growth factor D preproprotein of Homo sapiens (GenBank entry NP_004460.1) (SEQ ID NO: 46); and

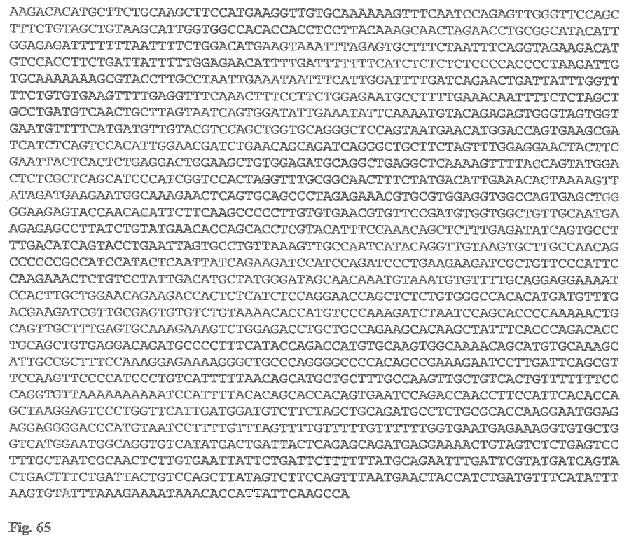

FIG. 65 shows the mRNA sequence of vascular endothelial growth factor D (VEGFD) of Homo sapiens (GenBank entry NM_004469.5) (SEQ ID NO: 47).

### EXAMPLE 1: EXPOSURE OF EYES TO HYPOXIA

Thirty-two eyes from 16 rats were exposed to mild hypoxia. Ischemia was modeled by incubating the eyes in DMEM at 4° C. during fourteen hours after enucleation in 15 ml Falcon tubes (1 eye per tube). Hypothermia can prolong the tolerance time to an ischemic insult. The tubes were filled with 7 ml DMEM and air. They were stored horizontally to enhance the oxygen exchange between DMEM and air. After 14 hours half of the eyes were embedded into paraffin for immunocytochemistry or into Epon for electron microscopy. Twelve eyes were embedded directly after enucleation and served as controls.

The oxygen pressure was measured by a calibrated fiber optic oxygen sensor (WPI, Friedberg, Germany) which was inserted into the vitreous body of eyes in this ex vivo experiment and for comparison in eyes of living rats under anesthesia. Directly after enucleation the oxygen pressure dropped down to 2% of the in vivo concentration and then gradually increased and reached the in vivo concentration after 1 hour. After that the in vivo oxygen concentration was not undercut.

### EXAMPLE 2: MEASUREMENT OF THE INNER CIRCUMFERENTIAL CONTOUR OF THE FILIPODIA-LIKE PROJECTIONS OF THE ENDOTHELIAL CELLS

Electron micrographs from choriocapillaris vessels from each plastic embedded eye were analyzed for the inner circumferential contour of the filopodia-like projections. Also, the length of the outer endothelial cell circumferential contour per sectioned vessel area was measured. The iTEM image analysis software (iTEM version 5.0; Olympus Soft Imaging Solutions, Munster, Germany) was used for the measurements. The results were analyzed in Microsoft Excel 2011 and IBM SPSS Statistics 22 software by using a nonparametric Mann-Whitney test. A p-value of less than 0.05 was considered significantly different between groups. The length of the inner endothelial cell circumferential contour increased by 58% (p<0.001) in comparison to the control group which indicates the formation of microvillar endothelial cell projections towards the vessel lumen. These vessels correspond exactly to the labyrinth capillaries in human CNV (Schraermeyer, Julien et al. 2015).

### EXAMPLE 3: EFFECT OF HYPOXIA ON CHORIOCAPILLARIS

The choriocapillaris without exposure to hypoxia contains a regular thin endothelium with fenetrations towards the side of Bruch's membrane (see, FIG. 1 arrows). The lumen of the capillaries is lacking any cellular projections. Fourteen hours after hypoxia there were many filopodia-like projections within the capillary lumen. (see, FIG. 2). The extracellular matrix surrounding the capillary was enhanced (arrowhead) and cells appeared within Bruch's membrane (arrow). Individual filipodia within the capillary lumen were more than 10 μm long (see, FIG. 3 arrows). After hypoxia there were many open gaps between or within the endothelial cells (see, FIG. 4 arrowhead).

### EXAMPLE 4: EXPRESSION OF VEGF AND HIF-1α AFTER HYPOXIA

Control and ischemic eyes were formalin-fixed and paraffin-embedded according to standard procedures. 4-μm thick sections were cut, deparaffinized and rehydrated, and boiled in a citrate buffer (pH=6.0). After three washing steps in TBS (pH=7.6), immuno-histochemical staining of HIF-1α and VEGF were performed according to the instructions provided by the manufacturer in a humid chamber. The slides were incubated for 120 min with the primary rabbit anti-HIF-la antibody (1:100, Abcam, Denmark) at 37° C. and then processed using the DAKO REAL Detection System Alkaline Phosphatase/RED kit rabbit/mouse, then counterstained with hematoxylin and covered. The same procedure was performed for immune reactivity analysis against VEGF using a primary mouse antibody (1:50, Gene Tex, USA) and boiling in TBS buffer (pH=9.0).

In control rats, HIF-la was not expressed in the choroid. After Hypoxia HIF-la was detected in the choroid. VEGF in the control eyes was detected within the RPE. After 14 hours of ischemia the staining of VEGF appeared additionally in the retina and the choroid.

### EXAMPLE 5: INHIBITION OF LABYRINTH CAPILLARY FORMATION BY PEDF

12 eyes were injected with 20 μg PEDF (BioVendor) and then exposed to hypoxia as described in example 1. Three eyes were injected with 0.8 μl Bevacizumab (Avastin). Six eyes were also exposed to hypoxia without any injection. Ultrathin sections of the eyes were investigated under the electron microscope.

Without treatment the choriocapillaris changed into labyrinth capillaries with gaps between the endothelium as shown in FIGS. 1 to 4 and collapsed leading to often complete loss of the capillary lumen (see, arrow in FIG. 5). In contrast the lumen of the choriocapillaris appeared like after in-vivo fixation and were well preserved (see, asterisk in FIG. 6).

The areas enclosed by the inner and outer endothelial cell circumferential contour per sectioned vessel were measured in electron micrographs from all eyes. From these measurements the areas occupied by the entire choriocapillaris, by the lumen of the choriocapillaris and by the endothelium were calculated. PEDF not only inhibited the formation of endothelial filopodia towards the vessel lumen and gaps, it also preserved the vessel lumina significantly better than without treatment (see, FIG. 7) (p<0.0000003) and compared to Avastin treatment (p<0.023). Also, the area of the sectioned endothelial cells, which is likely proportional to the volume of the cells, was significantly larger compared to untreated (see, FIG. 7 right) (p<0.03) whereas Avastin had no effect (p=0.75).

Students t-test was performed to compare the results of the different experimental groups. For the analysis the excel software was used. The error probability was 5% (p<0.05 statistically significant).

### EXAMPLE 6: FORMATION OF FUNCTIONAL TIGHT CHORIOCAPILLARIS AND BRUCH'S MEMBRANE AFTER VEGF OVEREXPRESSION AND PEDF TREATMENT

A new vector system was designed for this project, using the same VEGF cassette as in the adeno-vector studies before (Julien, Kreppel et al. 2008). Human VEGF-A165 cDNA, from the plasmid pBLAST49-hVEGF (Invivogen, San Diego, CA) was inserted in a state of the art AAV2 vector (subtype 4) backbone produced by Sirion Biotech GmbH (Munich, Germany). The new AAV vector has the benefit that it contains an RPE specific RPE65 promotor instead of the unspecific CMV promotor used before in adenoviral studies. These new AAV-vectors (e.g. AAV-VEGF) are less toxic and have a slower expression rate with longer expression time as compared to the adeno-vectors, favorable for long time expression studies dedicated for evaluation of drug candidates for treatment over a time frame of several months (Rolling, Le Meur et al. 2006).

Subretinal injection of AAV.VEGF-A165 vector in rat eyes 2×10⁹ virus particles of the AAV-VEGF vector, diluted in 2 μl PBS were sub-retinally injected in both eyes of 30 Long Evans rats. Briefly, after anaesthesia with an intraperitoneal injection of a three component narcosis (0.005 mg fentanyl, 2 mg midazolam and 0.15 mg of medetomidine/kg body weight), the pupils were dilated with 1 to 2 drops of Medriaticum drops (Pharmacy of the University of Tubingen, Germany) and a drop of topical anaesthetic Novesine (OmniVision, Puchheim, Germany) was applied. Methocel (OmniVision, Puchheim, Germany) eye drops were used to avoid drying of the eyes. Injections were performed using a surgical microscope. The sclera was first opened with a 25 G needle close to the limbus, then 2 μl of vector suspension (2 μl contain 2×10⁹ virus particles AAV-VEGF, max. possible dose) were injected sub-retinally (pars plana) using a 10 μl NanoFil syringe with a NanoFil 34 G blunt needle (World Precision Instruments). Topical antibiotic eye drops Gentamicin-POS® (Ursapharm, Saarbrücken, Germany) were applied after the injection. The anaesthesia was neutralized by subcutaneous injection of an antidote (0.12 mg naloxon, 0.2 mg flumazenil, 0.75 mg atipamezol/kg body weight).

Intravitreal Injection

Intravitreal injection of the therapeutic substances was made 6 weeks after VEGF vector injection

For the intravitreal injections, a small incision was made into the conjunctiva at the outer corner of the eyes. The eyeball was rotated by grasping the conjunctiva with a pair of fine tweezers and gentle pulling. A volume of 5 μl was injected through the hole intravitreally using a 10 μl NanoFil syringe with a NanoFil 34 gauge bevelled needle (World Precision Instruments). After the injection, the needle remained in the eye for an additional 3 or 4 seconds to reduce reflux and was then drawn back. The eyeball was brought back into its normal position, and the antibiotic ointment was applied to the eye. The whole procedure was performed using a surgical microscope equipped with illumination. Three groups were investigated.

1) Avastin® (bevacizumab; 25 mg/ml; Roche) was injected intravitreally into 20 eyes: It was purchased and aliquoted by the Pharmacy of the University Hospital of Tubingen. 100 mg of Avastin® were diluted in four milliliters of the vehicle solution contains 240 mg α,α-trehalose 2 H2O, 23.2 mg Na2HPO4 H2O, 4.8 mg NaH2PO4, and 1.6 mg polysorbate 20.

2) PEDF human HEK293 recombinant protein (1 μg/μl; BioVendor) was injected intravitreally into 20 eyes.

The pellet of the of the recombinant protein was filtered (0.4 μm) and lyophilized in 0.5 mg/mL in 20 mM TRIS, 50 mM NaCl, pH 7.5. According to the product data sheet, it was dissolved in deionized water (Ampuwa water) in order to obtain a working stock solution of 1 μg/μl.

3) 20 eyes were not treated

In vivo imaging (SLO/OCT, FA and ICG angiographies) and quantifications were performed according to (Wang, Rendahl et al. 2003). Subretinal AAV-VEGF leads to RPE proliferation 5 weeks to 20 months after injections, leakage can be observed starting from 2-12 months by fluorescein angiography. Therefore, scanning laser ophthalmoscopy (SLO), optical coherence tomography (OCT), fluorescein angiography (FA) and indocyanine green angiography (ICG) were performed 6 weeks after vector injection. The eyes were reinvestigated 7 weeks after injection of the VEGF vector using a Spectralis™ HRA+OCT (Heidelberg Engineering, Heidelberg, Germany) device modified for the use with animals according to protocols from (Fischer, Huber et al. 2009, Huber, Beck et al. 2009). A 78 dpt double aspheric lens (Volk Optical, Inc., Mentor, OH 44060, U.S.A.) was placed directly to the outlet of the device, an additional custom-made+3.5 dpt contact lens directly on the eyes of the rats. The rats were anaesthetized, the pupils dilated and treated with Methocel to avoid drying of the eyes and for better adherence of the 3.5 dpt lens. The ICG dye (250 μl (VERDYE, 5 mg/ml, Diagnostic Green) was injected into the tail vein, the fluorescein dye (Alcon 10% (1/10 dilution), 250 μl) was injected subcutaneously. SLO/OCT was performed ca. 2 to 5 minutes after injection for early phase and ca 15 to 20 minutes later for late phase angiography imaging. As the SLO/OCT machine is calibrated for the use with human eyes, the dimension in the x and y axis are not corrected for use in rats. Dimensions in the z axis, like retinal height, are displayed properly. Therefore, measurements of CNV hyper-fluorescent areas in the angiography measurements performed here are presented in arbitrary units (au) and not in μm using the original Heidelberg calibration. Quantification of the thickness measurements performed in OCT data sets is displayed in μm as they lay in the z direction of the beam.

Processing of the Eyes for Histology

For electron microscopy (EM), whole eyes were fixed in 5% glutaraldehyde in 0.1 M cacodylate buffer (pH 7.4) over night. Then, it will be possible to cut the lesion area, as it was visible in the angiography, and to embed it.

Statistics

Students t-test was performed to compare the results of the treated animals with the control groups. For the analysis the excel software was used. The error probability was 5% (p<0.05 statistically significant). To avoid the multiple comparisons problem, the results were corrected using the Holm-Bonferroni method.

Investigation of CNV 6 Weeks after Subretinal Injection of AAV.VEGF-A165 in Rat Eyes by Angiography

The AAV-VEGF triggered rat CNV model showed a fully grown CNV 6 weeks after VEGF transduction, as documented by in vivo imaging. A representative image is presented in (see, FIG. 8).

All 60 eyes overexpressing VEGF showed typical CNV lesion-like hyper-fluorescence in FA and ICG imaging (FIG. 9) meaning that the VEGF transduction efficacy was 100%.

In the following, eyes successfully transduced with VEGF vector and showing CNV-like lesions will be termed “CNV eyes”, the CNV-like lesions “CNV lesion”.

All eyes were investigated by angiography. Most CNV lesions showed a typical ring-shaped pattern in both the FA and ICG angiographies. A central hypo-fluorescent area was surrounded by a bright hyper-fluorescent ring especially in FA images (see, FIG. 9 left panel). This pattern correlated well with the OCT analyses showing pronounced subretinal lesions in the hyper-reflective areas. In contrast, the ICG signal showed a rather spotty pattern that usually stretched over a larger area around the hypo-fluorescent center of the lesion.

ICG is a dye that has a very long half-life and binds to luminal proteins. Therefore, it can be recorded at several time points after single intravenous injection if it is retained within the tissue. This occurs, e.g., with protein leakage from CNV vessels into the surrounding tissue.

As shown in FIG. 9 (see, right panel, green channel) and in contrast to the FA signal, the ICG hyper-fluorescence shows a rather spotty pattern around the CNV lesion that spreads over time (within 20 minutes, but also at reinvestigation of ICG without additional dye injections at later time points, here one week after the first angiography session). Finally, this leads to formation of larger fields of single hyper-fluorescent highlights that can cover the whole background of the eye at late time points. These patterns, however, do not dramatically change directly after injection of additional ICG dye.

Reduction of the Thickness of the CNV Lesion Area by PEDF Treatment

For each eye the area of the whole CNV lesion area (detected by SLO angiography) was screened by OCT. The area of maximal thickness of the lesion was determined and imaged. In these images the maximal thickness was measured. To analyze the changes caused by the treatment with the different agents the differences of the measurement values for each eye for the analysis seven weeks after the subretinal injection of the vector (one week after treatment) and the corresponding values for the six weeks analysis (before treatment) were determined. PEDF inhibited cellular proliferation and fibrosis and therefore reduced the thickness of the CNV significantly compared to the untreated group, but the blood vessels did not collapse completely as in the Avastin group. Thus, the CNVs became flatter in the Avastin group (see, FIG. 10).

Effects of PEDF on New Formation of a Healthy Choriocapillaris, Bruch's Membrane and Junctional Complexes

Eyes after PEDF protein treatment were investigated by electron microscopy and eyes after VEGF vector injection without PEDF treatment were used as controls. The most prominent effects of PEDF treatment were that the newly formed choriocapillaris was very similar to the healthy choriocapillaris without any treatment. The newly formed vessels were directly located below the RPE and formed a new Bruch's membrane (see, FIG. 11). The endothelial cells were thin as in healthy vessels, were associated with pericytes, developed fenestrations (see, FIG. 12) and did not grow into the subretinal space.

In addition, junctional complexes between retinal pigment epithelial cells (FIG. 14) and the endothelial cells of the choriocapillaris (FIG. 15) were dramatically enlarged and electron dense compared to only VEGF vector treated eyes. These complexes consist of adherent junctions and tight junctions. Tight junctions also appeared between endothelial cells of the choriocapillaris although they have not been reported in these vessels before. It is generally accepted that the blood retina barrier is built up by the tight junctions of the retinal vessels and the tight junctions of the retinal pigment epithelium. The effect on the junctions is mediated by PEDF in combination with VEGF over expression.

PEDF also reduced dividing of RPE cells and inhibited the formation of intravascular protrusions containing extracellular matrix (see, FIGS. 2 and 15). This phenomenon was also described in a rabbit model of CNV (Julien, Kreppel et al. 2008). Such protrusions were not seen after PEDF treatment, which caused formation of monolayered basement membranes in newly formed vessel whereas without treatment the basement membranes were multilayered. Also, the breakthrough of newly formed blood vessels into the subretinal space and retina did not occur after PEDF treatment but were seen without PEDF injection.

### EXAMPLE 7: EFFECT OF A COMBINATION OF PEDF AND ANTI-VEGF DRUG

A combination of PEDF and an anti-VEGF drug, for example Bevacizumab (Avastin), acts synergistically and is supporting the coordinated growth of new functional vessels and also improves the formation of fenestrations in the newly formed choriocapillaris.

### EXAMPLE 8: PEDF REDUCES FORMATION OF EXTRACELLULAR MATRIX IN CNV

As shown in this example, PEDF reduced formation of extracelluar matrix in CNV. Therefore, scarring which is typical for CNV is minimized and therefore the distance for supply of oxygen and nutrition from the newly formed vessels towards the PRE and photoreceptors was shortened.

Methods

A subretinal injection of 2 μl AAV.VEGF-A¹⁶⁵ (2×10⁹ virus particles of the AAV-VEGF vector, diluted in 2 μl PBS) was performed in 48 eyes of Long Evans rats in order to induce a CNV. After three weeks, the CNV development was checked by in vivo examinations and 100% of the eyes (n=48 eyes) showed a CNV.

Directly after the in vivo investigation, the eyes were intravitreally treated with 4 μl of PEDF protein (10 μg) “group 1” (n=12 eyes) or avastin (50 μg) “group 2” (n=12 eyes) or as combination therapy (PEDF protein (10 μg)+avastin (50 μg)) “group 3” (n=12 eyes). Untreated eyes served as control “group 4” (n=12 eyes).

At week 6 a second intravitreal treatment of PEDF protein or avastin or a combination of both proteins was performed as described for the week 3. One week later at week 7, the effect on maturation of the extracellular matrix in the CNV was evaluated by polarization microscopy.

Paraffin sections of the eyes were stained according to the following protocol.

Picrosirius Red Stain Protocol
1. Deparaffinize and hydrate in distilled water
2. Stain in Weigerts Hematoxylin for 8 minutes
3. Rinse well in distilled water
4. Place in solution A for 2 minutes
5. Distilled water rinse
6. Place in solution B for 60 minutes
7. Place in solution C for 2 minutes
8. 70% ethanol for 45 seconds
9. Dehydrate, clear and mount
10. Slides are evaluated under the polarized microscope (Axioplan, Zeiss). This method allows to discriminate different types of collagen by colour. Type I (Red, Orange); type III (Yellow, Green).

Results

The results are shown In FIG. 16.

Within the area of choroidal neovascularization collagen appeared green under the polarizing microscope after the injection of PEDF and Avastin (FIG. 16, left column). Also, after injection of avastin alone the collagen was greenish but the amount of collagen was largely enhanced (FIG. 16, middle column) compared to injection of both proteins. The green color indicated that the collagen was type III which is typical for fibrotic tissues. After injection of PEDF alone the collagen was orange and surrounded the blood vessels as a thin layer (FIG. 16, arrow, right column). This indicated that the collagen had matured and the new formation of the extracellular matrix and vessels had stopped. Greenish collagen was not seen after the specimen was turned by 360 degree after PEDF injection. Without treatment the collagen was greenish and occupied the majority of the CNV area (not shown) similar to the results after avastin injection (FIG. 16, middle column).

### EXAMPLE 9: MIMICKING HUMAN AMD BY SUBRETINAL OR INTRAVITRAL INJECTION OF VEGF

Hundred ng VEGF protein (hVEGF Sigma) in 2 μl PBS were injected subretinally or intravitreally into eyes of Long Evans rats. For controls, only PBS was injected.

The eyes were investigated after 1 and 24 hours by electron microscopy and immunocytchemistry. The choriocapillaris changed into labyrinth capillaries as shown in FIGS. 2 to 4 and an earlier publication in human CNV's (Schraermeyer, Julien et al. 2015). In addition, there was a prominent augmentation of the extracellular matrix within Bruch's membrane and around the choricapillaris. The protrusion of extracellular matrix which induced the endothelial invaginations into the vessel lumen as shown in FIG. 15 was also present. The synthesis of basement membranes of RPE and vessels was enhanced to multilayers. In addition, the RPE was highly activated and migrated out of the monolayer. Within the choriocapillaris, thrombocytes were activated red blood cells were lysed probably by complement activation and also developed stasis. All these findings were surprisingly already observed 1-24 hours after injection, lacked in the control group and mimicked the findings seen in human eyes suffering from AMD.

### EXAMPLE 10: IN VITRO EFFECTS OF PEDF, BEVACIZUMAB OR A COMBINATION OF BOTH PEDF AND BEVACIZUMAB ON ANGIOGENESIS

In vitro effects of PEDF, Bevacizumab (Avastin) or a combination of both PEDF and Bevacizumab (Avastin) on angiogenesis were determined in an endothelial cell tube formation assay. The endothelial cell tube formation assay is a classical in vitro assay to study angiogenesis and anti-angiogenic effects of potential drug candidates.

Methods: Endothelial Cell Tube Formation Assay

96-well plates (Corning, USA) were pre-coated with 60 μL of growth factor reduced Matrigel (BD Biosciences, USA), and HUVEC cells (13000 cells/well) in ECGM Media (Promocell, Germany) were seeded onto the plates. The wells were supplemented with: PEDF alone (250 ng/ml, 500 ng/ml), Bevacizumab alone (Avastin; Genentech, Inc., South San Francisco, CA) (250 μg/mL, 1 mg/mL, 2 mg/mL) and together at a concentration of PEDF (250 ng/mL)+Bevacizumab (250 μg/mL) and PEDF (250 ng/mL)+Bevacizumab (1 mg/mL) to determine the effects of these molecules on endothelial cell tube formation. After incubation for 5 hrs at 37° C. the tube formation was analysed in the wells using a Leica DM IL LED inverted phase contrast microscope.

Results

The results are shown in FIGS. 17a-h.

There was only little inhibition of endothelial tube formation with PEDF at a concentration of 250 ng/mL (FIG. 17b) with a complete inhibition observed at 500 ng/mL (FIG. 17c). Bevacizumab inhibited tube formation only at a concentration of 2 mg/mL (FIG. 17f). Co-administration of PEDF and Bevacizumab at concentrations of 250 ng/mL (PEDF) and 250 μg/mL (Bevacizumab), respectively, showed a much stronger inhibitory effect on tube formation (FIG. 17g) than when individually treated with PEDF or Bevacizumab at the same concentrations. This was particularly evident for Bevacizumab which, when used alone, inhibited endothelial tube formation only at a high concentration of 2 mg/mL. Thus, Bevacizumab was thus effective in inhibiting tube formation at a much lower concentration when treated in combination with PEDF (FIGS. 17b and 17h). This data indicates a synergistic effect of PEDF and Bevacizumab with respect to the inhibition of endothelial tube formation and thus in the inhibition of angiogenesis.

### EXAMPLE 11: PEDF INDUCES GROWTH OF CHORIOCAPILLARIS

This example shows that PEDF induce growth of choriocapillaris as evidenced in a model for dry AMD using sodium iodate (NaIO3)⁻.

The sodium iodate (NaIO3)-induced model of retinal degeneration has been shown to display some dry AMD-associated features (Hanus, J., et al. (2016). “Retinal pigment epithelial cell necroptosis in response to sodium iodate.” Cell Death Discov 2: 16054).

In LE rats, the intravenous dose of 40 mg/kg NaIO3 has been shown to be optimal to induce regional and time dependent alteration of the RPE. Here, the efficacy of the treatment of PEDF protein alone (11 μg) or in combination with an anti-VEGF agent such as Avastin (50 μg) following a single intravitreal injection given directly after the NaIO3 intoxication is shown.

In vivo examinations and flat-mounted RPE/choroid complexes show formation of new healthy choriocapillaries three days later.

### EXAMPLE 12: PEDF IN COMBINATION WITH VEGF INDUCES GROWTH OF CHORIOCAPILLARIS

This example shows that PEDF in combination with VEGF induce growth of choriocapillaris as evidenced in a dry AMD model using sodium iodate (NaIO3).

The sodium iodate (NaIO3)-induced model of retinal degeneration has been shown to display some dry AMD-associated features. In LE rats, the intravenous dose of 40 mg/kg NaIO3 has been shown to be optimal to induce regional and time dependent alteration of the RPE. Here, the efficacy of the treatment of PEDF protein (11 μg) alone or in combination with VEGF (2-4 μg) protein following a single intravitreal injection given directly after the NaIO3 intoxication is shown. Both proteins are also injected separately.

In vivo examinations and flat-mounted RPE/choroid complexes show formation of new healthy choriocapillaries three days later. The new choriocapillaries are healthy, i.e. non-leaking, and functional when PEDF and VEGF are combined or PEDF is used alone.

### EXAMPLE 13: PEDF IN COMBINATION WITH AN ANTI VEGF DRUG INDUCES GROWTH OF HEALTHY RETINAL VESSELS

This example shows that PEDF in combination with an anti-VEGF drug induce growth of healthy retinal vessels as evidenced in a neovascularization model using an oxygen-induced ischemic retinopathy model.

PEDF protein alone (11 μg) or combined with an anti-VEGF antibody (Avastin, 50 μg) in a newborn rat oxygen-induced ischemic retinopathy (OIR) induce formation of new retinal blood vessels.

Said model is considered representative of the retinopathy of prematurity (ROP) and was already described by Semkova I et al. (Semkova, I., et al. (2002)). “Autologous transplantation of genetically modified iris pigment epithelial cells: a promising concept for the treatment of age-related macular degeneration and other disorders of the eye.” Proc Natl Acad Sci USA 99(20): 13090-13095).

Briefly, rats are maintained in room air for the normoxia group, whereas the rats in the hyperoxia group are exposed to 75% oxygen from postnatal day 7 (P7) to P12, then they are removed to room air and immediately treated.

The non-perfused areas and degree of vascularization is determined as described in Semkova et al (2002) (supra). The new retinal vessels are healthy, i.e. non-leaking, and functional when PEDF is used alone of used in combination with an anti-VEGF drug.

### EXAMPLE 14: PEDF IN COMBINATION WITH ANTI-VEGF REDUCED THE AMOUNT OF VEGF MORE EFFECTIVE THAN PEDF ALONE

Choroidal neovascularizations (CNVs) produced in the experiments described in Example 6 were stained with anti-VEGF antibodies in paraffin sections. 30 rats were subretinally injected with AAV-VEGF vectors. Three weeks after that, all of them developed CNV around the area of injection. The rats were divided in 5 groups. The total CNV area and the VEGF positive area were measured using the FIJI software, using a semi-automated machine learning based approach (WEKA segmentation).

The results are shown in FIGS. 18 and 19.

As illustrated in FIG. 19, treatment with PEDF (11 μg)+Avastin (50 μg) (n=6) resulted in a significant reduction of VEGF in the CNV area in this group compared to the untreated control group (n=5). Treatment with PEDF alone, resulted in strong intra-individual variation of VEGF in this group (n=5). Compared to this PEDF group, the Avastin+PEDF group also showed a significant reduction. Data was analyzed by Wilcoxon test with Kruskal-Wallis post-test. (* p<0.05). Although the combination of PEDF and Avastin was only minimal more effective than Avastin alone, the combination is of advantage due the anti-apoptotic effect of PEDF (see example 15).

### EXAMPLE 15: NEUROPROTECTIVE EFFECT OF PEDF

This example shows that PEDF is neuroprotective as evidenced in a hypoxia rat eye model.

Hypoxic retinae were produced as described in example 1. Rat eyes were isolated and immediately injected with PBS (vehicle) or PEDF solution (11.5 μg/μl). After 14 h incubation in the refrigerator (4° C.), the eyes were fixed and embedded in paraffin. The Tunnel-analysis was performed using the in situ cell death detection TMR red TUNEL kit (Roche Diagnostics, Mannheim, Germany) as recommended by the manufacturer. The apoptotic cell nuclei were shown in red; the outer segments of the photoreceptors also appeared red, but this was caused by retinal fluorophores.

The results are shown in FIGS. 20 and 21.

The treatment with PEDF reduced the number of apoptotic cells in the ex vivo retinal hypoxia model described in example 1 and this provided a significant neuroprotective effect on ganglion cells compared with vehicle treatment. (* * p<0.05).

### REFERENCES

The complete bibliographic data of the documents recited herein the disclosure of which is incorporated by reference is, if not indicated to the contrary, as follows.
Biesemeier, A., T. Taubitz, S. Julien, E. Yoeruek and U. Schraermeyer (2014). “Choriocapillaris breakdown precedes retinal degeneration in age-related macular degeneration.” Neurobiol Aging 35(11): 2562-2573.
Biesemeier, A. K., S. Julien and U. Schraermeyer (2014a). “Choroidal neovascularization can help photoreceptors to survive in late AMD.” Investigative Ophthalmology & Visual Science 55(13).
Browning, D. J., P. K. Kaiser, P. J. Rosenfeld and M. W. Stewart (2012). “Aflibercept for age-related macular degeneration: a game-changer or quiet addition?” Am J Ophthalmol 154(2): 222-226.
Daha, N. A., N. K. Banda, A. Roos, F. J. Beurskens, J. M. Bakker, M. R. Daha and L. A. Trouw (2011). “Complement activation by (auto-) antibodies.” Mol Immunol 48(14): 1656-1665.
Ferrara, N., L. Damico, N. Shams, H. Lowman and R. Kim (2006). “Development of ranibizumab, an anti-vascular endothelial growth factor antigen binding fragment, as therapy for neovascular age-related macular degeneration.” Retina 26(8): 859-870.
Fischer, M. D., G. Huber, S. C. Beck, N. Tanimoto, R. Muehlfriedel, E. Fahl, C. Grimm, A. Wenzel, C. E. Reme, S. A. van de Pavert, J. Wijnholds, M. Pacal, R. Bremner and M. W. Seeliger (2009). “Noninvasive, in vivo assessment of mouse retinal structure using optical coherence tomography.” PLoS One 4(10): e7507.
Green W et al.; Trans Am Ophthalmol Soc. 1977; 75: 180) and Sarks S et al. (Br J Ophthalmol 1973, 57:951
Hanhart, J., D. S. Comaneshter, Y. Freier Dror and S. Vinker (2017). “Mortality in patients treated with intravitreal bevacizumab for age-related macular degeneration.” BMC Ophthalmol 17(1): 189.
Hanhart, J., D. S. Comaneshter, Y. Freier-Dror and S. Vinker (2018). “Mortality associated with bevacizumab intravitreal injections in age-related macular degeneration patients after acute myocardial infarct: a retrospective population-based survival analysis.” Graefes Arch Clin Exp Ophthalmol 256(4): 651-663.
Hanhart, J., D. S. Comaneshter and S. Vinker (2018). “Mortality after a cerebrovascular event in age-related macular degeneration patients treated with bevacizumab ocular injections.” Acta Ophthalmol 96(6): e732-e739.
Hanus, J., et al. (2016). “Retinal pigment epithelial cell necroptosis in response to sodium iodate.” Cell Death Discov 2: 16054.
He, T., J. Hu, G. Yan, L. Li, D. Zhang, Q. Zhang, B. Chen and Y. Huang (2015). “Pigment epithelium-derived factor regulates microvascular permeability through adipose triglyceride lipase in sepsis.” Clin Sci (Lond) 129(1): 49-61.
Huber, G., S. C. Beck, C. Grimm, A. Sahaboglu-Tekgoz, F. Paquet-Durand, A. Wenzel, P. Humphries, T. M. Redmond, M. W. Seeliger and M. D. Fischer (2009). “Spectral domain optical coherence tomography in mouse models of retinal degeneration.” Invest Ophthalmol Vis Sci 50(12): 5888-5895.
Julien, S., A. Biesemeier and U. Schraermeyer (2013). “In vitro induction of protein complexes between bevacizumab, VEGF-A(1)(6)(5) and heparin: explanation for deposits observed on endothelial veins in monkey eyes.” Br J Ophthalmol 97(4): 511-517.
Julien, S., A. Biesemeier, T. Taubitz and U. Schraermeyer (2014). “Different effects of intravitreally injected ranibizumab and aflibercept on retinal and choroidal tissues of monkey eyes.” Br J Ophthalmol 98(6): 813-825.
Julien, S., F. Kreppel, S. Beck, P. Heiduschka, V. Brito, S. Schnichels, S. Kochanek and U. Schraermeyer (2008). “A reproducible and quantifiable model of choroidal neovascularization induced by VEGF A165 after subretinal adenoviral gene transfer in the rabbit.” Mol Vis 14: 1358-1372.
King, G. L. and K. Suzuma (2000). “Pigment-epithelium-derived factor—A key coordinator of retinal neuronal and vascular functions.” New England Journal of Medicine 342(5): 349-351.
Mentes, J. and S. Yildirim (2019). “Optical Coherence Tomography Characteristics of Quiescent Type 1 Neovascularization in Eyes with Nonexudative Age-related Macular Degeneration.” Turk J Ophthalmol 49(2): 84-88.
Meyer, C. H. and F. G. Holz (2011). “Preclinical aspects of anti-VEGF agents for the treatment of wet AMD: ranibizumab and bevacizumab.” Eye (Lond) 25(6): 661-672.
Meyer, T., L. Robles-Carrillo, T. Robson, F. Langer, H. Desai, M. Davila, M. Amaya, J. L. Francis and A. Amirkhosravi (2009). “Bevacizumab immune complexes activate platelets and induce thrombosis in FCGR2A transgenic mice.” J Thromb Haemost 7(1): 171-181.
Muller, P. L., et al. (2020). OCT-Angiography in Geographic Atrophy. Ophthalmologica; doi: 10.1159/000510727
Papadopoulos, N., J. Martin, Q. Ruan, A. Rafique, M. P. Rosconi, E. Shi, E. A. Pyles, G. D. Yancopoulos, N. Stahl and S. J. Wiegand (2012). “Binding and neutralization of vascular endothelial growth factor (VEGF) and related ligands by VEGF Trap, ranibizumab and bevacizumab.” Angiogenesis 15(2): 171-185.
Peters, S., P. Heiduschka, S. Julien, F. Ziemssen, H. Fietz, K. U. Bartz-Schmidt, G. Tubingen Bevacizumab Study and U. Schraermeyer (2007). “Ultrastructural findings in the primate eye after intravitreal injection of bevacizumab.” Am J Ophthalmol 143(6): 995-1002.
Querques et al. (2013) Functional Characterization and Multimodal Imaging of Treatment-Na\ddot{v}e “Quiescent” Choroidal Neovascularization IOVS j October j Vol. 54 j No. 10 j 6887.
Rolling, F., G. Le Meur, K. Stieger, A. J. Smith, M. Weber, J. Y. Deschamps, D. Nivard, A. Mendes-Madeira, N. Provost, Y. Pereon, Y. Cherel, R. R. Ali, C. Hamel, P. Moullier and F. Rolling (2006). “Gene therapeutic prospects in early onset of severe retinal dystrophy: restoration of vision in RPE65 Briard dogs using an AAV serotype 4 vector that specifically targets the retinal pigmented epithelium.” Bull Mem Acad R Med Belg 161(10-12): 497-508; discussion 508-499.
Sarks S et al.; Br J Ophthalmol 1973, 57:951
Schraermeyer, U. and S. Julien (2012). “Formation of immune complexes and thrombotic microangiopathy after intravitreal injection of bevacizumab in the primate eye.” Graefes Arch Clin Exp Ophthalmol 250(9): 1303-1313.
Schraermeyer, U. and S. Julien (2013). “Effects of bevacizumab in retina and choroid after intravitreal injection into monkey eyes.” Expert Opin Biol Ther 13(2): 157-167.
Schraermeyer, U., S. Julien, A. Biesemeier, K. U. Bartz-Schmidt and H. Wolburg (2015). “A new kind of labyrinth-like capillary is responsible for leakage from human choroidal neovascular endothelium, as investigated by high-resolution electron microscopy.” Graefes Arch Clin Exp Ophthalmol 253(5): 681-689.
Schutze, C., M. Wedl, B. Baumann, M. Pircher, C. K. Hitzenberger and U. Schmidt-Erfurth (2015). “Progression of retinal pigment epithelial atrophy in antiangiogenic therapy of neovascular age-related macular degeneration.” Am J Ophthalmol 159(6): 1100-1114 e1101.
Semkova, I., et al. (2002)). “Autologous transplantation of genetically modified iris pigment epithelial cells: a promising concept for the treatment of age-related macular degeneration and other disorders of the eye.” Proc Natl Acad Sci USA 99(20): 13090-13095.
Spaide R F, Klancnik J M, Cooney M J. Retinal Vascular Layers Imaged by Fluorescein Angiography and Optical Coherence Tomography Angiography. JAMA Ophthalmol. 2015; 133(1):45.
Treister, A. D., P. L. Nesper, A. E. Fayed, M. K. Gill, R. G. Mirza and A. A. Fawzi (2018). “Prevalence of Subclinical CNV and Choriocapillaris Nonperfusion in Fellow Eyes of Unilateral Exudative AMD on OCT Angiography.” Transl Vis Sci Technol 7(5): 19.
Wang, F., K. G. Rendahl, W. C. Manning, D. Quiroz, M. Coyne and S. S. Miller (2003). “AAV-mediated expression of vascular endothelial growth factor induces choroidal neovascularization in rat.” Invest Ophthalmol Vis Sci 44(2): 781-790.
Yannuzzi et al.; Retina, 1992; 12:191-223

The features of the present invention disclosed in the specification, the claims, the sequence listing and/or the drawings may both separately and in any combination thereof be material for realizing the invention in various forms thereof.

1. A pigment epithelium-derived factor (PEDF) for use in a method for the treatment and/or prevention of dry macular degeneration in a subject, preferably dry age-related macular degeneration, wherein the method comprises administering PEDF and VEGF to the subject, wherein the PEDF and the VEGF are administered to the subject concurrently or sequentially.

2. The pigment epithelium-derived factor (PEDF) for use of claim 1, wherein the subject is suffering from treatment-naïve quiescent choroidal neovascularization.

3. The pigment epithelium-derived factor (PEDF) for use of claim 1, wherein the subject is suffering from geographic atrophy.

4. The pigment epithelium-derived factor (PEDF) for use of claim 3, wherein geographic atrophy is the result of anti-VEGF therapy.

5. The pigment epithelium-derived factor (PEDF) for use of claim 1, wherein PEDF induces growth of choriocapillaris.

6. The pigment epithelium-derived factor (PEDF) for use of claim 5, wherein choriocapillaris are non-leaking choriocapillaris.

7. The pigment epithelium-derived factor (PEDF) for use of claim 1, wherein PEDF inhibits growth and/or formation of geographic atrophy.

8. A pigment epithelium-derived factor (PEDF) for use in a method for the treatment and/or prevention of wet macular degeneration in a subject, preferably wet age-related macular degeneration, wherein the method comprises administering PEDF and an anti-VEGF therapy to the subject, wherein the PEDF and the anti-VEGF therapy are administered to the subject concurrently or sequentially.

9. The pigment epithelium-derived factor (PEDF) for use of claim 8, wherein PEDF induces growth of non-leaking choriocapillaris.

10. The pigment epithelium-derived factor (PEDF) for use of claim 8, wherein the anti-VEGF therapy comprises administration of an anti-VEGF drug to the subject.

11. The pigment epithelium-derived factor (PEDF) for use of claim 8, wherein the anti-VEGF drug is selected from the group comprising pegaptanib, ranibizumab, bevacizumab and aflibercept.

12. A pigment epithelium-derived factor (PEDF) for use in a method for the treatment and/or prevention of treatment-naïve quiescent choroidal neovascularization in a subject, wherein the method comprises administering PEDF and VEGF to the subject, wherein the PEDF and the VEGF are administered to the subject concurrently or sequentially.

13. The pigment epithelium-derived factor (PEDF) for use of claim 12, wherein the subject is suffering from dry macular degeneration, preferably dry age-related macular degeneration.

14. The pigment epithelium-derived factor (PEDF) for use of claim 12, wherein the subject is suffering from geographic atrophy.

15. The pigment epithelium-derived factor (PEDF) for use of claim 14, wherein geographic atrophy is the result of anti-VEGF therapy.

16. The pigment epithelium-derived factor (PEDF) for use of claim 12, wherein PEDF induces growth of choriocapillaris.

17. The pigment epithelium-derived factor (PEDF) for use of claim 16, wherein the choriocapillaris are non-leaking choriocapillaris.

18. The pigment epithelium-derived factor (PEDF) for use of claim 12, preferably any one of claims 14, 15, 16 and 17, wherein PEDF inhibits growth and/or formation of geographic atrophy.


SEQUENCE LISTING (47 sequences)

SEQ ID NO: 1 - protein, 399 aa, Homo sapiens
QNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 2 - RNA, 1257 nt, Homo sapiens
augcaggcccuggugcuacuccucugcauuggagcccuccucgggcacagcagcugccagaacccugccagccccccggaggagggcuccccagaccccgacagcacaggggcgcugguggaggaggaggauccuuucuucaaaguccccgugaacaagcuggcagcggcugucuccaacuucggcuaugaccuguaccgggugcgauccagcacgagccccacgaccaacgugcuccugucuccucucaguguggccacggcccucucggcccucucgcugggagcggagcagcgaacagaauccaucauucaccgggcucucuacuaugacuugaucagcagcccagacauccaugguaccuauaaggagcuccuugacacggucaccgccccccagaagaaccucaagagugccucccggaucgucuuugagaagaagcugcgcauaaaauccagcuuuguggcaccucuggaaaagucauaugggaccaggcccagaguccugacgggcaacccucgcuuggaccugcaagagaucaacaacugggugcaggcgcagaugaaagggaagcucgccagguccacaaaggaaauucccgaugagaucagcauucuccuucucgguguggcgcacuucaaggggcaguggguaacaaaguuugacuccagaaagacuucccucgaggauuucuacuuggaugaagagaggaccgugaggguccccaugaugucggacccuaaggcuguuuuacgcuauggcuuggauucagaucucagcugcaagauugcccagcugcccuugaccggaagcaugaguaucaucuucuuccugccccugaaagugacccagaauuugaccuugauagaggagagccucaccuccgaguucauucaugacauagaccgagaacugaagaccgugcaggcgguccucacuguccccaagcugaagcugaguuacgaaggcgaagucaccaagucccugcaggagaugaagcugcaauccuuguuugauucaccagacuuuagcaagaucacaggcaaacccaucaagcugacucagguggaacaccgggcuggcuuugaguggaacgaggauggggcgggaaccacccccagcccagggcugcagccugcccaccucaccuucccgcuggacuaucaccuuaaccagccuuucaucuucguacugagggacacagacacaggggcccuucucuucauuggcaagauucuggaccccaggggccccuaa

SEQ ID NO: 3 - RNA, 1000 nt, Homo sapiens
ggacgcuggauuagaaggcagcaaaaaaagaucugugcuggcuggagcccccucagugugcaggcuuagagggacuaggcuggguguggagcugcagcguauccacaggccccaggaugcaggcccuggugcuacuccucugcauuggagcccuccucgggcacagcagcugccagaacccugccagccccccggaggagggcuccccagaccccgacagcacaggggcgcugguggaggaggaggauccuuucuucaaaguccccgugaacaagcuggcagcggcugucuccaacuucggcuaugaccuguaccgggugcgauccagcaugagccccacgaccaacgugcuccugucuccucucaguguggccacggcccucucggcccucucgcugggagcggacgagcgaacagaauccaucauucaccgggcucucuacuaugacuugaucagcagcccagacauccaugguaccuauaaggagcuccuugacacggucacugccccccagaagaaccucaagagugccucccggaucgucuuugagaagaagcurcgcauaaaauccagcuuuguggcaccucuggaaaagucauaugggaccaggcccagaguccugacgggcaacccucgcuuggaccugcaagagaucaacaacugggugcaggcgcagaugaaagggaagcucgccagguccacaaaggaaauucccgaugagaucagcauucuccuucucgguguggcgcacuucaaggggcaguggguaacaaaguuugacuccagaaagacuucccucgaggauuucuacuuggaugaagagaggaccgugaggguccccaugaugucggacccuaaggcuguuuuacgcuauggcuuggauucagaucucagcugcaagauugcccagcugcccuugaccggaagcaugaguaucaucuucuuccugccccugaaagugacccagaauuugaccuugauagaggagagccucaccuc

SEQ ID NO: 4 - protein, 412 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 5 - RNA, 3660 nt, Homo sapiens
gcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaaaaaucaguucgaggaaagggaaaggggcaaaaacgaaagcgcaagaaaucccgguauaaguccuggagcguguacguuggugcccgcugcugucuaaugcccuggagccucccuggcccccaucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaa

SEQ ID NO: 6 - protein, 395 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 7 - RNA, 3609 nt, Homo sapiens
gcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaaaaaucaguucgaggaaagggaaaggggcaaaaacgaaagcgcaagaaaucccgguauaaguccuggagcguucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaa

SEQ ID NO: 8 - protein, 389 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRR

SEQ ID NO: 9 - RNA, 3591 nt, Homo sapiens
gcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaaaaaucaguucgaggaaagggaaaggggcaaaaacgaaagcgcaagaaaucccgucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaa

SEQ ID NO: 10 - protein, 371 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 11 - RNA, 3537 nt, Homo sapiens
gcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaa

SEQ ID NO: 12 - protein, 354 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKM

SEQ ID NO: 13 - RNA, 3502 nt, Homo sapiens
gcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaucccugugggccuugcucagagcggagaaagcauuuguuuguacaagauccgcagacguguaaauguuccugcaaaaacacagacucgcguugcaagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaa

SEQ ID NO: 14 - protein, 327 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 15 - RNA, 3405 nt, Homo sapiens
gcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaa

SEQ ID NO: 16 - protein, 371 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRSLTRKD

SEQ ID NO: 17 - RNA, 3471 nt, Homo sapiens
gcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaucccugugggccuugcucagagcggagaaagcauuuguuuguacaagauccgcagacguguaaauguuccugcaaaaacacagacucgcguugcaaggcgaggcagcuugaguuaaacgaacguacuugcagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaa

SEQ ID NO: 18 - protein, 317 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRCDKPRR

SEQ ID NO: 19 - RNA, 3375 nt, Homo sapiens
gcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaa

SEQ ID NO: 20 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 21 - RNA, 3677 nt, Homo sapiens
ucgcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaaaaaucaguucgaggaaagggaaaggggcaaaaacgaaagcgcaagaaaucccgguauaaguccuggagcguguacguuggugcccgcugcugucuaaugcccuggagccucccuggcccccaucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaaaaaaaaaaaaaaaaa

SEQ ID NO: 22 - protein, 215 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 23 - RNA, 3626 nt, Homo sapiens
ucgcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaaaaaucaguucgaggaaagggaaaggggcaaaaacgaaagcgcaagaaaucccgguauaaguccuggagcguucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaaaaaaaaaaaaaaaaa

SEQ ID NO: 24 - protein, 209 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRR

SEQ ID NO: 25 - RNA, 3608 nt, Homo sapiens
ucgcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaaaaaucaguucgaggaaagggaaaggggcaaaaacgaaagcgcaagaaaucccgucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaaaaaaaaaaaaaaaaa

SEQ ID NO: 26 - protein, 174 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKM

SEQ ID NO: 27 - RNA, 3519 nt, Homo sapiens
ucgcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaucccugugggccuugcucagagcggagaaagcauuuguuuguacaagauccgcagacguguaaauguuccugcaaaaacacagacucgcguugcaagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaaaaaaaaaaaaaaaaa

SEQ ID NO: 28 - protein, 147 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 29 - RNA, 3422 nt, Homo sapiens
ucgcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaaaaaaaaaaaaaaaaa

SEQ ID NO: 30 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRSLTRKD

SEQ ID NO: 31 - RNA, 3488 nt, Homo sapiens
ucgcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaucccugugggccuugcucagagcggagaaagcauuuguuuguacaagauccgcagacguguaaauguuccugcaaaaacacagacucgcguugcaaggcgaggcagcuugaguuaaacgaacguacuugcagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaaaaaaaaaaaaaaaaa

SEQ ID NO: 32 - protein, 171 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVCDKPRR

SEQ ID NO: 33 - RNA, 3494 nt, Homo sapiens
ucgcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaaaaaucaguucgaggaaagggaaaggggcaaaaacgaaagcgcaagaaaucccgguauaaguccuggagcguaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaaaaaaaaaaaaaaaaa

SEQ ID NO: 34 - protein, 351 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVCDKPRR

SEQ ID NO: 35 - RNA, 3477 nt, Homo sapiens
gcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaaaaaucaguucgaggaaagggaaaggggcaaaaacgaaagcgcaagaaaucccgguauaaguccuggagcguaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaa

SEQ ID NO: 36 - protein, 163 aa, Homo sapiens
MAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 37 - RNA, 2569 nt, Homo sapiens
gcggagccgauuacaucagcccgggccuggccggccgcguguucccggagccucggcugcccgaauggggagcccagaguggcgagcggcaccccuccccccgccagcccuccgcgggaaggugaccucucgaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaa

SEQ ID NO: 38 - protein, 213 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRRSAGQEEGASLRVSGTRSLTRKD

SEQ ID NO: 39 - RNA, 3554 nt, Homo sapiens
ucgcggaggcuuggggcagccggguagcucggaggucguggcgcugggggcuagcaccagcgcucugucgggaggcgcagcgguuagguggaccggucagcggacucaccggccagggcgcucggugcuggaauuugauauucauugauccggguuuuaucccucuucuuuuuucuuaaacauuuuuuuuuaaaacuguauuguuucucguuuuaauuuauuuuugcuugccauuccccacuugaaucgggccgacggcuuggggagauugcucuacuuccccaaaucacuguggauuuuggaaaccagcagaaagaggaaagagguagcaagagcuccagagagaagucgaggaagagagagacggggucagagagagcgcgcgggcgugcgagcagcgaaagcgacaggggcaaagugagugaccugcuuuugggggugaccgccggagcgcggcgugagcccucccccuugggaucccgcagcugaccagucgcgcugacggacagacagacagacaccgcccccagccccagcuaccaccuccuccccggccggcggcggacaguggacgcggcggcgagccgcgggcaggggccggagcccgcgcccggaggcgggguggagggggucggggcucgcggcgucgcacugaaacuuuucguccaacuucugggcuguucucgcuucggaggagccgugguccgcgcgggggaagccgagccgagcggagccgcgagaagugcuagcucgggccgggaggagccgcagccggaggagggggaggaggaagaagagaaggaagaggagagggggccgcaguggcgacucggcgcucggaagccgggcucauggacgggugaggcggcggugugcgcagacagugcuccagccgcgcgcgcuccccaggcccuggcccgggccucgggccggggaggaagaguagcucgccgaggcgccgaggagagcgggccgccccacagcccgagccggagagggagcgcgagccgcgccggccccggucgggccuccgaaaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcggugagccgggcaggaggaaggagccucccucaggguuucgggaaccagaucucucaccaggaaagacugauacagaacgaucgauacagaaaccacgcugccgccaccacaccaucaccaucgacagaacaguccuuaauccagaaaccugaaaugaaggaagaggagacucugcgcagagcacuuuggguccggagggcgagacuccggcggaagcauucccgggcgggugacccagcacggucccucuuggaauuggauucgccauuuuauuuuucuugcugcuaaaucaccgagcccggaagauuagagaguuuuauuucugggauuccuguagacacacccacccacauacauacauuuauauauauauauauuauauauauauaaaaauaaauaucucuauuuuauauauauaaaauauauauauucuuuuuuuaaauuaacagugcuaauguuauuggugucuucacuggauguauuugacugcuguggacuugaguugggaggggaauguucccacucagauccugacagggaagaggaggagaugagagacucuggcaugaucuuuuuuuugucccacuugguggggccaggguccucuccccugcccaggaaugugcaaggccagggcaugggggcaaauaugacccaguuuugggaacaccgacaaacccagcccuggcgcugagccucucuaccccaggucagacggacagaaagacagaucacagguacagggaugaggacaccggcucugaccaggaguuuggggagcuucaggacauugcugugcuuuggggauucccuccacaugcugcacgcgcaucucgcccccaggggcacugccuggaagauucaggagccugggcggccuucgcuuacucucaccugcuucugaguugcccaggagaccacuggcagaugucccggcgaagagaagagacacauuguuggaagaagcagcccaugacagcuccccuuccugggacucgcccucauccucuuccugcuccccuuccuggggugcagccuaaaaggaccuauguccucacaccauugaaaccacuaguucuguccccccaggagaccugguugugugugugugagugguugaccuuccuccauccccugguccuucccuucccuucccgaggcacagagagacagggcaggauccacgugcccauuguggaggcagagaaaagagaaaguguuuuauauacgguacuuauuuaauaucccuuuuuaauuagaaauuaaaacaguuaauuuaauuaaagaguaggguuuuuuuucaguauucuugguuaauauuuaauuucaacuauuuaugagauguaucuuuugcucucucuugcucucuuauuuguaccgguuuuuguauauaaaauucauguuuccaaucucucucucccugaucggugacagucacuagcuuaucuugaacagauauuuaauuuugcuaacacucagcucugcccuccccgauccccuggcuccccagcacacauuccuuugaaauaagguuucaauauacaucuacauacuauauauauauuuggcaacuuguauuuguguguauauauauauauauauguuuauguauauaugugauucugauaaaauagacauugcuauucuguuuuuuauauguaaaaacaaaacaagaaaaaauagagaauucuacauacuaaaucucucuccuuuuuuaauuuuaauauuuguuaucauuuauuuauuggugcuacuguuuauccguaauaauuguggggaaaagauauuaacaucacgucuuugucucuagugcaguuuuucgagauauuccguaguacauauuuauuuuuaaacaacgacaaagaaauacagauauaucuuaaaaaaaaaaaagcauuuuguauuaaagaauuuaauucugaucucaaaaaaaaaaaaaaaaaa

SEQ ID NO: 40 - protein, 188 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDSPRPLCPRCTQHHQRPDPRTCRCRCRRRSFLRCQGRGLELNPDTCRCRKLRR

SEQ ID NO: 41 - RNA, 1704 nt, Homo sapiens
gccguccccgccgccgcugcccgccgccaccggccgcccgcccgcccggcuccuccggccgccuccgcugcgcugcgcugcgcugccugcacccagggcucgggagggggccgcggaggagucgccccccgcgcccggcccccgcccgccgcgcccgggcccgcgccauggggcucuggcugucgccgccccccgcgccgccgggcuagggcgaugcgggcgcccccggcgggcggccccggcgggcaccaugagcccucugcuccgccgccugcugcucgccgcacuccugcagcuggcccccgcccaggccccugucucccagccugaugccccuggccaccagaggaaaguggugucauggauagauguguauacucgcgcuaccugccagccccgggaggugguggugcccuugacuguggagcucaugggcaccguggccaaacagcuggugcccagcugcgugacugugcagcgcugugguggcugcugcccugacgauggccuggagugugugcccacugggcagcaccaaguccggaugcagauccucaugauccgguacccgagcagucagcugggggagaugucccuggaagaacacagccagugugaaugcagaccuaaaaaaaaggacagugcugugaagccagacagccccaggccccucugcccacgcugcacccagcaccaccagcgcccugacccccggaccugccgcugccgcugccgacgccgcagcuuccuccguugccaagggcggggcuuagagcucaacccagacaccugcaggugccggaagcugcgaaggugacacauggcuuuucagacucagcagggugacuugccucagaggcuauaucccagugggggaacaaagaggagccugguaaaaaacagccaagcccccaagaccucagcccaggcagaagcugcucuaggaccugggccucucagagggcucuucugccaucccuugucucccugaggccaucaucaaacaggacagaguuggaagaggagacugggaggcagcaagaggggucacauaccagcucaggggagaauggaguacugucucaguuucuaaccacucugugcaaguaagcaucuuacaacuggcucuuccuccccucacuaagaagacccaaaccucugcauaaugggauuugggcuuugguacaagaacugugacccccaacccugauaaaagagauggaaggagcugucccugccugugucacuguuugucacuguccaggcuggcugguuugggcaugaaugucugcaucacuaaauccagagcuugucuugcucccucauugugcagauggaggaaaugaggacuaaggccccacagcagaucccaggcagggccagaauuauguauucaucacuuucaaguuauugccacgcaugggagucagggauagcccagucaauacagacugccugcccuccugcucuucaccaggguucuuuucuagaaggagacagccuucuguggccagagagcuugggguaggacccagaucuacugagugaccuugcuugucacuaccccugccucucugagcagcaguuuccacaugugcacauagagggaacagaagauugcugugguuggcguccucgggccccagagaaguuugagacuaucuuuacguaauagaaaagaacacuuguucuuccugccaggca

SEQ ID NO: 42 - protein, 207 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 43 - RNA, 1805 nt, Homo sapiens
gccguccccgccgccgcugcccgccgccaccggccgcccgcccgcccggcuccuccggccgccuccgcugcgcugcgcugcgcugccugcacccagggcucgggagggggccgcggaggagucgccccccgcgcccggcccccgcccgccgcgcccgggcccgcgccauggggcucuggcugucgccgccccccgcgccgccgggcuagggcgaugcgggcgcccccggcgggcggccccggcgggcaccaugagcccucugcuccgccgccugcugcucgccgcacuccugcagcuggcccccgcccaggccccugucucccagccugaugccccuggccaccagaggaaaguggugucauggauagauguguauacucgcgcuaccugccagccccgggaggugguggugcccuugacuguggagcucaugggcaccguggccaaacagcuggugcccagcugcgugacugugcagcgcugugguggcugcugcccugacgauggccuggagugugugcccacugggcagcaccaaguccggaugcagauccucaugauccgguacccgagcagucagcugggggagaugucccuggaagaacacagccagugugaaugcagaccuaaaaaaaaggacagugcugugaagccagacagggcugccacuccccaccaccguccccagccccguucuguuccgggcugggacucugcccccggagcacccuccccagcugacaucacccaucccacuccagccccaggccccucugcccacgcugcacccagcaccaccagcgcccugacccccggaccugccgcugccgcugccgacgccgcagcuuccuccguugccaagggcggggcuuagagcucaacccagacaccugcaggugccggaagcugcgaaggugacacauggcuuuucagacucagcagggugacuugccucagaggcuauaucccagugggggaacaaagaggagccugguaaaaaacagccaagcccccaagaccucagcccaggcagaagcugcucuaggaccugggccucucagagggcucuucugccaucccuugucucccugaggccaucaucaaacaggacagaguuggaagaggagacugggaggcagcaagaggggucacauaccagcucaggggagaauggaguacugucucaguuucuaaccacucugugcaaguaagcaucuuacaacuggcucuuccuccccucacuaagaagacccaaaccucugcauaaugggauuugggcuuugguacaagaacugugacccccaacccugauaaaagagauggaaggagcugucccugccugugucacuguuugucacuguccaggcuggcugguuugggcaugaaugucugcaucacuaaauccagagcuugucuugcucccucauugugcagauggaggaaaugaggacuaaggccccacagcagaucccaggcagggccagaauuauguauucaucacuuucaaguuauugccacgcaugggagucagggauagcccagucaauacagacugccugcccuccugcucuucaccaggguucuuuucuagaaggagacagccuucuguggccagagagcuugggguaggacccagaucuacugagugaccuugcuugucacuaccccugccucucugagcagcaguuuccacaugugcacauagagggaacagaagauugcugugguuggcguccucgggccccagagaaguuugagacuaucuuuacguaauagaaaagaacacuuguucuuccugccaggca

SEQ ID NO: 44 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 45 - RNA, 2259 nt, Homo sapiens
augcccugccccugcgcccgccgccgccgccgccgccgcucagcccggcgcgcucuggaggauccugcgccgcggcgcucccgggccccgccgccgccagccgccccgccgcccuccucccgcccccggcaccgccgccagcgcccccgccgcagcgcccgcggcccggcuccucucacuucggggaaggggagggaggagggggacgagggcucuggcggguuuggaggggcugaacaucgcgggguguucuggugucccccgccccgccucuccaaaaagcuacaccgacgcggaccgcggcggcguccucccucgcccucgcuucaccucgcgggcuccgaaugcggggagcucggauguccgguuuccugugaggcuuuuaccugacacccgccgccuuuccccggcacuggcugggagggcgcccugcaaaguugggaacgcggagccccggacccgcucccgccgccuccggcucgcccagggggggucgccgggaggagcccgggggagagggaccaggaggggcccgcggccucgcaggggcgcccgcgcccccaccccugcccccgccagcggaccggucccccacccccgguccuuccaccaugcacuugcugggcuucuucucuguggcguguucucugcucgccgcugcgcugcucccggguccucgcgaggcgcccgccgccgccgccgccuucgaguccggacucgaccucucggacgcggagcccgacgcgggcgaggccacggcuuaugcaagcaaagaucuggaggagcaguuacggucuguguccaguguagaugaacucaugacuguacucuacccagaauauuggaaaauguacaagugucagcuaaggaaaggaggcuggcaacauaacagagaacaggccaaccucaacucaaggacagaagagacuauaaaauuugcugcagcacauuauaauacagagaucuugaaaaguauugauaaugaguggagaaagacucaaugcaugccacgggagguguguauagauguggggaaggaguuuggagucgcgacaaacaccuucuuuaaaccuccauguguguccgucuacagauguggggguugcugcaauagugaggggcugcagugcaugaacaccagcacgagcuaccucagcaagacguuauuugaaauuacagugccucucucucaaggccccaaaccaguaacaaucaguuuugccaaucacacuuccugccgaugcaugucuaaacuggauguuuacagacaaguucauuccauuauuagacguucccugccagcaacacuaccacagugucaggcagcgaacaagaccugccccaccaauuacauguggaauaaucacaucugcagaugccuggcucaggaagauuuuauguuuuccucggaugcuggagaugacucaacagauggauuccaugacaucuguggaccaaacaaggagcuggaugaagagaccugucagugugucugcagagcggggcuucggccugccagcuguggaccccacaaagaacuagacagaaacucaugccagugugucuguaaaaacaaacucuuccccagccaauguggggccaaccgagaauuugaugaaaacacaugccaguguguauguaaaagaaccugccccagaaaucaaccccuaaauccuggaaaaugugccugugaauguacagaaaguccacagaaaugcuuguuaaaaggaaagaaguuccaccaccaaacaugcagcuguuacagacggccauguacgaaccgccagaaggcuugugagccaggauuuucauauagugaagaagugugucguugugucccuucauauuggaaaagaccacaaaugagcuaagauuguacuguuuuccaguucaucgauuuucuauuauggaaaacuguguugccacaguagaacugucugugaacagagagacccuuguggguccaugcuaacaaagacaaaagucugucuuuccugaaccauguggauaacuuuacagaaauggacuggagcucaucugcaaaaggccucuuguaaagacugguuuucugccaaugaccaaacagccaagauuuuccucuugugauuucuuuaaaagaaugacuauauaauuuauuuccacuaaaaauauuguuucugcauucauuuuuauagcaacaacaauugguaaaacucacugugaucaauauuuuuauaucaugcaaaauauguuuaaaauaaaaugaaaauuguauuauaa

SEQ ID NO: 46 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 47 - RNA, 2069 nt, Homo sapiens
aagacacaugcuucugcaagcuuccaugaagguugugcaaaaaaguuucaauccagaguuggguuccagcuuucuguagcuguaagcauugguggccacaccaccuccuuacaaagcaacuagaaccugcggcauacauuggagagauuuuuuuaauuuucuggacaugaaguaaauuuagagugcuuucuaauuucagguagaagacauguccaccuucugauuauuuuuggagaacauuuugauuuuuuucaucucucucuccccaccccuaagauugugcaaaaaaagcguaccuugccuaauugaaauaauuucauuggauuuugaucagaacugauuauuugguuuucugugugaaguuuugagguuucaaacuuuccuucuggagaaugccuuuugaaacaauuuucucuagcugccugaugucaacugcuuaguaaucaguggauauugaaauauucaaaauguacagagaguggguaguggugaauguuuucaugauguuguacguccagcuggugcagggcuccaguaaugaacauggaccagugaagcgaucaucucaguccacauuggaacgaucugaacagcagaucagggcugcuucuaguuuggaggaacuacuucgaauuacucacucugaggacuggaagcuguggagaugcaggcugaggcucaaaaguuuuaccaguauggacucucgcucagcaucccaucgguccacuagguuugcggcaacuuucuaugacauugaaacacuaaaaguuauagaugaagaauggcaaagaacucagugcagcccuagagaaacgugcguggagguggccagugagcuggggaagaguaccaacacauucuucaagcccccuugugugaacguguuccgaugugguggcuguugcaaugaagagagccuuaucuguaugaacaccagcaccucguacauuuccaaacagcucuuugagauaucagugccuuugacaucaguaccugaauuagugccuguuaaaguugccaaucauacagguuguaagugcuugccaacagccccccgccauccauacucaauuaucagaagauccauccagaucccugaagaagaucgcuguucccauuccaagaaacucuguccuauugacaugcuaugggauagcaacaaauguaaauguguuuugcaggaggaaaauccacuugcuggaacagaagaccacucucaucuccaggaaccagcucucugugggccacacaugauguuugacgaagaucguugcgagugugucuguaaaacaccaugucccaaagaucuaauccagcaccccaaaaacugcaguugcuuugagugcaaagaaagucuggagaccugcugccagaagcacaagcuauuucacccagacaccugcagcugugaggacagaugccccuuucauaccagaccaugugcaaguggcaaaacagcaugugcaaagcauugccgcuuuccaaaggagaaaagggcugcccaggggccccacagccgaaagaauccuugauucagcguuccaaguuccccaucccugucauuuuuaacagcaugcugcuuugccaaguugcugucacuguuuuuuucccagguguuaaaaaaaaaauccauuuuacacagcaccacagugaauccagaccaaccuuccauucacaccagcuaaggagucccugguucauugauggaugucuucuagcugcagaugccucugcgcaccaaggaauggagaggaggggacccauguaauccuuuuguuuaguuuuguuuuuguuuuuuggugaaugagaaaggugugcuggucauggaauggcaggugucauaugacugauuacucagagcagaugaggaaaacuguagucucugaguccuuugcuaaucgcaacucuugugaauuauucugauucuuuuuuaugcagaauuugauucguaugaucaguacugacuuucugauuacuguccagcuuauagucuuccaguuuaaugaacuaccaucugauguuucauauuuaaguguauuuaaagaaaauaaacaccauuauucaagcca